(19) United States
(12) Patent Application Publication
(10) Pub. No.: US 2024/0270864 A1
(43) Pub. Date: Aug. 15, 2024

(54) COMPOSITIONS AND METHODS FOR INJECTING PARTICLE SUSPENSIONS

(71) Applicant: Elektrofi, Inc., Boston, MA (US)

(72) Inventors: Paul Brown, Boston, MA (US); Daniel Benjamin Dadon, East Boston, MA (US); James W. Ivey, Reading, MA (US); Moin Khwaja, Boston, MA (US); Lisa Liu, Somerville, MA (US); Jeremy Schieferstein, Medford, MA (US); Sadiqua Shadbar, Allston, MA (US)

(21) Appl. No.: 18/633,795

(22) Filed: Apr. 12, 2024

Related U.S. Application Data
(63) Continuation of application No. PCT/US2023/066395, filed on Apr. 28, 2023.
(60) Provisional application No. 63/336,743, filed on Apr. 29, 2022.

(51) Int. Cl. C07K 16/28; A61K 9/00; A61K 39/00; A61K 47/14; A61K 47/18; A61K 47/20; A61K 47/22; A61K 47/26; A61K 47/44; C07K 16/06; C07K 16/32

(52) U.S. Cl. CPC C07K 16/2887; A61K 9/0019; A61K 47/14; A61K 47/183; A61K 47/20; A61K 47/22; A61K 47/26; A61K 47/44; C07K 16/06; C07K 16/32; A61K 2039/505; A61K 2039/54

(57) ABSTRACT

The present disclosure provides high concentration injectable particle suspensions of therapeutic biologics that permit administration without the need for resuspension. In particular, the compositions disclosed herein are highly concentrated low volume injectable particle suspensions that do not require manual premixing or resuspension prior to administration.

### RELATED APPLICATION(S)

This application is a Continuation of International Application No. PCT/US2023/066395, filed on Apr. 28, 2023, which claims the benefit of U.S. Provisional Application No. 63/336,743, filed on Apr. 29, 2022. The entire teachings of the above applications are incorporated herein by reference.

### TECHNICAL FIELD

The present disclosure relates to compositions and methods that are useful for delivery (e.g., intramuscular or subcutaneous delivery) of biopharmaceutical products for therapy. In particular, the compositions and methods disclosed herein are high concentration, low volume and low syringe force injectable particle suspensions of therapeutic biologics that can be administered to patients without the need for resuspension prior to injection.

### BACKGROUND

Biologics, particularly antibodies, have driven a paradigm shift in the course of drug discovery and development over the last few decades, assisting patients for whom few or no treatment options have previously existed. For example, current monoclonal antibody (mAb) therapies often require large doses which are administered by intravenous (IV) infusion at high-volume and low-concentration, which can take hours to deliver, causing patient discomfort and increasing the risk of infection. Subcutaneous (SC) injection provides a more desirable alternative for delivery since it decreases the burden on hospital and clinical facilities, requiring less time and lowers the risk of complications. However, SC injections of mAb particles require low delivery volumes which necessitate high concentrations that are often difficult to obtain. The requirement of high particle concentrations at low delivery volumes can also result in particle sedimentation that may lead to excessively high injection forces and require manual agitation for particle resuspension. Therefore, a highly concentrated low volume injectable particle composition that does not require manual premixing or resuspension prior to administration is needed.

### SUMMARY

Provided herein is a pharmaceutically effective composition comprising:
a plurality of particles suspended in a pharmaceutically acceptable liquid carrier, wherein the particles (e.g., substantially all of the particles) comprise at least one therapeutic biologic or a salt thereof; and
a flocculation agent, wherein the concentration of the flocculation agent in the composition is less than about 50 mg/mL; and
wherein the concentration of the therapeutic biologic or salt thereof in the composition is greater than about 250 mg/mL.

In one aspect, the disclosure provides a method of treating a disease or condition in a subject in need thereof; comprising administering to the subject a pharmaceutically effective amount of a composition comprising:
a plurality of particles suspended in a pharmaceutically acceptable liquid carrier, wherein the particles (e.g., substantially all of the particles) comprise at least one therapeutic biologic or a salt thereof; and
a flocculation agent, wherein the concentration of the flocculation agent in the composition is less than about 50 mg/mL; and
wherein the concentration of the therapeutic biologic or salt thereof in the composition is greater than about 250 mg/mL.

The present disclosure also provides herein a method of administering a pharmaceutically effective composition comprising:
a plurality of particles suspended in a pharmaceutically acceptable liquid carrier, wherein the particles (e.g., substantially all of the particles) comprise at least one therapeutic biologic or a salt thereof; and
a flocculation agent, wherein the concentration of the flocculation agent in the composition is less than about 50 mg/mL; and
wherein the concentration of the therapeutic biologic or salt thereof in the composition is greater than about 250 mg/mL.

In another aspect, the disclosure provides a pharmaceutically effective composition comprising particles suspended in a pharmaceutically acceptable liquid carrier, wherein the particles comprise:
a first plurality of particles comprising a first therapeutic biologic or a salt thereof;
a second plurality of particles comprising a second therapeutic biologic or a salt thereof; and
a flocculation agent, wherein the concentration of the flocculation agent in the composition is less than about 50 mg/mL;
wherein the concentration of the first and second therapeutic biologics or salts thereof in the composition is greater than about 250 mg/mL.

The present disclosure also provides a kit comprising: a syringe or portable drug delivery injection device and a composition comprising:
a plurality of particles suspended in a pharmaceutically acceptable liquid carrier, wherein the particles (e.g., substantially all of the particles) comprise at least one therapeutic biologic or a salt thereof; and
a flocculation agent, wherein the concentration of the flocculation agent in the composition is less than about 50 mg/mL;
wherein the concentration of the therapeutic biologic or salt thereof in the composition is greater than about 250 mg/mL.

The present compositions, in some embodiments, are useful for administering suspensions of high concentration therapeutic biologic particles by syringe injection, portable drug delivery injection devices, or orally dosed liquid injector capsules, without the need for manual premixing or resuspension prior to administration.

### BRIEF DESCRIPTION OF THE DRAWINGS

FIG. 1 shows an image of particles used in compositions disclosed herein at 1000× magnification.

FIG. 2 shows an image of flocculation volumes in the presence and absence of different flocculation agents (Lecithin, PS80) at multiple concentrations.

FIG. 3 shows a graph indicating the stability of IgG particles alone and in suspension during storage at 40° C. over 12 months.

FIG. 4 shows a plot of parameters for the flocculation agent and protein concentration.

FIG. 5 shows a plot of injectability of the suspension using caprylic triglyceride as the liquid carrier.

FIG. 6 shows plots of flocculation volumes over a period of time at various temperatures.

FIG. 7 shows a plot of injectability of the suspension over a period of time at various temperatures averaging a force of about 2.4 N.

FIG. 8 show plots of injection forces through a variety of needles sizes.

FIG. 9 shows a plot of the injection force in relation to injection time using a 27-gauge UTW needle.

FIGS. 10A and 10B show representative images of the flocculation volume during capsule storage over a period of time.

FIG. 11 shows a graph of the pharmacokinetic profiles for the mAb microparticle suspension (SC injection) and the aqueous mAb SC injection in cohorts of rats (Sprague Dawley).

FIG. 12 shows an image of rituximab particles used in compositions disclosed herein at 30 μm magnification.

FIG. 13 shows an image of rituximab particles sectioned to reveal the internal cross-section.

FIG. 14 shows a plot of the glide force of a 620 mg/mL rituximab suspension with flocculation agent when injected.

FIG. 15 shows an image of rituximab particles used in compositions disclosed herein at 30 μm magnification.

FIG. 16 shows an image of rituximab particles sectioned to reveal the internal cross-section.

FIG. 17 shows a plot of the glide force of a 507 mg/mL rituximab suspension with flocculation agent when injected.

### DETAILED DESCRIPTION

Therapeutic biologics, particularly monoclonal antibody (mAb) therapeutics have dramatically improved the treatment of human disease. However, the delivery of these biologics has been taxing on patients. The standard of administration is often by intravenous (IV) infusion at low concentrations, which can take hours to deliver, causing patient discomfort, and increasing the risk of infection for the patient. Although subcutaneous (SC) delivery by simple injection is preferred, constraints on SC delivery volume (1.5-2.0 mL) necessitate antibody concentrations greater than 100 mg/mL, which are often unfeasible. Solution concentrations exceeding 100 mg/mL are highly viscous, which lead to exceedingly high injection forces and often propagates decomposition of the therapeutic antibody compositions. The utilization of particle suspension technology can deliver therapeutic biologic (e.g., antibody) concentrations >500 mg/mL while preserving full structure and bioactivity of the therapeutic biologic (e.g., mAb), thus, by transforming the delivery of therapeutic biologics from IV to SC can offer advantages to patients, healthcare providers, payers, and pharmaceutical developers. However, therapeutic biologic particles will eventually settle or sediment out of the suspension medium (e.g., pharmaceutically acceptable liquid carrier) over time leading to high injection forces; thus, manual agitation or premixing of the container closure by external means (e.g., vortex or sonic agitation) is required for particle resuspension prior to administration of the composition.

The present disclosure generally relates to pharmaceutically effective compositions and methods comprising: a plurality of particles suspended in a pharmaceutically acceptable liquid carrier, wherein the particles (e.g., substantially all of the particles) comprise at least one therapeutic biologic or a salt thereof; and a flocculation agent, wherein the concentration of the flocculation agent in the composition is less than about 50 mg/mL. In some embodiments, the concentration of the therapeutic biologic or salt thereof in the composition is greater than about 250 mg/mL. In some embodiments, the compositions and methods described herein, further comprising administering a pharmaceutically effective amount of at least one hyaluronan degrading agent (e.g., hyaluronidase) that can be administered simultaneously, sequentially or intermittently with the composition. In some embodiments, the plurality of particles and flocculation agent remain substantially suspended in the non-aqueous liquid carrier for at least one month. In certain embodiments, the composition is administered by syringe injection, portable drug delivery injection device, or orally dosed liquid injector capsules, without the need for manual premixing or resuspension prior to administration. In some embodiments, the injection force of the composition remains substantially the same for at least one month under container closure storage conditions at less than about 40° C. See Examples 2-3 herein, for description of an example composition of the disclosure comprising a plurality of particles suspended with a flocculation agent in a pharmaceutically acceptable liquid carrier.

It will be readily understood that the aspects and embodiments, as generally described herein, are exemplary. The following more detailed description of various aspects and embodiments are not intended to limit the scope of the present disclosure, but is merely representative of various aspects and embodiments. Moreover, the compositions and methods disclosed herein may be changed by those skilled in the art without departing from the scope of the present disclosure. Unless defined otherwise, all technical and scientific terms used herein have the same meaning as is commonly understood by one of skill in the art to which this disclosure belongs. All publications and patents referred to herein are incorporated by reference.

### Definitions

For purposes of the present disclosure, the following definitions will be used unless expressly stated otherwise:

The terms “a”, “an”, “the” and similar referents used in the context of describing the present disclosure are to be construed to cover both the singular and the plural, unless otherwise indicated herein or clearly contradicted by context. All compositions described herein, can be performed in any suitable manner unless otherwise indicated herein or otherwise clearly contradicted by context. The use of any and all examples, or exemplary language (e.g., “such as”) provided herein, is intended merely to better illuminate the present disclosure and does not pose a limitation on the scope of the disclosure otherwise claimed. No language in the present specification should be construed as indicating any unclaimed element is essential to the practice of the disclosure.

The term “about” in relation to a given numerical value, such as for temperature and period of time, is meant to include numerical values within 10% of the specified value.

As used herein, the term “administering” or “administered” means the actual physical introduction of a composition into or onto (as appropriate) a subject. Any and all methods of introducing the composition into subject are contemplated according to the disclosure; the composition is not dependent on any particular means of introduction and is not to be so construed. Means of introduction are known to those skilled in the art and are also exemplified herein.

As used herein, an “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, iso-butyl, sec-butyl, tert-butyl, n-pentyl, tert-pentyl, neo-pentyl, iso-pentyl, sec-pentyl, 3-pentyl, sec-iso-pentyl, active-pentyl, hexyl, heptyl, octyl, ethylhexyl, and the like. A C₁₋₈ straight chained or branched alkyl group is also referred to as a “lower alkyl” group. An alkyl group with two open valences is sometimes referred to as an alkylene group, such as methylene, ethylene, propylene and the like. Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, an alkyl, a halogen, a hydroxyl, a carbonyl (such as a carboxyl, and alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamide, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN and the like. In some embodiments, the term “alkyl” can mean “cycloalkyl” which refers to a non-aromatic carbocyclic ring having 3 to 10 carbon ring atoms, which are carbon atoms bound together to form the ring. The ring may be saturated or have one or more carbon-carbon double bonds. Examples of cycloalkyl include, but not limited to cyclopropyl, cyclobutyl, cyclopentyl, cyclopentenyl, cyclohexyl, cyclohexenyl, and cycloheptyl, as well as bridged and caged saturated ring groups such as norbornyl and adamantyl. As described herein, organic solvents include, but are not limited to aliphatic hydrocarbon solvents, aromatic hydrocarbon solvents, alcohols or alkylalcohols, alkylethers, sulfoxides, alkylketones, alkylacetates, trialkylamines, alkylformates, trialkylamines, or a combination thereof. Aliphatic hydrocarbon solvents can be pentane, hexane, heptane, octane, cyclohexane, and the like or a combination thereof. Aromatic hydrocarbon solvents can be benzene, toluene, and the like or a combination thereof. Alcohols or alkylalcohols include, for example, methanol, ethanol, propanol, butanol, pentanol, hexanol, heptanol, octanol, decanol, amylalcohol, or a combination thereof. Alkylethers include methyl, ethyl, propyl, butyl, and the like, e.g., diethylether, diisopropylether or a combination thereof. Sulfoxides include dimethyl sulfoxide (DMSO), decylmethyl sulfoxide, tetradecylmethyl sulfoxide, and the like or a combination thereof. The term “alkylketone” refers to a ketone substituted with an alkyl group, e.g., acetone, ethylmethylketone, and the like or a combination thereof. The term “alkylacetate” refers to an acetate substituted with an alkyl group, e.g., ethylacetate, propylacetate (n-propylacetate, iso-propylacetate), butylacetate (n-butylacetate, iso-butylacetate, sec-butylacetate, tert-butylacetate), amylacetate (n-pentylacetate, tert-pentylacetate, neo-pentylacetate, iso-pentylacetate, sec-pentylacetate, 3-pentylacetate, sec-iso-pentylacetate, active-pentylacetate), 2-ethylhexylacetate, and the like or a combination thereof. The term “alkylformate” refers to a formate substituted with an alkyl group, e.g., methylformate, ethylformate, propylformate, butylformate, and the like or a combination thereof. The term “trialkylamine” refers to an amino group substituted with three alkyl groups, e.g., triethylamine.

As used herein, an “amino acid” or “residue” refers to any naturally or non-naturally occurring amino acid, any amino acid derivative or any amino acid mimic known in the art. Included are the L- as well as the D-forms of the respective amino acids, although the L-forms are usually preferred. In some embodiments, the term relates to any one of the 20 naturally occurring amino acids: glycine (Gly), alanine (Ala), valine (Val), leucine (Leu), isoleucine (Ile), proline (Pro), cysteine (Cys), methionine (Met), serine (Ser), threonine (Thr), glutamine (Gin), asparagine (Asn), glutamic acid (Glu), aspartic acid (Asp), lysine (Lys), histidine (His), arginine (Arg), phenylalanine (Phe), tryptophan (Trp), and tyrosine (Tyr) in their L-form. In some embodiments, the amino acid sidechain may be a side-chain of Gly, Ala, Val, Leu, Ile, Met, Cys, Ser, Thr, Trp, Phe, Lys, Arg, His, Tyr, Asn, Gln, Asp, Glu, or Pro. In certain embodiments, the amino acid derivative is an amino acid salt, e.g., amino acid salts of hydrochloric acid, phosphoric acid, DL-lactic/glycolic acids, succinic acid, citric acid, sulfuric acid, sodium hydroxide, potassium hydroxide, sodium succinate, sodium phosphate, sodium acetate, sodium citrate, sodium sulfate, or the like.

As used herein, except where the context requires otherwise, the term “comprise” and variations of the term, such as “comprising”, “comprises” and “comprised”, are not intended to exclude further additives, components, integers or steps. The terms “including” and “comprising” may be used interchangeably. As used herein, the phrases “selected from the group consisting of”, “chosen from”, and the like, include mixtures of the specified materials. Where a numerical limit or range is stated herein, the endpoints are included. Also, all values and subranges within a numerical limit or range are specifically included as if explicitly written herein. References to an element in the singular is not intended to mean “one and only one” unless specifically stated, but rather “one or more”. Unless specifically stated otherwise, terms such as “some” refer to one or more, and singular terms such as “a”, “an” and “the” refer to one or more.

The term “oligopeptide” is used to refer to a peptide with fewer members of amino acids as opposed to a polypeptide or protein. Oligopeptides described herein, are typically comprised of about two to about forty amino acid residues. Oligopeptides include dipeptides (two amino acids), tripeptides (three amino acids), tetrapeptides (four amino acids), pentapeptides (five amino acids), hexapeptides (six amino acids), heptapeptides (seven amino acids), octapeptides (eight amino acids), nonapeptides (nine amino acids), decapeptides (ten amino acids), undecapeptides (eleven amino acids), dodecapeptides (twelve amino acids), icosapeptides (twenty amino acids), tricontapeptides (thirty amino acids), tetracontapeptides (forty amino acids), and the like. Oligopeptides may also be classified according to molecular structure: aeruginosins, cyanopeptolins, microcystins, microviridins, microginins, anabaenopeptins and cyclamides, and the like. Homo-oligopeptides are oligopeptides comprising the same amino acid. In particular embodiments, homo-oligopeptides comprise 10 amino acid poly-valine, poly-alanine, and poly-glycine hexamers.

The meaning of the term “peptides” is defined as small proteins of two or more amino acids linked by the carboxyl group of one to the amino group of another. Accordingly, at its basic level, peptide synthesis of whatever type comprises the repeated steps of adding amino acid or peptide molecules to one another or to an existing peptide chain. The term “peptide” generally has from about 2 to about 100 amino acids, whereas a polypeptide or protein has about 100 or more amino acids, up to a full-length sequence which may be translated from a gene. Additionally, as used herein, a peptide can be a subsequence or a portion of a polypeptide or protein. In certain embodiments, the peptide consists of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues. In some embodiments, the peptide is from about 30 to about 100 amino acids in length.

As used herein, the term “pharmaceutically acceptable” refers to compositions that are within the scope of sound medical judgment, suitable for use in contact with the tissues of human beings and animals without excessive toxicity, irritation, allergic response, or other problem or complication, commensurate with a reasonable benefit/risk ratio. Preferably, the term “pharmaceutically acceptable” means approved by a regulatory agency of a federal or state government or listed in the U.S. Pharmacopeia or other generally recognized pharmacopeia for use in animals, and more particularly in humans.

The meaning of the term “protein” is defined as a linear polymer built from about 20 different amino acids. The type and the sequence of amino acids in a protein are specified by the DNA that produces them. In certain embodiments, the sequences can be natural and unnatural. The sequence of amino acids determines the overall structure and function of a protein. In some embodiments, proteins can contain 50 or more residues. In particular embodiments, proteins can contain greater than about 101 residues in length. A protein's net charge can be determined by two factors: 1) the total count of acidic amino acids vs. basic amino acids; and 2) the specific solvent pH surroundings, which expose positive or negative residues. As used herein, “net positively or net negatively charged proteins” are proteins that, under non-denaturing pH surroundings, have a net positive or net negative electric charge. In general, those skilled in the art will recognize that all proteins may be considered “net negatively charged proteins”, regardless of their amino acid composition, depending on their pH and/or solvent surroundings. For example, different solvents can expose negative or positive side chains depending on the solvent pH. Proteins are preferably selected from any type of enzyme or antibodies or fragments thereof showing substantially the same activity as the corresponding enzyme or antibody. Proteins may serve as a structural material (e.g., keratin), as enzymes, as hormones, as transporters (e.g., hemoglobin), as antibodies, or as regulators of gene expression. Proteins are required for the structure, function, and regulation of cells, tissues, and organs. In some embodiments, the protein is a therapeutic biologic. In certain embodiments, the protein is bovine serum albumin (BSA) or human serum albumin (HSA).

The term “substantially” as used herein, refers to a majority of, or mostly, as in at least about 50%, 60%, 70%, 80%, 90%, 95%, 96%, 97%, 98%, 99%, 99.5%, 99.9%, 99.99%, or at least about 99.999% or more.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. A phrase such as “embodiment” does not imply that such embodiment applies to all configurations of the subject technology. A disclosure relating to an embodiment may apply to all embodiments, or one or more embodiments. A phrase such as an embodiment may refer to one or more embodiments and vice versa.

### Particles

Unless otherwise defined, all terms of art, notations and other scientific terminology used herein, are intended to have the meanings commonly understood by those of skill in the art to which this disclosure pertains. In some cases, terms with commonly understood meanings are defined herein, for clarity and/or for ready reference, and the inclusion of such definitions herein, should not necessarily be construed to represent a substantial difference over what is generally understood in the art. The techniques and procedures described or referenced herein, are generally well understood and commonly employed using conventional methodology by those skilled in the art. As appropriate, procedures involving the use of commercially available kits and reagents are generally carried out in accordance with manufacturer defined protocols and/or parameters unless otherwise noted. As used herein, the phrase “and/or” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The terms “particle” or “particles” or “microparticle” or “microparticles” as used herein, interchangeably in the broadest sense, refer to a discrete body or bodies. The particles described herein are circular and of controlled dispersity with a characteristic size from sub-micrometers to tens of micrometers, in contrast to, e.g., a porous monolithic “cake”, which is typically produced during conventional lyophilization. This morphology allows for a flowable powder (e.g., characterized by low Hausner ratios) without post-processing. In some embodiments, the term “particle” refers to a quantity of a protein or proteins, e.g., therapeutic biologic or therapeutic biologics which is either in a state of matter that is substantially solid as compared to a liquid droplet.

As disclosed herein, a therapeutic biologic, also known as a biologic medical product, or biopharmaceutical, is any pharmaceutical drug product manufactured in, extracted from or semi synthesized from biological sources. Therapeutic biologics can include a wide range of products such as vaccines, blood and blood components, allergenics, somatic cells, gene therapy, tissues, and recombinant therapeutic proteins. Biologics can be isolated from a variety of natural sources, e.g., a human, animal, or microorganism, and may be produced by biotechnology methods or other technologies. In some embodiments of the disclosure, the therapeutic biologic is an antibody or fragment thereof. In some embodiments, the antibody is a mammalian or human antibody, e.g., bovine IgG, human IgG, or a monoclonal antibody (mAb). In certain embodiments, the antibody is Rituximab or Trastuzumab. In particular embodiments, the therapeutic biologic is a fragment of an antibody. In some embodiments, the compositions described herein, comprise at least two pluralities of particles (e.g., first and second pluralities of particles) suspended in a pharmaceutically acceptable liquid carrier, wherein each plurality of particles comprise at least one therapeutic biologic (e.g., at least one different therapeutic biologic relative to particles in the other pluralities). In some embodiments, the compositions described herein, comprise at least two pluralities of particles (e.g., first and second pluralities of particles) suspended in a pharmaceutically acceptable liquid carrier, wherein each plurality of particles comprise at least one antibody, e.g., a plurality of rituximab particles and a plurality of trastuzumab particles.

The terms “antibody” and “immunoglobulin” are used interchangeably in the broadest sense and include monoclonal antibodies, polyclonal antibodies, multivalent antibodies, and multispecific antibodies, regardless of how they are produced, i.e., using immunization, recombinant, synthetic methodologies. Antibodies can be gamma globulin proteins that are found in blood, or other bodily fluids of vertebrates that function in the immune system to bind antigen, hence identifying and/or neutralizing foreign objects. Antibodies can be assigned to different classes or isotypes. There are five classes of immunoglobulins: IgA, IgD, IgE, IgG, and IgM, having heavy chains designated alpha, delta, epsilon, gamma, and mu, respectively. The gamma class is further divided into subclasses based on the differences in sequences and function, e.g., humans express the following subclasses: IgG1, IgG2, IgG3, IgG4, IgA1, and IgA2. In some embodiments of the disclosure, the IgG antibody is an IgG1 antibody. In certain embodiments of the disclosure, the IgG antibody is a monoclonal IgG antibody. The L chain from any vertebrate species can be assigned to one of two clearly distinct types, e.g., kappa and lambda, based on the amino acid sequences of their constant domains.

The recognized immunoglobulin genes include the kappa, lambda, alpha, gamma, delta, epsilon and mu constant region genes, as well as the myriad immunoglobulin variable region genes. In some embodiments, light chains are classified as either kappa or lambda. In some embodiments, heavy chains are classified as gamma, mu, alpha, delta, or epsilon, which in turn define the immunoglobulin classes, IgG, IgM, IgA, IgD and IgE, respectively. In particular embodiments of the disclosure, the antibody is an IgG antibody.

An exemplary antibody (immunoglobulin) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light” chain, domain, region and component are used interchangeably, are abbreviated by “VL” or “V_{L}” and refer to the light chain of an antibody or antibody fragment. Similarly, terms “variable heavy” chain, domain, region and component are used interchangeably, are abbreviated by “VH” or “V_{H}” and refer to the heavy chain of an antibody or antibody fragment. Antibodies are generally a heterotetrameric glycoprotein composed of two identical light (L) chains and two identical heavy (H) chains. Each L chain is linked to an H chain by one covalent disulfide bond. The two H chains are linked to each other by one or more disulfide bonds depending on the H chain isotype. Each H and L chain also has regularly spaced intra-chain disulfide bridges. H and L chains define specific Ig domains. In particular, each H chain has at the N-terminus, a variable domain (VH) followed by three constant domains (CH) for each of the alpha and gamma chains and four CH domains for p and c isotypes. Each L chain has at the N-terminus, a variable domain (VL) followed by a constant domain (CL) at its other end. The V_{L} is aligned with the VH and the CL is aligned with the first constant domain of the heavy chain (CHL). The constant domain includes the Fc portion which comprises the carboxy-terminal portions of both H chains held together by disulfides. The effector functions of antibodies such as ADCC are determined by sequences in the Fc region, which is also the part recognized by Fc receptors (FcR) found on certain types of cells.

As disclosed herein, the pairing of a VH and VL together form a “variable region” or “variable domain” including the amino-terminal domains of the heavy or light chain of the antibody. The variable domain of the heavy chain may be referred to as “VH”. The variable domain of the light chain may be referred to as “VL”. The V domain contains an “antigen binding site” which affects antigen binding and defines specificity of a particular antibody for its particular antigen. V regions span about 110 amino acid residues and consist of relatively invariant stretches called framework regions (FRs) (generally about 4) of 15-30 amino acids separated by shorter regions of extreme variability called “hypervariable regions” (generally about 3) that are each generally 9-12 amino acids long. The FRs largely adopt a p-sheet configuration and the hypervariable regions form loops connecting, and in some cases forming part of, the p-sheet structure. In certain embodiments, the “hypervariable region” refers to the regions of an antibody variable domain which are hypervariable in sequence and/or form structurally defined loops. Generally, antibodies comprise six hypervariable regions; three in the VH (H1, H2, H3), and three in the VL (L1, L2, L3). “Framework” or “FR” residues are those variable domain residues other than the hypervariable region residues defined herein.

The terms “full length antibody”, “intact antibody” and “whole antibody” are used herein, interchangeably, to refer to an antibody in its substantially intact form, not as antibody fragments as defined above. The terms particularly refer to an antibody with heavy chains that contain the Fc region. A full length antibody can be a native sequence antibody or an antibody variant. In certain embodiments, an “intact” or “whole” antibody is one which comprises an antigen-binding site as well as a CL and at least heavy chain constant domains, CH1, CH2 and CH3. The constant domains may be native sequence constant domains, e.g., human native sequence constant domains, or amino acid sequence variants thereof.

As indicated above, the term antibody as used herein, unless otherwise stated or clearly contradicted by context, includes fragments of an antibody that retain the ability to specifically interact, such as, bind to the antigen. It has been shown that the antigen-binding function of an antibody may be performed by fragments of a full-length antibody. Examples of binding fragments encompassed within the term “antibody” include (i) a Fab′ or Fab fragment, a monovalent fragment consisting of the light chain variable domain (VL), heavy chain variable domain (VH), light chain constant region (CL) and heavy chain constant region domain 1 (CH1) domains, or a monovalent antibody as described in WO 2007/059782; (ii) F(ab′)₂ fragments, bivalent fragments comprising two Fab fragments linked by a disulfide bridge at the hinge region; (iii) an Fd fragment consisting essentially of the VH and CH1 domains; (iv) an Fv fragment consisting essentially of the V_{L} and VH domains of a single arm of an antibody, (v) a dAb fragment Ward et al., Nature 341, 544-546 (1989), which consists essentially of a VH domain and is also called domain antibody Holt et al; Trends Biotechnol. 2003 November; 21(11):484-90; (vi) camelid or nanobodies Revets et al; Expert Opin Biol Ther. 2005 January; 5(1):111-24 and (vii) an isolated complementarity determining region (CDR). Furthermore, although the two domains of the Fv fragment, V_{L} and VH, are coded for by separate genes, they may be joined, using recombinant methods, by a synthetic linker that enables them to be made as a single protein chain in which the V_{L} and VH regions pair to form monovalent molecules (known as single chain antibodies or single chain Fv (scFv), see for instance Revets et al; Expert Opin Biol Ther. 2005 January; 5(1):111-24 and Bird et al., Science 242, 423-426 (1988). Such single chain antibodies are encompassed within the term antibody unless otherwise noted or clearly indicated by context. Although such fragments are generally included within the meaning of antibody, they are collectively and each independently are unique features of the present invention, exhibiting different biological properties and utility. These and other useful antibody fragments in the context of the present invention are discussed further herein.

As disclosed herein, “whole antibody fragments including a variable domain” include Fab, Fab′, F(ab′)₂, and Fv fragments; diabodies; linear antibodies, single-chain antibody molecules; and multispecific antibodies formed from antibody fragments. The “Fab fragment” consists of an entire L chain along with the variable region domain of the H chain (VH), and the first constant domain of one heavy chain (CHI). Each Fab fragment is monovalent with respect to antigen binding, i.e., it has a single antigen-binding site. A “Fab′ fragment” differs from Fab fragments by having additional few residues at the carboxy terminus of the CHI domain including one or more cysteines from the antibody hinge region. Fab′-SH is the designation herein for Fab′ in which the cysteine residue(s) of the constant domains bear a free thiol group. A “F(ab′)₂ fragment” roughly corresponds to two disulfide linked Fab fragments having divalent antigen-binding activity and is still capable of cross-linking antigen. An “Fv” is the minimum antibody fragment which contains a complete antigen-recognition and binding site. This fragment consists of a dimer of one heavy and one light chain variable region domain in tight, non-covalent association. In a single-chain Fv (scFv) species, one heavy and one light chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody. “Single-chain Fv” also abbreviated as “sFv” or “scFv” are antibody fragments that comprise the VH and VL antibody domains connected to form a single polypeptide chain. In particular embodiments, the scFv polypeptide further comprises a polypeptide linker between the VH and VL domains which enables the scFv to form the desired structure for antigen binding. In some embodiments, a “single variable domain” is half of an Fv (comprising only three CDRs specific for an antigen) that has the ability to recognize and bind antigen, although at a lower affinity than the entire binding site.

In some embodiments, “diabodies” refer to antibody fragments with two antigen-binding sites, which fragments comprise a heavy-chain variable domain (VH) connected to a light-chain variable domain (V_{L}) in the same polypeptide chain (VH-V_{L}). The small antibody fragments are prepared by constructing sFv fragments with short linkers (about 5-10 residues) between the VH and VL domains such that inter-chain but not intra-chain pairing of the V domains is achieved, resulting in a bivalent fragment, i.e., fragment having two antigen-binding sites. In some embodiments, diabodies may be bivalent or bispecific. In certain embodiments, bispecific diabodies are heterodimers of two “crossover” sFv fragments in which the VH and V_{L} domains of the two antibodies are present on different polypeptide chains. Triabodies and tetrabodies are also generally known in the art.

“Antigen binding fragments” of antibodies as described herein, comprise only a portion of an intact antibody, generally including an antigen binding site of the intact antibody and thus retaining the ability to bind antigen. Exemplary examples of antibody fragments encompassed by the present definition include but are not limited to: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment which consists of a VH domain; (vii) isolated CDR regions; (viii) F(ab′)₂ fragments, a bivalent fragment including two Fab′ fragments linked by a disulfide bridge at the hinge region; (ix) single chain antibody molecules, e.g. single chain Fv; scFv; (x) “diabodies” with two antigen binding sites, comprising a heavy chain variable domain (VH) connected to a light chain variable domain (VL) in the same polypeptide chain; (xi) “linear antibodies” comprising a pair of tandem Fd, segments (VH—CH1-VH—CH1) which, together with complementary light chain polypeptides, form a pair of antigen binding regions. In some embodiments, an “antigen binding site” generally refers to a molecule that includes at least the hypervariable and framework regions that are required for imparting antigen binding function to a V domain. An antigen binding site may be in the form of an antibody or an antibody fragment, (such as a dAb, Fab, Fd, Fv, F(ab′)₂ or scFv) in a method described herein. In some embodiments, an antigen-binding fragment competes with intact antibody, e.g., with the intact antibody from which the fragment was derived, for antigen binding.

The term “fragment” refers to a part or portion of an antibody or antibody chain comprising fewer amino acid residues than an intact or complete antibody or antibody chain. As used herein, the term “fragment” of an antibody molecule includes Fc fragments and antigen-binding fragments of antibodies, for example, an antibody light chain variable domain (VL), an antibody heavy chain variable domain (VH), a single chain antibody (scFv), a F(ab′)2 fragment, a Fab fragment, an Fd fragment, an Fv fragment, a single domain antibody fragment (DAb), a one-armed (monovalent) antibody, or any antigen-binding molecule formed by combination, assembly or conjugation of such antigen binding fragments.

In some embodiments, the term “single-chain Fv” or “scFv” or “single chain” antibody can refer to antibody fragments comprising the VH and VL domains of antibody, wherein these domains are present in a single polypeptide chain. Generally, the Fv polypeptide further comprises a polypeptide linker between the VH and VL domains which enables the sFv to form the desired structure for antigen binding. For a review of sFv, see Pluckthun, THE PHARMACOLOGY OF MONOCLONAL ANTIBODIES, vol. 113, Rosenburg and Moore eds. Springer-Verlag, New York, pp. 269-315 (1994).

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies (mAbs) are highly specific, being directed against a single antigenic site or determinant on the antigen. In addition to their specificity, the monoclonal antibodies are advantageous in that they may be synthesized uncontaminated by other antibodies. Monoclonal antibodies may be prepared by the hybridoma methodology. The monoclonal antibodies may also be isolated from phage antibody libraries using molecular engineering techniques. The monoclonal antibodies of the disclosure may be generated by recombinant DNA methods and are sometimes referred to as “recombinant antibodies” or “recombinant monoclonal antibodies” as described herein. In some embodiments, a monoclonal antibody is a single species of antibody wherein every antibody molecule recognizes the same epitope because all antibody producing cells are derived from a single B-lymphocyte cell line. The methods for generating monoclonal antibodies (mAbs) generally begin along the same lines as those for preparing polyclonal antibodies. In some embodiments, rodents such as mice and rats are used in generating monoclonal antibodies. In certain embodiments, rabbit, sheep, or frog cells are used in generating monoclonal antibodies. The use of rats is well known and may provide certain advantages. Mice, e.g., BALB/c mice, are routinely used and generally give a high percentage of stable fusions. In some embodiments of the disclosure, the antibody is a monoclonal antibody. In particular embodiments of the disclosure, the IgG antibody is monoclonal.

In some embodiments, recombinant antibody fragments may be isolated from phage antibody libraries using techniques well known in the art. See, for example, Clackson et al., 1991, Nature 352: 624-628; Marks et al., 1991, J. Mol. Biol. 222: 581-597. Recombinant antibody fragments may be derived from large phage antibody libraries generated by recombination in bacteria (Sblattero and Bradbury, 2000, Nature Biotechnology 18:75-80; and as described herein). Polynucleotides encoding the VH and VL components of antibody fragments, i.e., scFv, may be used to generate recombinant full length immunoglobulins using methods known in the art (see, for example, Persic et al., 1997, Gene 187: 9-18).

An “isolated antibody” is one that has been identified and separated and/or recovered from a component of its pre-existing environment. Contaminant components are materials that would interfere with therapeutic uses for the antibody, and may include enzymes, hormones, and other proteinaceous or nonproteinaceous solutes.

As used herein, a “human antibody” refers to an antibody that possesses an amino acid sequence that corresponds to that of an antibody produced by a human. Human antibodies can be produced using various techniques known in the art, including phage-display libraries. Human antibodies can be prepared by administering the antigen to a transgenic animal that has been modified to produce such antibodies in response to antigenic challenge, but whose endogenous loci has been disabled. “Humanized” forms of non-human, e.g., rodent, antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some embodiments, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

An “affinity matured” antibody is one with one or more alterations in one or more hypervariable region thereof that result in an improvement in the affinity of the antibody for antigen, compared to a parent antibody that does not possess those alterations. In some embodiments, affinity matured antibodies can have micromolar affinities for the target antigen. In some embodiments, affinity matured antibodies can have nanomolar or even picomolar affinities for the target antigen. Affinity matured antibodies are produced by procedures known in the art.

A “blocking” antibody or an “antagonist” antibody is one that inhibits or reduces biological activity of the antigen it binds. In some embodiments, blocking antibodies or antagonist antibodies substantially or completely inhibits the biological activity of the antigen. An “agonist antibody”, as used herein, is an antibody, which mimics at least one of the functional activities of a polypeptide of interest. As used herein, the term “inhibition”, “inhibit”, “inhibiting” and the like in reference to a protein-inhibitor, e.g., antagonist, interaction means negatively affecting, e.g., decreasing, the activity or function of the protein relative to the activity or function of the protein in the absence of the inhibitor. In some embodiments, inhibition refers to reduction of a disease or the symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

“Binding affinity” generally refers to the strength of the sum total of noncovalent interactions between a single binding site of a molecule, e.g., an antibody, and its binding partner, e.g., an antigen. Unless indicated otherwise, as used herein, “binding affinity” refers to intrinsic binding affinity, which reflects a 1:1 interaction between members of a binding pair, e.g., antibody and antigen. The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. “Epitope” generally refers to that part of an antigen that is bound by the antigen binding site of an antibody. In some embodiments, an epitope may be “linear” in the sense that the hypervariable loops of the antibody CDRs that form the antigen binding site bind to a sequence of amino acids as in a primary protein structure. In some embodiments, the epitope is a “conformational epitope”, i.e., one in which the hypervariable loops of the CDRs bind to residues as they are presented in the tertiary or quaternary protein structure.

In some embodiments of the foregoing compositions, the protein, e.g., therapeutic biologic is an antibody. In some embodiments, the antibody includes but are not limited to 3F8, Abagovomab, Abatacept, Abciximab, Abituzumab, Abrezekimab, Abrilumab, Acritumomab, Actoxumab, Abituzumab, Adalimumab-adbm, Adalimumab-atto, Adalimumab-bwwb, Adecatumumab, Ado-trastuzumab emtansine, Aducanumab, Afasevikumab, Afelimomab, Aflibercept, Afutuzumab, Alacizumab pegol, ALD518, Alefacept, Alemtuzumab, Alirocumab, Altumomab pentetate, Amatuximab, Anatumomab mafenatox, Andecaliximab, Anetumab ravtansine, Anifrolumab, Anrukinzumab, Ansuvimab, Apolizumab, Aprutumab ixadotin, Arcitumomab, Ascrinvacumab, Aselizumab, Atezolizumab, Atidortoxumab, Atinumab, Atlizumab, Atoltivimab, Atorolimumab, Avelumab, Azintuxizumab vedotin, Bapineuzumab, Basiliximab, Bavituximab, BCD-100, Bectumomab, Begelomab, Belantamab mafodotin, Belatacept, Belimumab, Bemarituzumab, Benralizumab, Bermekimab, Bersanlimab, Bertilimumab, Besilesomab, Bevacizumab, Bevacizumab-awwb, Bezlotoxumab, Biciromab, Bimagrumab, Bimekizumab, Birtamimab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Capromab pendetide, Carlumab, Carotuximab, Catumaxomab, cBR96-doxorubicin immunoconjugate, Cedelizumab, Cemiplimab, Cergutuzumab amunaleukin, Cergutuzumab amunaleukin, Certolizumab pegol, Cetrelimab, Cetuximab, Cibisatamab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, CR6261, Crizanlizumab, Crotedumab, Cusatuzumab, Dacetuzumab, Daclizumab, Dalotuzumab, Dapirolizumab pegol, Daratumumab, Dectrekumab, Demcizumab, Denileukin diftitox, Denintuzumab mafodotin, Denosumab, Depatuxizumab mafodotin, Derlotuximab biotin, Detumomab, Dezamizumab, Dinutuximab, Diridavumab, Domagrozumab, Dorlimomab aritox, Dostarlimab, Drozitumab, DS-8201, Duligotumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epoetin-alfa, Epoetin-alfa-epbx, Epratuzumab, Eptinezumab, Erenumab, Erlizumab, Ertumaxomab, Etanercept, Etanercept-szzs, Etaracizumab, Etigilimab, Etrolizumab, Evinacumab, Evolocumab, Exbivirumab, Factor VIII Fc fusion protein, Factor IX Fc fusion protein, Fanolesomab, Faralimomab, Faricimab, Farletuzumab, Fasinumab, Felvizumab, Fezakinumab, Fibatuzumab, Ficlatuzumab, Figitumumab, Filgrastim, Filgrastim-sndz, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ibalizumab, IBI308, Ibritumomab tiuxetan, Icrucumab, Idarucizumab, Ifabotuzumab, Igovomab, Iladatuzumab vedotin, IMAB362, Imalumab, Imaprelimab, Imciromab, Imgatuzumab, Inclacumab, Indatuximab ravtansine, Indusatumab vedotin, Inebilizumab, Infliximab, Infliximab-abda, Infliximab-dyyb, Infliximab-qbtx, Intetumumab, Inolimomab, Inotuzumab ozogamicin, Ipilimumab, Iomab-B, Iratumumab, Isatuximab, Iscalimab, Istiratumab, Itolizumab, Ixekizumab, Keliximab, Labetuzumab, Lacnotuzumab, Ladiratuzumab vedotin, Lambrolizumab, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab amadotin, Lutikizumab, Maftivimab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odesivimab, Odesivimab-ebgn, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pegfilgrastim-jmdb, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, Relatlimab, Remtolumab, Reslizumab, Rilonacept, Rilotumumab, Rinucumab, Risankizumab, Rituximab, Rituximab-abbs, Rituximab-arrx, Rituximab-pvvr, Rivabazumab pegol, Rivabazumab pegol, Robatumumab, Rmab, Roledumab, Romilkimab, Romiplostim, Romosozumab, Rontalizumab, Rosmantuzumab, Rovalpituzumab tesirine, Rovalpituzumab tesirine, Rovelizumab, Rozanolixizumab, Ruplizumab, Sacituzumab govitecan, Samalizumab, Samrotamab vedotin, Sapelizumab, Sarilumab, Satralizumab (SA237), Satumomab pendetide, Secukinumab, Selicrelumab, Seribantumab, Setoxaximab, Setrusumab, Sevirumab, Sibrotuzumab, SGN-CD19A, SGN-CD33A, SHP647, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Tafasitamab, Talacotuzumab, Talizumab, Tamtuvetmab, Tanezumab, Taplitumomab paptox, Tarextumab, Tavolimab, Tefibazumab, Telimomab aritox, Telisotuzumab vedotin, Tenatumomab, Teneliximab, Teplizumab, Tepoditamab, Teprotumumab, Tesidolumab, Tetulomab, Tezepelumab, TGN1412, Tibulizumab, Ticilimumab, Tildrakizumab, Tigatuzumab, Timigutuzumab, Timolumab, Tiragotumab, Tislelizumab, Tisotumab vedotin, TNX-650, Tocilizumab, Tomuzotuximab, Toralizumab, Tosatoxumab, Tositumomab, Tovetumab, Tralokinumab, Trastuzumab, Trastuzumab-anns, Trastuzumab-dkst, Trastuzumab-dttb, Trastuzumab emtansine, Trastuzumab-pkrb, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Xentuzumab, XMAB-5574, Zalutumumab, Zanolimumab, Zatuximab, Zenocutuzumab, Ziralimumab, Zolbetuximab (IMAB362, Claudiximab), Ziv-aflibercept, or Zolimomab aritox.

In some embodiments of the foregoing compositions, the antibody is monoclonal. In certain embodiments, the monoclonal antibody includes but are not limited to 3F8, 8119, Abatacept, Abagovomab, Abciximab, Abituzumab, Adalimumab-adbm, Adalimumab-atto, Adalimumab-bwwb, Abrilumab, Actoxumab, Abituzumab, Abrezekimab, Abrilumab, Actoxumab, Adalimumab, Adecatumumab, Ado-trastuzumab emtansine, Aducanumab, Afasevikumab, Afelimomab, Aflibercept, Afutuzumab, Alacizumab pegol, ALD518, Alefacept, Alemtuzumab, Alirocumab, Altumomab pentetate, Amatuximab, Anatumomab mafenatox, Andecaliximab, Anetumab ravtansine, Anifrolumab, Anrukinzumab (IMA-638), Ansuvimab, Apolizumab, Arcitumomab, Ascrinvacumab, Aselizumab, Atezolizumab, Atidortoxumab, Atinumab, Atlizumab (tocilizumab), Atoltivimab, Atorolimumab, Avelumab, Bapineuzumab, Basiliximab, Bevacizumab, Bevacizumab-awwb, BCD-100, Bectumomab, Begelomab, Belatacept, Belimumab, Bemarituzumab, Benralizumab, Bermekimab, Bersanlimab, Bertilimumab, Besilesomab, Bezlotoxumab, Biciromab, Bimagrumab, Bimekizumab, Birtamimab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camrelizumab, Canakinumab, Cantuzumab mertansine, Cantuzumab ravtansine, Caplacizumab, Capromab pendetide, Carlumab, Carotuximab, Catumaxomab, Cedelizumab, Cemiplimab, Certolizumab pegol, Cetrelimab, Cetuximab, Cibisatamab, Cirmtuzumab, Ch.14.18, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, CR6261, Crizanlizumab, Crotedumab, Cusatuzumab, Dacetuzumab, Daclizumab, Dalotuzumab, Dapirolizumab pegol, Daratumumab, Dectrekumab, Demcizumab, Denileukin diftitox, Denintuzumab mafodotin, Denosumab, Derlotuximab biotin, Detumomab, Dezamizumab, Dinutuximab, Diridavumab, Domagrozumab, Dorlimomab aritox, Dostarlimab, Drozitumab, Duligotumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epoetin-alfa, Epoetin-alfa-epbx, Epratuzumab, Eptinezumab, Erenumab, Erlizumab, Ertumaxomab, Etanercept, Etanercept-szzs, Etaracizumab, Etigilimab, Etrolizumab, Evinacumab, Evolocumab, Exbivirumab, Factor VIII Fc fusion protein, Factor IX Fc fusion protein, Fanolesomab, Faralimomab, Faricimab, Farletuzumab, Fasinumab, FBTA05, Felvizumab, Fezakinumab, Fibatuzumab, Ficlatuzumab, Figitumumab, Filgrastim, Filgrastim-sndz, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ibalizumab, IBI308, Ibritumomab tiuxetan, Icrucumab, Idarucizumab, Ifabotuzumab, Igovomab, IMAB362, Imalumab, Imaprelimab, Imciromab, Imgatuzumab, Inclacumab, Indatuximab ravtansine, Indusatumab vedotin, Inebilizumab, Infliximab, Infliximab-abda, Infliximab-dyyb, Infliximab-qbtx, Intetumumab, Inolimomab, Inotuzumab ozogamicin, Ipilimumab, Iratumumab, Isatuximab, Iscalimab, Istiratumab, Itolizumab, Ixekizumab, Keliximab, Labetuzumab, Lacnotuzumab, Lambrolizumab, Lampalizumab, Lanadelumab, Landogrozumab, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lutikizumab, Maftivimab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odesivimab, Odesivimab-ebgn, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pegfilgrastim-jmdb, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Tetulomab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, Relatlimab, Remtolumab, Reslizumab, Rilonacept, Rilotumumab, Rinucumab, Risankizumab-rzaa, Rituximab, Rituximab-abbs, Rituximab-arrx, Rituximab-pvvr, Robatumumab, Rmab, Roledumab, Romilkimab, Romiplostim, Romosozumab, Rontalizumab, Rosmantuzumab, Rovelizumab, Rozanolixizumab, Ruplizumab, Sacituzumab govitecan, Samalizumab, Sarilumab, Satralizumab (SA237), Satumomab pendetide, Secukinumab, Selicrelumab, Seribantumab, Setoxaximab, Setrusumab, Sevirumab, Sibrotuzumab, SGN-CD19A, SGN-CD33A, SHP647, Sifalimumab, Siltuximab, Simtuzumab, Siplizumab, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Tafasitamab, Talacotuzumab, Talizumab, Tamtuvetmab, Tanezumab, Taplitumomab paptox, Tarextumab, Tavolimab, Tefibazumab, Telimomab aritox, Tenatumomab, Teneliximab, Teplizumab, Tepoditamab, Teprotumumab, Tesidolumab, Tetulomab (lilotomab), Tezepelumab, TGN1412, Tibulizumab, Ticilimumab (tremelimumab), Tildrakizumab, Tigatuzumab, Timigutuzumab, Timolumab, Tiragotumab, Tislelizumab, TNX-650, Tocilizumab (atlizumab), Tomuzotuximab, Toralizumab, Tosatoxumab, Tositumomab, Tovetumab, Tralokinumab, Trastuzumab, Trastuzumab-anns, Trastuzumab-dkst, Trastuzumab-dttb, Trastuzumab emtansine, Trastuzumab-pkrb, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Xentuzumab, XMAB-5574, Zalutumumab, Zanolimumab, Zatuximab, Zenocutuzumab, Ziralimumab, Zolbetuximab (IMAB362, Claudiximab), Ziv-aflibercept, Zolimomab aritox or the corresponding anti-drug antibody in a sample from a human patient. In certain embodiments, the monoclonal antibody is Rituximab, Rituximab-abbs, Rituximab-arrx, or Rituximab-pvvr. In some embodiments, the monoclonal antibody is Atoltivimab, Maftivimab, Odesivimab-ebgn, or a combination thereof.

In some embodiments, the monoclonal antibody is a biosimilar. In some embodiments, the biosimilar includes but are not limited to Adalimumab-adbm, Adalimumab-atto, Adalimumab-bwwb, Bevacizumab-awwb, Epoetin alfa-epbx, Etanercept-szzs, Infliximab-abda, Infliximab-dyyb, Infliximab-qbtx, Filgrastim-sndz, Odesivimab-ebgn, Pegfilgrastim-jmdb, Pegfilgrastim-bmez, Risankizumab-rzaa, Rituximab-abbs, Rituximab-arrx, Rituximab-pvvr, Trastuzumab-anns, Trastuzumab-dttb, Trastuzumab-pkrb, or Trastuzumab-dkst. In certain embodiments, the active biosimilar substance is Adalimumab, Bevacizumab, Enoxaparin sodium, Epoetin alfa, Epoetin zeta, Etanercept, Filgrastim, Follitropin alfa, Infliximab, Insulin glargine, Insulin lispro, Pegfilgrastim, Risankizumab, Rituximab, Rituximab-abbs, Rituximab-arrx, Rituximab-pvvr, Somatropin, Teriparatide, Trastuzumab, Trastuzumab-anns, Trastuzumab-dttb, Trastuzumab-pkrb, or Trastuzumab-dkst. In some embodiments, the biosimilar is Rituximab, Rituximab-abbs, Rituximab-arrx, or Rituximab-pvvr. In certain embodiments, the biosimilar is Trastuzumab-anns, Trastuzumab-dttb, Trastuzumab-pkrb, or Trastuzumab-dkst.

In some embodiments, the targeting moiety is an antibody from an intact polyclonal antibody, an intact monoclonal antibody, an antibody fragment, a single chain Fv (scFv) mutant, a multispecific antibody, a bispecific antibody, a chimeric antibody, a humanized antibody, a human antibody, a fusion protein comprising an antigenic determinant portion of an antibody, or other modified immunoglobulin molecules comprising antigen recognition sites.

In certain embodiments, the therapeutic biologic is ledipasvir/sofosbuvir, insulin glargine, lenalidomide, pneumococcal 13-valent conjugate vaccine, fluticasone/salmeterol, elvitegravir/cobicistat/emtricitabine/tenofovir alafenamide, emtricitabine, rilpivirine and tenofovir alafenamide, emtricitabine/tenofovir alafenamide, grazoprevir/elbasvir, coagulation factor VIIa recombinant, epoetin alfa, Aflibercept or etanercept.

In some embodiments, the therapeutic biologic is Abatacept, AbobotulinumtoxinA, Agalsidase beta, Albiglutide, Aldesleukin, Alglucosidase alfa, Alteplase (cathflo activase), Anakinra, Asfotase alfa, Asparaginase, Asparaginase Erwinia chrysanthemi, Becaplermin, Belatacept, Collagenase, Collagenase clostridium histolyticum, Darbepoetin alfa, Denileukin diftitox, Dornase alfa, Dulaglutide, Ecallantide, Efgartigimod alfa-fcab, Elosulfase alfa, Etanercept-szzs, Filgrastim, Filgrastim-sndz, Galsulfase, Glucarpidase, Idursulfase, IncobotulinumtoxinA, Interferon alfa-2b, Interferon alfa-n3, Interferon beta-1a, Interferon beta-1b, Interferon gamma-1b, Laronidase, Methoxy polyethylene glycol-epoetin beta, Metreleptin, Ocriplasmin, OnabotulinumtoxinA, Oprelvekin, Palifermin, Parathyroid hormone, Pegaspargase, Pegfilgrastim, Peginterferon alfa-2a, Peginterferon alfa-2a co-packaged with ribavirin, Peginterferon alfa-2b, Peginterferon beta-1a, Pegloticase, Rasburicase, Reteplase, Rilonacept, RimabotulinumtoxinB, Romiplostim, Sargramostim, Sebelipase alfa, Tbo-filgrastim, Tenecteplase, or Ziv-aflibercept.

The protein, e.g., therapeutic biologic in the particles as disclosed herein, has an activity per unit of about 0.5 to about 1.0, about 0.75 to about 1.0 activity per unit, or about 0.9 to about 1.0 activity per unit. Activity is measured relative to the same protein prior to particle formation. In some embodiments, the protein has an activity per unit of about 0.5 to about 1.0. The term “activity” refers to the ratio of a functional or structural aspect of a protein, e.g., an antibody, antibody fragment, bovine serum albumin (BSA), or human serum albumin (HSA), at two points in time. The denominator of the ratio corresponds to a measure of the functional or structural aspect of the protein in the aqueous feed solution, immediately in advance of droplet formation. The numerator of the ratio corresponds to the same measure of a functional or structural aspect of the protein at a later point in time, e.g., immediately after particle formation.

In some embodiments, substantially all of the particles have less than about 10%, e.g., less than about 9%, 8%, 7%, 6%, 5%, 4%, 3%, 2%, or 1% aggregation of the protein, e.g., therapeutic biologic after processing. In some embodiments, substantially all of the particles have less than about 5% aggregation of the therapeutic biologic. In certain embodiments, substantially all of the particles have less than about 3% aggregation of the therapeutic biologic. In some embodiments, substantially all of the particles have less than about 1% aggregation of the therapeutic biologic. In certain embodiments, substantially all of the particles are substantially free from any aggregation of the therapeutic biologic. In some embodiments, substantially all of the particles have less than about 0.5% aggregation of the therapeutic biologic.

In some embodiments, substantially all of the particles have less than about 10% fragmentation of the protein, e.g., therapeutic biologic after processing. In some embodiments, substantially all of the particles have less than about 3% fragmentation of the therapeutic biologic. In certain embodiments, substantially all of the particles have less than about 1% fragmentation of the therapeutic biologic. In some embodiments, substantially all of the particles are substantially free from any fragmentation of the therapeutic biologic. In some embodiments, substantially all of the particles have less than about 5% fragmentation of the therapeutic biologic. Suitable methods for measuring aggregation and fragmentation of a therapeutic biologic therapeutic biologic can be accomplished by using size-exclusion chromatography (SEC).

In certain embodiments, the composition provides less than about 5% change in charge variants in the population of a protein, e.g., therapeutic biologic (e.g., less than about 4, 3, 2, or 1%) as compared to the therapeutic biologic prior to particle formation. In some embodiments, the particles have less than about 5% change in charge variants in the population of a therapeutic biologic after processing. Charge variants may be acidic, basic, or neutral, and the variation may be caused by post-translation modifications at terminal amino acids, such as asparagine deamidation or lysine glycation. For example, charge variants include the loss of a positive charge by the loss of a C-terminal lysine residue, covalent bonding of the amine portions of two lysine residues by reducing sugars, or the conversion of an N-terminal amine to a neutral amide by the cyclization of N-terminal glutamines. Negative charges on proteins, e.g., antibodies or fragments, can appear by the conversion of asparagine residues to aspartic acid and/or isoaspartic residues via a deamidation reaction.

In some embodiments, substantially all of the particles have less than about 5% change in charge variants of the protein, e.g., therapeutic biologic, compared to an aqueous composition comprising at least one therapeutic biologic in soluble (monomeric) form prior to particle formation. In some embodiments, substantially all of the particles have less than about 3% change in charge variants of the therapeutic biologic. In certain embodiments, substantially all of the particles have less than about 1% change in charge variants of the therapeutic biologic. In some embodiments, substantially all of the particles are substantially free from any change in charge variants of the therapeutic biologic. Exemplary methods of measuring charge variants include cation exchange chromatography (CIEX), where the variants are quantified by dividing the area under the peak corresponding to the variant, e.g., acidic, basic, or neutral population by the cumulative area contained beneath all peaks in the sample spectrum. Changes in charge variant population percentage between two samples, e.g., Sample A and Sample B, are computed as the numerical difference in the respective population variant percentages, i.e., by subtracting the specific variant, e.g., acidic, percentage of Sample B from the specific variant, e.g., acidic, percentage of Sample A, or vice versa. In certain embodiments, the analysis may be extended similarly for all variants within a population.

The particles according to the disclosure are circular. Circularity can serve as an indicator of the shape of the particle. The particles described herein, can have a characteristic circularity, e.g., have a relative shape, that is substantially circular. This characteristic describes and defines the form of a particle on the basis of its circularity. The circularity is 1.0 when the particle has a completely circular structure. Particles according to the disclosure, have a circularity of about 0.80 to about 1.00, 0.90 to about 1.00, 0.95 to about 1.00, 0.96 to about 1.00, 0.97 to about 1.00, 0.98 to about 1.00, or 0.99 to about 1.00. In some embodiments, the circularity of substantially all of the particles is about 0.85 to about 1.00. In some embodiments, the circularity of substantially all of the particles about 0.90 to about 1.00. In certain embodiments, the circularity of substantially all of the particles is about 0.95 to about 1.00. In particular embodiments, the circularity of substantially all of the particles is about 0.98 to about 1.00. In certain embodiments, the circularity of substantially all of the particles is about 1.00. The diameter and the circularity of the particles can be determined by the processing of an image observed under an electron microscope or the like or a flow-type particle image analyzer. The circularity can also be determined by subjecting particles to circularity measurement and averaging the resulting values. For example, circularity (circ) can be calculated using the following formula:

$$c i r c = 4 * π * \frac{Area}{P e r {imeter}^{2}} . \qquad Eq . 1$$

The term “perimeter”, as used herein, refers to the boundary of a closed plane figure or the sum of all borders of a two-dimensional image. As used herein, the term “area”, refers to the cross sectional area of a two-dimensional image of a particle. The circularity of a particle can also be described as the ratio of the smallest dimension of the particle to its largest diameter. For a perfect circle, the ratio is 1. The percentage circularity can be calculated by multiplying the circularity by 100. The circularity can be calculated, for example, by measuring the aspect ratio using any software adapted to deal with images, for example, images obtained by microscopy, in particular, scanning electron microscopy (SEM) or transmission electron microscopy (TEM). In some embodiments, methods of measuring particle circularity include image analysis of scanning electron micrographs of the particles in which the average roundness is calculated on the basis of the cross-sectional shapes of the particles projected onto the plane of the image. Such roundness factors can be extended to identify the corresponding circularity.

In particular embodiments of the disclosure, the particles as described herein, have a surface morphology that is smooth rather than ridged or wrinkled. A person of ordinary skill in the field of this disclosure can readily assess the surface morphology of the disclosed particles using routine and standard techniques.

In certain embodiments, the particles have a diameter of about 0.1 to about 1000 μm, e.g., about 0.1 to about 900, 800, 700, 600, 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, or about 0.2 μm. In some embodiments, the particles have a diameter of about 1 to about 100 μm, e.g., about 2, 3, 4, 5, 6, 7, 8, 9, 10, 11, 12, 13, 14, 15, 16, 17, 18, 19, 20, 25, 30, 35, 40, 45, or 50 to about 100 μm. In some embodiments, the particles have a diameter of about 4 to about 100 μm. In certain embodiments, the particles have a diameter of about 10 to about 100 μm. In certain embodiments, the particles have a diameter of about 20 to about 50 μm. Methods of measuring the particle size and distribution include imaging flow cytometry, laser diffraction, and image analysis of scanning electron micrographs of the particles in which an average spherical radius or diameter can be calculated on the basis of the cross-sectional areas of the particles projected onto the plane of the image.

As used herein, the term “dispersity index” (DI) is a parameter characterizing the degree of non-uniformity of a size distribution of particles. The polydispersity index (PDI), “population dispersity” or “span”, e.g., D10, D50, D90, can also mean a value that indicates the breadth of the particle size distribution. Particle size distribution are reported by D10, D50, D90, and the mean particle size in μm, with the values representing the percentage of particles that are smaller than the indicated D-number, e.g. the D10 particle size is the particle diameter at which 10% of the mass is composed of particles with a diameter less than this value, the D50 particle size is the particle diameter at which 50% of the mass is composed of particles with a diameter less than this value and the D90 particle size is the particle diameter at which 90% of the mass is composed of particles with a diameter less than this value. The D10, D50, and D90 particle size distribution can be measured using a laser light scattering particle sizer.

Particle diameter and dispersity can also impact injectability, e.g., syringeability. Particles are often recommended to be at least 3-10 times smaller than the inner diameter of the needle. Even if a small fraction of the particle population is larger than the inner diameter of the needle, they may clog the needle and cause the entire dosage to go to waste. In certain embodiments of the disclosure described herein, high concentrations of the therapeutic biologic in the compositions are achieved by mixing particles of various sizes.

As used herein, the terms “moisture” and “water” may be used interchangeably. In some embodiments, the moisture content of substantially all of the particles is less than about 7% by weight, e.g., less than about 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% by weight. In some embodiments, substantially all of the particles have less than about 7% moisture by weight. In certain embodiments, substantially all of the particles have less than about 5% moisture by weight. In certain embodiments, substantially all of the particles have less than about 3% moisture by weight. In some embodiments, substantially all of the particles have less than about 1% moisture by weight. Exemplary methods for the measurement of moisture content include chemical titration methods, e.g., Karl Fischer titration involving an oven. A variety of solvents, including water, may also be measured using weight loss methods involving thermal excitation. Exemplary methods include Thermogravimetric Analysis with Infrared Spectroscopy (TGA-IR) or Gas Chromatography Flame Ionization Detector/Mass Spectrometry (GC-FID/MS).

The term “internal void space” or “internal void” as used herein, which in contrast to a pore or porosity, does not communicate with the surface of a solid (e.g., particle), and will not contribute to porosity or surface area. In some embodiments, substantially all of the particles have less than about 10% internal void spaces, e.g., less than about 9, 8, 7, 6, 5, 4, 3, 2, or 1% internal void spaces. In some embodiments, substantially all of the particle is substantially free from any internal void spaces. Suitable methods for determining internal void space can be accomplished by using Focused Ion Beam Scanning Electron Microscopy (FIB-SEM), which can be used to visualize “accessible” pores and “inaccessible” void spaces. Another method for determining internal void space can be accomplished by Gas displacement pycnometry which is a common analytical technique that uses a gas displacement method to measure volume. Inert gases, such as helium or nitrogen, are used as the displacement medium. True volume is total volume minus volume accessible to the gas. Density is calculated by dividing sample weight with the true volume. The sample is sealed in the instrument compartment of a known volume, the appropriate inert gas is admitted, and then expanded into another precision internal volume. The pressure before and after expansion is measured and used to compute the sample volume. Dividing this volume into the sample weight gives the gas displacement density. Cross-sections of typical particles of the disclosure indicate an absence of any internal void spaces as shown by FIB-SEM or gas pycnometry using helium at temperatures at about 22° C. to provide densities typically averaging about 1.3 g/cm³ with standard deviations at about 0.0005 g/cm³. For example, internal void space can be calculated using the following formula: internal void space=Aᵥ/Aₚ, where A, is the total area of void spaces and Aₚ is the total area of the particle. Cross-sections of typical particles of the disclosure can be obtained and characterized in a number of ways, for example, in International Application Nos. PCT/US2020/15957 (Pub. No. WO 2020/160323) and PCT/US2021/027755 (WO 2021/212019), the contents of each of which are hereby incorporated by reference in their entireties.

In some embodiments, substantially all of the particles have less than about 10% internal void spaces. In certain embodiments, substantially all of the particles have less than about 5% internal void spaces. In certain embodiments, substantially all of the particles have less than about 3% internal void spaces. In some embodiments, substantially all of the particles have less than about 1% internal void spaces. In certain embodiments, substantially all of the particles are substantially free from any internal void spaces.

In some embodiments, substantially all of the particles have greater than about 50% protein, e.g., therapeutic biologic by weight, e.g., greater than about 55, 60, 65, 70, 75, 80, 85, 86, 87, 88, 89, 90, 91, 92, 93, 94, 95, 96, 97, 98, or 99% therapeutic biologic by weight. In some embodiments, substantially all of the particles have greater than about 60% therapeutic biologic by weight. In certain embodiments, substantially all of the particles have greater than about 70% therapeutic biologic by weight. In some embodiments, substantially all of the particles have greater than about 80% therapeutic biologic by weight. In particular embodiments, substantially all of the particles have greater than about 90% therapeutic biologic by weight. In certain embodiments, substantially all of the particles have greater than about 95% therapeutic biologic by weight. In certain embodiments, substantially all of the particles have greater than about 98% therapeutic biologic by weight. In some embodiments, substantially all of the particles have greater than about 99% therapeutic biologic by weight.

The term “excipient” refers to an additive to a particle formulation, which may be useful in achieving a desired modification to the characteristics or morphology of the particles. Such modifications include, but are not limited to, physical stability, chemical stability, and therapeutic efficacy. Exemplary excipients include, but are not limited to a carbohydrate, a pH adjusting agent, a salt, a chelator, a surfactant, a protein stabilizer, an emulsifier, an antiseptic, an amino acid, an antioxidant, a paraben, a bactericide, a fungicide, a preservative, or a combination thereof. In some embodiments, substantially all of the particles further comprise one or more excipients. In some embodiments, substantially all of the particles further comprise a carbohydrate, a pH adjusting agent, a salt, a chelator, a surfactant, a protein stabilizer, an emulsifier, an amino acid, an antioxidant, or a combination thereof. In some embodiments, substantially all of the particles further comprise at least a carbohydrate and an amino acid. In some embodiments, substantially all of the particles further comprise at least a surfactant and an amino acid. In certain embodiments, substantially all of the particles further comprise at least one amino acid.

In some embodiments, the carbohydrate may be from the families of monosaccharides, disaccharides, oligosaccharides, or polysaccharides. In some embodiments, the carbohydrate is dextran, trehalose, sucrose, agarose, mannitol, lactose, sorbitol, maltose, starch, alginates, xanthan, galactomanin, agar, agarose, or a combination thereof. In certain embodiments, the carbohydrate is dextran, trehalose, sucrose, agarose, mannitol, lactose, sorbitol, maltose, or a combination thereof. In certain embodiments, the carbohydrate is trehalose, cyclodextrins, hydroxypropyl beta-cyclodextrin, sulfobutylether beta-cyclodextrin, methyl beta-cyclodextrin, or a combination thereof. In particular embodiments, the carbohydrate is trehalose, sucrose, sorbitol, or a combination thereof. Cyclodextrins are available in three different forms α, β, and γ based on the number of number of glucose monomers. The number of glucose monomers in α, β, and γ cyclodextrin can be 6, 7, or 8, respectively.

The terms “pH adjusting agent” or “buffering species” or “buffering agent” are used herein, interchangeably in the broadest sense. In some embodiments, substantially all of the particles comprise a pH adjusting agent. In some embodiments, substantially all of the particles comprise one or more pH adjusting agents. In certain embodiments, the pH adjusting agent is acetate, citrate, glutamate, glycinate, histidine, lactate, maleate, phosphate, succinate, tartrate, bicarbonate, aluminum hydroxide, phosphoric acid, hydrochloric acid, DL-lactic/glycolic acids, phosphorylethanolamine, tromethamine, imidazole, glyclyglycine, monosodium glutamate, sodium hydroxide, potassium hydroxide, or a combination thereof. In certain embodiments, the pH adjusting agent is acetate, citrate, histidine, phosphate, succinate, hydrochloric acid, sodium hydroxide, or a combination thereof. In particular embodiments, the pH adjusting agent is histidine, hydrochloric acid, or a combination thereof. In certain embodiments, the pH adjusting agent is histidine·HCl, succinate, phosphate, or a combination thereof. In some embodiments, the pH levels of the composition as disclosed herein, is about 5.0 to about 8.0. In some embodiments, the pH levels of the composition as disclosed herein, is about 5.5 to about 7.0. In certain embodiments, the pH level is about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5. In certain embodiments, the pH level is about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0. In particular embodiments, the pH level is about 5.0, 5.5, 6.0, 6.5, or 7.0.

In some embodiments, the salt is sodium chloride, calcium chloride, potassium chloride, sodium hydroxide, stannous chloride, magnesium sulfate, sodium glucoheptonate, sodium pertechnetate, guanidine hydrochloride, potassium hydroxide, magnesium chloride, potassium nitrate, or a combination thereof. In particular embodiments, the salt is sodium chloride, sodium hydroxide, potassium nitrate, magnesium chloride, or a combination thereof. In certain embodiments, the salt is sodium chloride.

In some embodiments, the chelator is disodium edetate, ethylenediaminetetraacetic acid, pentetic acid, or a combination thereof.

In some embodiments, the surfactant includes, but is not limited to: (i) cationic surfactants such as; cetyltrimethylammonium chloride, hexadecyltrimethylammonium chloride, benzalkonium chloride, benzethonium chloride, dioctadecyldimethylammonium bromide; (ii) anionic surfactants such as magnesium stearate, sodium stearate, sodium dodecyl sulfate, dioctyl sodium sulfosuccinate, sodium myreth sulfate, perfluorooctanesulfonate, alkyl ether phosphates, Aerosol-OT (sodium bis(2-ethylhexyl) sulfosuccinate); (iii) non-ionic surfactants such as polysorbates, polyethylene glycol tert-octylphenyl ethers, lecithin, alkylphenol ethoxylates, fatty alcohol ethoxylates (octaethylene glycol monododecyl ether, cocamide diethanolamine, poloxamers, glycerolmonostearate, fatty amines, fatty acid esters of sorbitol (sorbitan monolaurate, Tween 80, Tween 20, poloxamers (nonionic triblock copolymers composed of a central hydrophobic chain of polyoxypropylene (poly(propylene oxide)) flanked by two hydrophilic chains of polyoxyethylene (poly(ethylene oxide)) e.g., poloxamer 188, poloxamer 407; and (iv) zwitterionic surfactants such as cocamidopropyl hydroxysultaine, and 3-[(3-Cholamidopropyl)dimethylammonio]-1-propanesulfonate (CHAPS). In some embodiments, the surfactant is polysorbate, magnesium stearate, sodium dodecyl sulfate, alkylphenol ethoxylates, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, phosphatidylcholine, polyglycerol polyricinoleate, siloxanes, cetyl polyethylene glycol/polypropylene glycol-10/1 dimethicone triglyceride, bis-polyethylene glycol/polypropylene glycol-14/14 dimethicone, bis-(glyceryl/lauryl) glyceryl lauryl dimethicone (&) caprylic/capric triglyceride, cetyl polyethylene glycol/polypropylene glycol-10/1 dimethicone, phospholipids, or a combination thereof. In certain embodiments, the surfactant is polysorbate, docusate, poloxamer or lecithin. In some embodiments, the surfactant is polysorbate 20, polysorbate 60, or polysorbate 80. In certain embodiments, the surfactant is polysorbate 20 or polysorbate 80, e.g., Tween 20, Tween 60, Tween 80. In certain embodiments, the fatty acid ester of sorbitol is a sorbitan ester, e.g., span 20, span 40, span 60, or span 80. In some embodiments, the surfactant is an ionic surfactant. In particular embodiments, the surfactant is polysorbate 80, polysorbate 20, or poloxamer 188. In some embodiments, the surfactant is a sorbitan ester, polysorbate, poloxamers, polyethylene glycol tert-octylphenyl ethers, lecithin, sodium stearate, magnesium stearate, or a combination thereof. In some embodiments, the surfactant is polysorbate, sorbitan ester, poloxamer, or a combination thereof.

In some embodiments, the protein stabilizer is acetyltryptophanate, caprylate, N-acetyltryptophan, trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, or a combination thereof. In some embodiments, the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, cyclodextrin, hydroxypropyl beta-cyclodextrin, sulfobutylether beta-cyclodextrin, methyl beta-cyclodextrin, acetyltryptophanate, caprylate, N-acetyltryptophan, propyleneglycol, glucose star polymer, silicone polymer, polydimethylsiloxane, carboxymethylcellulose, poly(glycolic acid), poly(lactic-co-glycolic acid), polylactic acid, polycaprolactone (PCL), polyvinylpyrrolidone (PVP), ficoll, dextran, or a combination thereof. In particular embodiments, the protein stabilizer is trehalose, cyclodextrin, hydroxypropyl beta-cyclodextrin, sulfobutylether beta-cyclodextrin, methyl beta-cyclodextrin, or a combination thereof. In some embodiments, the protein stabilizer is hydroxypropyl beta-cyclodextrin, methyl beta-cyclodextrin, or a combination thereof. The stabilizers, used synonymously with the term “stabilizing agent”, as described herein, can be a salt, a carbohydrate, saccharides or amino acids, preferably a carbohydrate or saccharide admitted by the authorities as a suitable additive or excipient in pharmaceutical compositions. In certain embodiments, the PEG is PEG 200, PEG 300, PEG 3350, PEG 8000, PEG 10000, PEG 20000, or a combination thereof. The term “stabilizer” refers to an excipient or a mixture of excipients which stabilizes the physical and/or chemical properties of a protein, e.g., an antibody or antibody fragment. In some embodiments, stabilizers prevent, e.g., degradation of the protein during mixing, and/or storage of the particulate matter. Exemplary stabilizers include, but are not limited to, sugars, salts, hydrophobic salts, detergents, reducing agents, cyclodextrins, polyols, carboxylic acids, and amino acids. A “stable” formulation or composition as described herein, refers to a particle formulation or composition comprising the particles in which the protein retains an acceptable portion of its essential physical, chemical, or biological properties over an acceptable period of time. In the case of proteins, e.g., exemplary methods of assessing stability are reviewed in (i) Peptide and Protein Drug Delivery, 247-301, Vincent Lee Ed., Marcel Dekker, Inc., New York, NY, 1991, and (ii) Jones, A., Adv. Drug Delivery Rev. 10: 29-90 (1993). In some embodiments, chemical stability of a protein is assessed by measuring the size distribution of the sample at several stages. These include, e.g., before particle formation (assessment of the aqueous feed solution), immediately after particle formation, and again after a period of storage, where storage takes place either within or in the absence of a suspension carrier medium.

Examples of emulsifiers suitable for use in the particles include, but are not limited to, lipophilic agents having an HLB of less than 7, such as mixed fatty acid monoglycerides; mixed fatty acid diglycerides; mixtures of fatty acid mono- and diglycerides; lipophilic polyglycerol esters; glycerol esters including glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan ester including sorbitan monostearate, sorbitan sesquioleate; fatty acids and their soaps including stearic acid, palmitic acid, and oleic acid; and mixtures thereof glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan ester including sorbitan monostearate, sorbitan sesquioleate; fatty acids and their soaps including stearic acid, palmitic acid, and oleic acid; phospholipids; or a combination thereof. In some embodiments, the emulsifier is polysorbate (polysorbate 80, polysorbate 60, polysorbate 20, e.g., Tween 80, Tween 60, Tween 20), sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof. In some embodiments, the fatty acid ester of sorbitol is a sorbitan ester, e.g., span 20, span 40, span 60, or span 80. In certain embodiments, the emulsifier is polysorbate 80, sorbitan monooleate, or a combination thereof.

In some embodiments, the antiseptic is phenol, m-cresol, benzyl alcohol, 2-phenyloxyethanol, chlorobutanol, neomycin, benzethonium chloride, gluteraldehyde, beta-propiolactone, or a combination thereof.

In certain embodiments, the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or various salts thereof (arginine hydrochloride, arginine glutamate, or the like), or a combination thereof. In some embodiments, the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof. In still some embodiments, the amino acid is arginine, histidine, proline, asparagine, or a combination thereof. In particular embodiments, the amino acid is histidine. In some embodiments, the amino acid is alanine, glutamic acid, methionine, threonine, tryptophan, asparagine, arginine, histidine, glycine, glutamine, proline, or a combination thereof. In some embodiments, the amino acid is arginine and histidine. In certain embodiments, the amino acid is histidine.

In some embodiments, the antioxidant is glutathione, ascorbic acid, cysteine, N-acety-L-tryptophanate, tocopherol, histidine, methionine, pentetic acid, or a combination thereof. In some embodiments, the paraben is a parahydroxybenzoate. In certain embodiments, the bactericide is benzalkonium chloride (cationic surfactants), hypochlorites, peroxides, alcohols, phenolic compounds (e.g., carbolic acid), benzyl benzoate, or a combination thereof. In particular embodiments, the bactericide is benzalkonium chloride or benzyl benzoate. In certain embodiments, the fungicide is acibenzolar, 2-phenylphenol, anilazine, carvone, natamycin, potassium azide, or a combination thereof.

In some embodiments, the preservative is sodium nitrate, sulfur dioxide, potassium sorbate, sodium sorbate, sodium benzoate, benzoic acid, methyl hydroxybenzoate, thimerosal, parabens, formaldehyde, castor oil, or a combination thereof. In certain embodiments, the preservative is methyl hydroxybenzoate, thimerosal, a paraben, formaldehyde, castor oil, or a combination thereof.

The plurality of particles comprising at least one protein, e.g., therapeutic biologic described herein, can be prepared and characterized in a number of ways, as well as any methods of forming the particles disclosed in, for example, in International Application Nos. PCT/US2017/063150 (Pub. No. WO 2018/098376), PCT/US2018/043774 (Pub. No. WO 2019/023392), PCT/US2019/033875 (Pub. No. WO 2019/226969), PCT/US2020/15957 (Pub. No. WO 2020/160323), PCT/US2020/050508 (Pub. No. WO 2021/050953), PCT/US2021/16878 (WO 2021/158959), PCT/US2021/018806 (WO 2021/168271), PCT/US2021/027755 (WO 2021/212019), and PCT/US2022/072755 (WO 2022/256840), the contents of each of which are hereby incorporated by reference in their entireties.

While each of the elements of the present disclosure is described herein, as containing multiple embodiments, it should be understood that, unless indicated otherwise, each of the embodiments of a given element of the present disclosure is capable of being used with each of the embodiments of the other elements of the present disclosure and each such use is intended to form a distinct embodiment of the present disclosure.

It will be understood by one of ordinary skill in the relevant arts that other suitable modifications and adaptations to the composition described herein are readily apparent from the description of the disclosure contained herein, in view of information known to the ordinarily skilled artisan and may be made without departing from the scope of the disclosure or any embodiment thereof.

### Pharmaceutical Compositions of the Disclosure

Injectable particle suspensions can exhibit variations in sedimentation, which may impact the use or administration of the drug composition. In particular, high concentration, low volume and low syringe force injectable particle suspensions of therapeutic biologics will settle or sediment out of the suspension medium over some period of time, thus, requiring premixing or resuspension prior to injection. The sedimentation of high particle concentrations at low delivery volumes may also lead to high injection forces and propagate decomposition of the therapeutic biologic in the composition. As used herein, the term “sediment” or “sedimentation” is the process of particles settling or being densely deposited at the bottom of a container closure, e.g., vial, cartridge, syringe, portable drug delivery injection device, or the like. The settling of densely deposited particles generally leads to excessively high injection forces or syringe blockage and requires manual agitation or premixing prior to administration of the composition.

Flocculation or “particle flocculation” is a phenomenon that arises from the interplay between the interfacial chemistry and environmental conditions that govern particle-particle interactions. Flocculation volume, as used herein, is a measure of the amount of particle agglomerates that are occupied by the particulate dispersion, expressed as a percentage of the total fluid volume. More specifically, the flocculation volume (F) is the ratio of the volume of particle agglomerates (Vu) to the total fluid volume (Vo). The flocculation volume can decrease over time based on the sedimentation properties of the particle agglomerates. In a flocculated suspension, the particles undergo collisions leading to particle agglomeration or agglomerates, that increase in size, e.g., increase in flocculation volume. The term “agglomerates” refers to a cluster of particles that are loosely coherent. The loose networks of particle agglomerates maintain enough particle-to-particle distance to prevent particle sedimentation that can lead to high injection forces. When the flocculation volume is equal to 1, the yield stress (the stress at which the flocculation volume will undergo deformation) can be measured to indicate the shear stress that is needed to make the suspension flow. Measurements for yield stress can be acquired by a parallel plate rheometer (ANTON PAAR™ MCR 92). An increase in yield stress indicates an increase in agglomerates. Other approaches for measuring agglomerates include, but are not limited to, laser diffraction, relaxation NMR, microscopy and small-angle scattering.

In the composition disclosed herein, the process of agglomeration can be controlled by the use of flocculation agents. Flocculation agents, or flocculating agents, are chemicals that promote flocculation by causing suspended particles in the liquid to agglomerate, thereby forming agglomerates. In non-aqueous particle suspensions, the breakup and redispersion of agglomerates is governed by the shear stress imparted on the flocculated particle system. Shear stress is caused by a force acting on the material's surface, which causes deformation. In these systems, shear can be attributed to the movement of particles or agglomerates through a velocity gradient in the non-aqueous suspending medium or near any interface (e.g., the container closure, other particles, etc.). Shear stress can be introduced to the flocculation volume, for example, mechanically, and can be controlled experimentally.

The compositions disclosed herein comprise a flocculating agent and are generally high concentration, low volume and low syringe force injectable particle suspensions of therapeutic biologics that permit administration without the need for manual agitation or premixing prior to injection or administration of the composition. The compositions beneficially maintain stable flocculation volumes that avoid particle sedimentation that can result in blockage of syringes, portable drug delivery injection devices, or orally dosed liquid injector capsules.

In certain embodiments, the disclosure relates to a composition, e.g., pharmaceutical composition, comprising a plurality of particles suspended in a pharmaceutically acceptable liquid carrier, wherein the particles (e.g., substantially all of the particles) comprise a therapeutic biologic (e.g., a therapeutic biologic disclosed herein) and a flocculation agent (e.g., a flocculation agent disclosed herein).

The phrase “pharmaceutically acceptable liquid carrier” as used herein, means a pharmaceutically acceptable material, composition or vehicle, such as a liquid, diluent, excipient, or solvent. Each carrier must be “acceptable” in the sense of being compatible with the other ingredients of the compositions and not injurious to the patient. One skilled in the art may dilute or increase the volume of the therapeutic biologic with an inert material in the particles. These diluents could include carbohydrates, especially trehalose, mannitol, a-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

In some embodiments, pharmaceutical compositions for administration include non-aqueous solutions of the active therapeutic biologics in water-soluble form. Preferably, suspensions of the active therapeutic biologics may be prepared as appropriate oily injection compositions. Suitable lipophilic solvents or vehicles include fatty oils (e.g., sesame oil, corn oil), or fatty acid esters (e.g., ethyl oleate or triglycerides), or liposomes. The proper viscosity can be maintained by the maintenance of the required particle size in the case of injection, and by the use of a flocculation agents. The prevention of the action of microorganisms can be brought about by various antibacterial and antifungal agents, for example, parabens, chlorobutanol, phenol, sorbic acid, thimerosal, and the like.

This disclosure includes the use of “pharmaceutically acceptable salts” or “salts” of therapeutic biologics in the composition of the present disclosure. The term “pharmaceutically acceptable salt” or “salts” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, or other acids. In some embodiments, pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of a therapeutic biologic. As another example, the salt may comprise less than one inorganic or organic acid molecule per molecule of base, such as two molecules of a therapeutic biologic per molecule of tartaric acid. In certain embodiments, contemplated salts of the disclosure include, but are not limited to, arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, L-lysine, magnesium, 4-(2-hydroxy ethyl)morpholine, piperazine, potassium, 1-(2-hydroxyethyl)pyrrolidine, sodium, triethanolamine, tromethamine, or zinc salts. In some embodiments, contemplated salts of the disclosure include, but are not limited to, Na, Ca, K, Mg, Zn or other metal salts. In further embodiments, contemplated salts of the disclosure include, but are not limited to, alkyl, dialkyl, trialkyl or tetra-alkyl ammonium salts. The pharmaceutically acceptable salts can also exist as various solvates, such as with water or ethanol, or the like. Mixtures of such solvates can also be prepared. In some embodiments, the proteinaceous compositions may be formulated into a neutral or salt form. Pharmaceutically acceptable salts, include acid addition salts (formed with the free amino groups of the protein) and which are formed with inorganic acids such as, for example, hydrochloric or phosphoric acids, or such organic acids as acetic, oxalic, tartaric, mandelic, or the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine or the like.

In some embodiments, the particles can be suspended in a non-aqueous liquid carrier, thereby forming a non-aqueous pharmaceutically acceptable composition. Importantly, the process of generating non-aqueous compositions with at least one therapeutic biologic does not significantly alter the structure or bioactivity of the biologic as described herein. In some embodiments, the liquid carrier is non-aqueous. In certain embodiments, the non-aqueous liquid carrier is an organic solvent. In some embodiments, the non-aqueous liquid carrier comprises at least two organic solvents.

In some embodiments, the organic solvent comprises benzyl benzoate, coconut oil, cottonseed oil, fish oil, grape seed oil, hazelnut oil, hydrogenated vegetable oils, olive oil, palm seed oil, peanut oil, peppermint oil, safflower oil, sesame oil, soybean oil, sunflower oil, walnut oil, corn oil, acetone, ethyl acetate, ethyl lactate, dimethylacetamide, dimethyl isosorbide, dimethyl sulfoxide, glycofurol, diglyme, methyl tert-butyl ether, N-methyl pyrrolidone, perfluorodecalin, polyethylene glycol, 2-pyrrolidone, tetrahydrofurfuryl alcohol, diglycerides, trigylcerides, medium-chain triglycerides (MCTs), caproic acid, caprylic acid, capric acid, lauric acid, ethyl laureate, triglycerides of the fractionated plant fatty acids C8 and C10, propylene glycol diesters of saturated plant fatty acids C8 and C10 (PGD), ethyl oleate, ethyl caprate, dibutyl adipate, fatty acid esters, hexanoic acid, octanoic acid, triacetin, diethyl glycol monoether, gamma-butyrolactone, eugenol, clove bud oil, citral, limonene, polyoxyl 40 hydrogenated castor oil, polyoxyl 35 castor oil, simple alcohols such as ethanol, octanol, hexanol, decanol, propanol, butanol, gamma-butyrolactone, tocopherol, octa-fluoropropane, (perfluorohexyl)octane, n-acetyltryptophan, ethyl laurate, methyl caprylate, ethyl caprylate, methyl caprate, methyl myristate, methyl oleate, methyl linoleate, dimethyl adipate, dibutyl suberate, diethyl sebacate, ethyl macadamiate, trimethylolpropane triisosterate, isopropyl laurate, isopropyl myristate, diethyl succinate, polysorbate esters, ethanol amine, propanoic acid, citral, anisole, anethol, benzaldehyde, linalool, caprolactone, phenol, thioglycerol, dimethylacetamide, diethylene glycol monoethyl ether, solketal, isosorbide dimethyl ether, ethyl formate, ethyl hexyl acetate, propylene glycol dicaprylate, caprylic triglyceride, ethyl linoleate, ethyl linolenate, or a combination thereof. Medium-chain triglycerides (MCTs) contain fatty acids that have a chain length of 6-12 carbon atoms. They include caproic acid (C6), caprylic acid (C8), capric acid (C10), and lauric acid (C12). In some embodiments, the organic solvent is ethyl oleate, diglycerides, trigylcerides, miglyol, ethyl macadamiate, ethyl caprate, diethyl succinate, diethylene glycol monoethyl ether, propylene glycol dicaprylate, caprylic triglyceride, ethyl linoleate, ethyl linolenate, medium-chain triglycerides (MCTs), sesame oil, propylene glycol diesters of saturated plant fatty acids C8 and C10 (PGD), triacetin, or a combination thereof. In certain embodiments, the organic solvent is ethyl oleate, ethyl caprylate, propylene glycol dicaprylate, diglycerides, trigylcerides, sesame oil, propylene glycol diesters of saturated plant fatty acids C8 and C10 (PGD), triacetin, or a combination thereof. In some embodiments, the organic solvent is ethyl oleate and sesame oil.

In some embodiments, a concentration of the therapeutic biologic in the compositions disclosed herein is greater than about 250 mg/mL, e.g., greater than about 275, 300, 325, 350, 375, 400, 425, 450, 475, 500, 525, 550, 575, 600, 625, 650, 675, 700, 725, 750, 775, or 800 mg/mL. In particular embodiments, the concentration of the therapeutic biologic in the composition is greater than about 800 mg/mL. In some embodiments, the concentration of the therapeutic biologic in the composition is greater than about 400 mg/mL. In certain embodiments, the concentration of the therapeutic biologic in the composition is greater than about 500 mg/mL. In some embodiments, the concentration of the therapeutic biologic in the composition is greater than about 600 mg/mL. In certain embodiments, the concentration of the therapeutic biologic in the composition is greater than about 700 mg/mL.

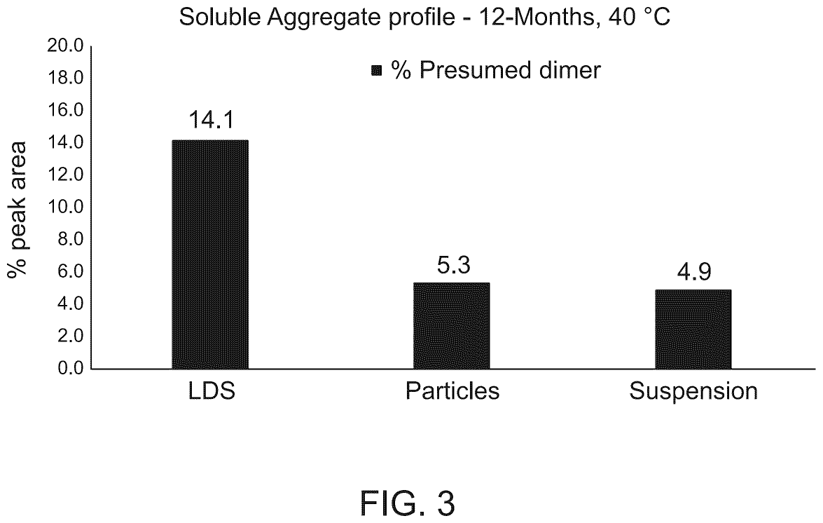

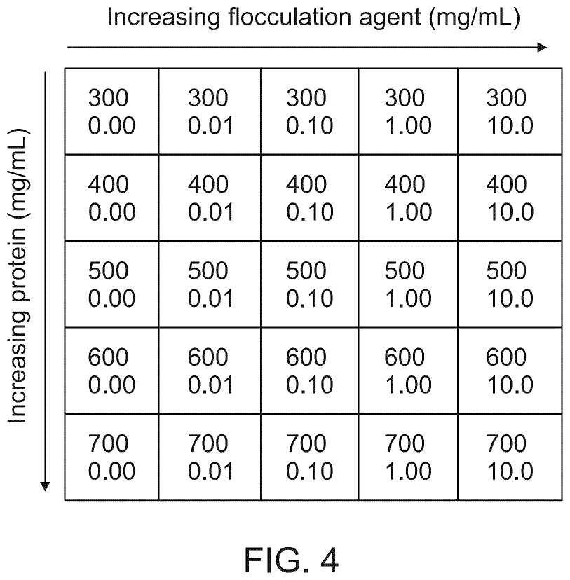

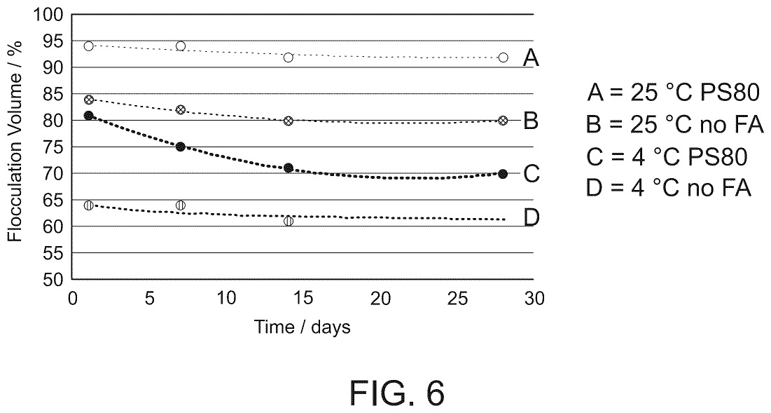

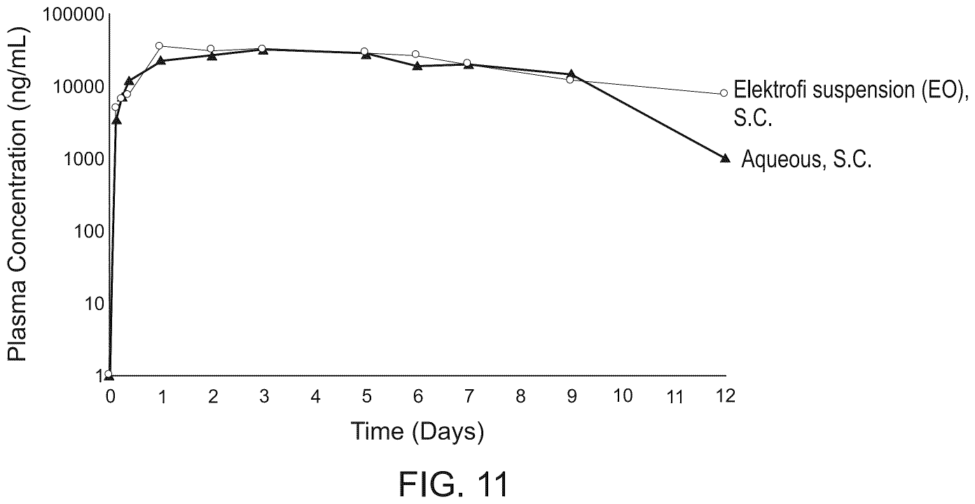

In a composition as described herein, the process of agglomeration can be controlled by the use of flocculation agents which can permit administration of particle compositions without the need for manual agitation or premixing prior to injection or administration. A flocculation agent can be non-ionic or ionic. Generally, an ionic flocculation agent interacts with charged particles, e.g., anionic flocculation agent interacts with positively charged particles, and a cationic flocculation agent interacts with negatively charged particles. In some embodiments, the flocculation agent is ionic. In some embodiments, the ionic flocculation agent is magnesium stearate, sodium dodecyl sulfate, sodium stearate, cetyltrimethylammonium bromide, lecithin, or a combination thereof. In particular embodiments, the flocculation agent is non-ionic. As disclosed herein, a non-ionic flocculation agent interacts with charged particles. In certain embodiments, the non-ionic flocculation agent is a polysorbate (polysorbate 80, polysorbate 60, polysorbate 20, e.g., Tween 80, Tween 60, Tween 20), an alkylphenol ethoxylate, glycerol, polyoxyethylated castor oil, docusate, decyl glucoside, nonoxynol-9, a sorbitan ester, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof. In some embodiments, the non-ionic flocculation agent is a polysorbate, an alkylphenol ethoxylate, a sorbitan ester, a poloxamer, or a combination thereof. In certain embodiments, the non-ionic flocculation agent is a polysorbate or a sorbitan ester. In some embodiments, the addition of a flocculation agent to the particle suspension does not substantially increase viscosity of the composition. As disclosed herein, the addition of a flocculation agent to a suspension of particles increases the flocculation volume of the suspension of the particles (see, e.g., FIG. 4 and FIG. 6). In certain embodiments, the addition of a flocculation agent to a suspension of particles increases the stability of the flocculation volume for at least one month (see, e.g., Example 6). In some embodiments, the addition of a flocculation agent to a suspension of particles reduces the syringe force (see, e.g., Examples 16 and 17). In some embodiments, the addition of a flocculation agent to a suspension of particles improves the pharmacokinetics (PK) of administration (e.g., subcutaneous administration) of microparticle (e.g., Ab microparticle) suspensions and shows higher bioavailability than the administration (e.g., subcutaneous administration) of aqueous Ab solution (see, e.g., FIG. 11). In some embodiments, the addition of a flocculation agent to a suspension of particles increases the stability of the proteins in the particles (see, e.g., FIG. 3).

In some embodiments, the composition described herein, use a concentration of the flocculation agent in the composition of less than about 50 mg/mL, e.g., less than about 45, 40, 35, 30, 25, 20, 15, 10, 5, 3, 1, 0.5, 0.1, 0.05, or 0.01 mg/mL. In some embodiments, the concentration of the flocculation agent in the composition is less than about 10 mg/mL. In some embodiments, the concentration of the flocculation agent in the composition is less than about 5 mg/mL. In some embodiments, the concentration of the flocculation agent in the composition is less than about 3 mg/mL. In particular embodiments, the concentration of the flocculation agent in the composition is less than about 1 mg/mL. In some embodiments, the concentration of the flocculation agent in the composition is less than about 0.1 mg/mL. In certain embodiments, the concentration of the flocculation agent in the composition is less than about 0.01 mg/mL.

Viscosity can play an important role in the handling and administration of injectable products. For suspension products, high viscosities drug products may be difficult to deliver through a needle (e.g., 27-gauge needle) since it takes greater force to actuate the injection device, e.g., syringe, portable drug delivery injection device, or orally dosed liquid injector capsules. Alternatively, using a larger gauge needle or requiring longer injection times can reduce patient compliance to therapy.

The term “viscosity” is used to describe the property of a fluid acting to resist shearing flow. For the purposes of the present disclosure, viscosity can be determined using a rheometer, e.g., AR-G2 Rheometer (TA Instruments, USA), fitted with a cone and plate (2°/40 mm) at 25° C. at a specified shear rate. In certain embodiments, the viscosity is measured at a shear rate in the Newtonian regime. In some embodiments, the viscosity is measured at a shear rate of 100 s⁻¹ or greater, e.g., at 1000 s⁻¹ or greater than 1000 s⁻¹, or greater than 10,000 s⁻¹. The term “low viscosity” as used herein, describes a composition, e.g., a liquid carrier, having a viscosity of less than about 100 mPa·s.

In some embodiments, the composition has a viscosity of less than about 200 mPa·s, less than about 150 mPa·s, less than about 125 mPa·s, less than about 100 mPa·s, less than about 95 mPa·s, less than about 90 mPa·s, less than about 85 mPa·s, less than about 80 mPa·s, less than about 75 mPa·s, less than about 70 mPa·s, less than about 65 mPa·s, less than about 60 mPa·s, less than about 55 mPa·s, less than about 50 mPa·s, less than about 45 mPa·s, less than about 40 mPa·s, less than about 35 mPa·s, less than about 30 mPa·s, less than about 25 mPa·s, less than about 20 mPa·s, less than about 19 mPa·s, less than about 18 mPa·s, less than about 17 mPa·s, less than about 16 mPa·s, less than about 15 mPa·s, less than about 14 mPa·s, less than about 13 mPa·s, less than about 12 mPa·s, less than about 11 mPa·s, less than about 10 mPa·s, less than about 9.5 mPa·s, less than about 9 mPa·s, less than about 8.5 mPa·s, less than about 8 mPa·s, less than about 7.5 mPa·s, less than about 7 mPa·s, less than about 6.5 mPa·s, less than about 6 mPa·s, less than about 5.5 mPa·s, or less than about 5 mPa·s (one millipascal-second). Mixtures of liquids may also be used to control viscosity. The units “mPa·s” and “cP” are used herein interchangeably in the broadest sense.

In some embodiments, the composition has a viscosity of less than about 100 mPa·s. In some embodiments, the composition has a viscosity of less than about 80 mPa·s. In certain embodiments, the composition has a viscosity of less than about 50 mPa·s. In some embodiments, the composition has a viscosity of less than about 40 mPa·s. In certain embodiments, the composition has a viscosity of less than about 30 mPa·s. In particular embodiments, the composition has a viscosity of less than about 25 mPa·s. In some embodiments, the composition has a viscosity of less than about 20 mPa·s. In certain embodiments, the composition has a viscosity of less than about 15 mPa·s. In some embodiments, the composition has a viscosity of less than about 10 mPa·s. In certain embodiments, the composition has a viscosity of less than about 5 mPa·s.

As described herein, a higher flocculation volume indicates increased agglomerates or less particle sedimentation, and accordingly, improved injection force performance due to more uniform dispersion of the particles in the liquid carrier. In some embodiments, the composition has a flocculation volume greater than about 50% after initial mixing, e.g., 60%, 70%, 75%, 80%, 85%, 90%, 95% or greater than about 98% after initial mixing. In some embodiments, the composition has a flocculation volume greater than about 70% after initial mixing. In certain embodiments, the composition has a flocculation volume greater than about 80% after initial mixing. In particular embodiments, the composition has a flocculation volume greater than about 85% after initial mixing. In certain embodiments, the composition has a flocculation volume of about 100% after initial mixing. Initial mixing refers to the homogenization of an initially heterogeneous particle suspension with a flocculating agent through bulk motion creating a flocculation volume.

In some embodiments, the flocculation volume is reduced by less than about 10%, e.g., 9%, 8%, 7%, 6%, 5%, 4%, 3%, 2%, or 1%, after at least one month under container closure storage conditions at less than about 40° C. In some embodiments, the flocculation volume is reduced by less than about 7% after at least one month under container closure storage conditions at less than about 40° C. In certain embodiments, the flocculation volume is reduced by less than about 5% after at least one month under container closure storage conditions at less than about 40° C. In some embodiments, the flocculation volume is reduced by less than about 3% after at least one month under container closure storage conditions at less than about 40° C. In certain embodiments, the flocculation volume is reduced by less than about 1% after at least one month under container closure storage conditions at less than about 40° C. In some embodiments, the plurality of particles and flocculation agent remain substantially suspended in the liquid carrier for at least one month. In some embodiments, the composition has substantially the same flocculation volume for at least one month.

In certain embodiments, insoluble particulate matter with characteristic sizes greater than or equal to about 100 m that persist upon dissolution in an aqueous liquid are referred to as Visible Particles (VP). In particular embodiments of the disclosure described herein, the composition is substantially free of Visible Particles (VP). In some embodiments, the aqueous liquid is water, aqueous buffer or a physiologically relevant aqueous liquid. As used herein, the term “physiologically relevant aqueous liquid” refers to any water-containing body fluid distributed in the extracellular compartment, e.g., extracellular fluid, interstitial fluid, intravascular fluid (blood, plasma, and lymph) and cerebrospinal fluid.

In some embodiments, insoluble particulate matter with characteristic sizes of about 1 μm to about 100 m that persist upon dissolution in an aqueous liquid are referred to as Subvisible Particles (SvPs). SvPs are present in quantities of about 0 to 100,000,000 per mL, e.g., about 0 to about 10,000,000 per mL, about 0 to about 1,000,000 per mL, about 0 to about 500,000 per mL, about 0 to about 100,000 per mL, about 0 to about 50,000 per mL, about 0 to about 10,000 per mL, about 0 to about 6,000 per mL, about 0 to about 1,000 per mL, about 0 to about 600 per mL, about 0 to about 250 per mL, about 0 to about 100 per mL, about 0 to about 60 per mL, or about 0 to about 10 per mL. In some embodiments, the count of particles with characteristic size greater than or equal to 10 m is about 0 to about 6,000 per mL, e.g., about 0 to about 1,000 per mL, about 0 to about 100 per mL, about 0 to about 10 per mL, about 0 to about 5 per mL, about 0 to about 3 per mL, or about 0 to about 1 per mL. In certain embodiments, the count of particles with characteristic size greater than or equal to 25 m is about 0 to about 600 per mL, e.g., about 0 to about 100 per mL, about 0 to about 10 per mL, about 0 to about 3 per mL, about 0 to about 1 per mL, about 0 to about 0.5 per mL, or about 0 to about 0.1 per mL. Exemplary methods of measuring SvPs include analysis of the therapeutic biologic with a Coulter Counter, HIAC Royco, or micro-flow imaging system after reconstitution and dilution of the therapeutic biologic to a standard concentration, e.g., about 100 mg/mL or about 1 mg/mL. In some embodiments, the composition has a concentration of insoluble Subvisible Particles (SvPs) of about 0 per mL to about 100,000,000 per mL of greater than about m particles upon dissolution in an aqueous liquid. In certain embodiments, the composition has a concentration of insoluble Subvisible Particles (SvPs) of about 0 per mL to about 6000 per mL of greater than about 10 m particles upon dissolution in an aqueous liquid. In particular embodiments, the composition has a concentration of insoluble Subvisible Particles (SvPs) of about 0 per mL to about 600 per mL of greater than about 25 m particles upon dissolution in an aqueous liquid. In certain embodiments, the composition is substantially free of insoluble Subvisible Particles (SvPs) upon dissolution in an aqueous liquid.

In certain embodiments, the composition has a concentration of insoluble Subvisible Particles (SvPs) having a characteristic size of greater than about 10 m of about 0 per mL to about 100,000,000 per mL upon dissolution in an aqueous liquid. In some embodiments, the composition has a concentration of insoluble Subvisible Particles (SvPs) having a characteristic size of greater than about 10 m of about 0 per mL to about 6000 per mL upon dissolution in an aqueous liquid. In some embodiments, the composition has a concentration of insoluble Subvisible Particles (SvPs) having a characteristic size of greater than about 25 m of about 0 per mL to about 600 per mL upon dissolution in an aqueous liquid. In particular embodiments, the aqueous liquid is water, aqueous buffer or a physiologically relevant aqueous liquid.

In some embodiments, insoluble particulate matter with characteristic sizes of about 100 nm to about 1 μm that persist upon dissolution in an aqueous liquid are referred to as submicron particles (SMP) and sometimes known as nanoparticles. The presence of such SMPs is thought to contribute to immunogenicity and thus should be avoided to minimize such effects. Quantitatively, SMPs are present in quantities of about 0 to 5×10¹² per mL, e.g., about 0 to about 0.5×10¹² per mL, about 0 to about 50×10⁹ per mL, about 0 to about 10×10⁹ per mL, about 0 to about 5×10⁹ per mL, about 0 to about 0.5×10⁹ per mL, about 0 to about 50×10⁶ per mL, about 0 to about 1×10⁶ per mL, about 0 to about 500,000 per mL, about 0 to about 200,000 per mL, about 0 to about 100,000 per mL, about 0 to about 10,000 per mL, about 0 to about 5000 per mL, or about 0 to about 1000 per mL. Exemplary methods of measuring SMPs quantitatively include analysis of the therapeutic biologic with a NanoSight, micro-flow imaging system, asymmetric field flow fractionation coupled to a multi-angle laser light scattering (AF4 MALS), Dynamic Light Scattering (DLS), or FLOWCAM™ imaging after reconstitution and dilution of the therapeutic biologic to a standard concentration, e.g., about 100 mg/mL, about 1 mg/mL, or about 1 μg/mL. Qualitatively, SMPs are within a range comparable to the starting monomeric therapeutic biologic solution. In particular embodiments, the composition is substantially free of submicron particles (SMP) upon dissolution in an aqueous liquid.

The present disclosure as described herein, concerns a highly concentrated composition comprising a flocculation agent and a plurality of particles suspended in a pharmaceutically acceptable liquid carrier, wherein the particles (e.g., substantially all of the particles) comprise at least one therapeutic biologic, wherein the composition upon dissolution in water, buffers or other physiologically relevant aqueous liquids, e.g., biological fluids in the patients' body, have a substantially similar immunogenicity compared to a similar aqueous composition comprising monomeric therapeutic biologics. As used herein, “physiologically relevant” conditions as may be encountered inside a mammal or human, can apply. The skilled artisan will be able to determine the set of conditions most appropriate for testing in accordance with the ultimate application of the compositions described herein. In some embodiments, the composition upon dissolution in an aqueous liquid has a substantially similar immunogenicity compared to an aqueous composition comprising at least one therapeutic biologic in soluble form, e.g., monomeric form. In particular embodiments, the composition is substantially non-immunogenic. In certain embodiments, the aqueous liquid is water, aqueous buffer or a physiologically relevant aqueous liquid.

As disclosed herein, the term “immunogenicity” refers to the induction of an immune response by an injected composition of the therapeutic biologic (the antigen), while “antigenicity” refers to the reaction of the composition of the therapeutic biologic with preexisting antibodies. Collectively, antigenicity and immunogenicity are referred to as “immunoreactivity”. In particular embodiments, the composition has substantially similar immunogenicity compared to an aqueous composition comprising at least one therapeutic biologic in soluble form, e.g., monomeric form. In certain embodiments, the composition is substantially non-immunogenic, for example, when subjected to non-immune response presence based on repeat-dose anti-drug antibody immunogenicity analysis (see, e.g., Example 8).

The ratio between toxicity and therapeutic effect for a particular composition, e.g., human IgG, is its therapeutic index and can be expressed as the ratio between LD₅₀ (the amount of compound lethal in 50% of the population) and ED₅₀ (the amount of compound effective in 50% of the population). Compositions that exhibit high therapeutic indices are preferred. Therapeutic index data obtained from cell culture assays and/or animal studies, e.g., human IgG, can be used in formulating a range of dosages for use in humans and known in the art. The dosage of such compounds preferably lies within a range of plasma concentrations that include the ED₅₀ with little or no toxicity. The dosage may vary within this range depending upon the dosage form employed and the route of administration utilized, e.g., subcutaneous injection. See, e.g., Fingl et al., In: THE PHARMACOLOGICAL BASIS OF THERAPEUTICS, Ch.1, p. 1, 1975. The exact composition, route of administration and dosage can be chosen by the individual physician in view of the patient's condition and the particular method in which the composition is delivered.

In some embodiments, the composition has substantially similar toxicity compared to an aqueous composition comprising at least one therapeutic biologic in soluble form, e.g., monomeric form. In some embodiments, the composition has reduced toxicity compared to an aqueous composition comprising at least one therapeutic biologic in soluble form, e.g., monomeric form. In particular embodiments, the composition is substantially non-toxic, for example, based on local tolerability and clinical observation analysis (see, e.g., Example 7).

In particular embodiments according to the disclosure as described herein, the particle composition has improved stability of the therapeutic biologic compared to an aqueous composition comprising at least one therapeutic biologic in soluble form, e.g., monomeric form (see, e.g., FIG. 3). A “stable” composition is one in which all the therapeutic biologic therein essentially retains their physical stability and/or chemical stability and/or biological activity upon storage, e.g., in a container closure, at the intended storage temperature, e.g., 4-40° C. It is desired that the composition essentially retains its physical and chemical stability, as well as its biological activity upon storage. The storage period is generally selected based on the intended shelf-life of the composition. Furthermore, the composition should be stable following freezing (to, e.g., −70° C.) and thawing of the composition, for example following 1, 2 or 3 cycles of freezing and thawing. Various analytical techniques for measuring protein stability are available in the art and are reviewed in Peptide and Protein Drug Delivery, 247-301, Vincent Lee Ed., Marcel Dekker, Inc., New York, N.Y, Pubs. (1991) and Jones, A. Adv. Drug Delivery Rev. 10: 29-90 (1993), for example. Stability can be measured at a selected temperature for a selected time period. Stability can be evaluated qualitatively and/or quantitatively in a variety of different ways, including evaluation of aggregate formation (for example using size exclusion chromatography, and/or by visual inspection); by assessing charge heterogeneity using cation exchange chromatography or capillary zone electrophoresis; amino-terminal or carboxy-terminal sequence analysis; mass spectrometric analysis; SDS-PAGE analysis to compare reduced and intact antibody; peptide map (for example tryptic or LYS—C) analysis; evaluating biological activity or antigen binding function of the antibody; etc. In some embodiments, the therapeutic biologic in the composition is stable for at least one month. In some embodiments, the therapeutic biologic in the composition is stable for at least two months. In certain embodiments, the therapeutic biologic in the composition is stable for at least three months. In certain embodiments, the therapeutic biologic in the composition is stable for at least three months at 40° C.

In some embodiments, the methods disclosed herein further comprise administering a pharmaceutically effective amount of at least one hyaluronan degrading agent that can be administered simultaneously, sequentially or intermittently with the composition. The subcutaneous tissue or the extracellular matrix is comprised of a network of fibrous proteins embedded within a viscoelastic gel of glycosaminoglycans. Hyaluronan is the prominent glycosaminoglycan of the subcutaneous tissue. Hyaluronan is secreted into the interstitium by fibroblasts as a viscous polymer that is subsequently degraded locally, in the lymph, through the action of hyaluronidases. Glycosaminoglycans are complex linear polysaccharides of the extracellular matrix and are characterized by repeating disaccharide structures of an N-substituted hexosamine and a uronic acid, as in the case of hyaluronan.

The hyaluronan degrading agent can enhance the subcutaneous administration of the composition comprising a plurality of particles and a flocculation agent, for example, by enhancing and/or increasing the volume of the composition being administered by injection thereby improving the absorption of the therapeutic biologic. The use of a hyaluronan degrading agent, such as hyaluronidase, can improve the subcutaneous administration of the therapeutic biologic into systemic circulation via the reversible hydrolyzation of hyaluronan, e.g., the reversible degradation of hyaluronan. The degradation of hyaluronan in the extracellular matrix temporarily opens channels in the subcutaneous tissue thereby allowing for larger volumes to be administered safely and comfortably into the subcutaneous tissue (see, e.g., Example 9). Moreover, the degradation of hyaluronan temporarily decreases the viscosity of the subcutaneous tissue and promotes the dispersion of injected liquids facilitating their absorption. The effects of hyaluronidase are local and reversible with complete reconstitution of the hyaluronan tissue occurring within 24 to 48 hours. See, e.g., Frost, G. I., “Recombinant human hyaluronidase (rHuPH20): an enabling platform for subcutaneous drug and fluid administration”, Expert Opinion on Drug Delivery, 2007; 4:427-440]. The increase in the permeability of the subcutaneous tissue through the degradation of hyaluronan correlates with the efficacy of hyaluronidase for their capability to increase the dispersion and absorption of compositions comprising a plurality of particles administered simultaneously, sequentially or intermittently. In certain embodiments, the methods described herein, further comprise administering at least one hyaluronan degrading agent.

In some embodiments, the methods of the disclosure further comprise administering at least one hyaluronan degrading agent, e.g., a pharmaceutically effective amount of at least one hyaluronan degrading agent that is administered simultaneously, sequentially or intermittently with the composition. In certain embodiments, the hyaluronan degrading agent is a hyaluronidase enzyme, also referred to herein as a hyaluronidase. In some embodiments, the hyaluronidase is a soluble neutral-active hyaluronidase. In certain embodiments, the hyaluronidase is a mammalian hyaluronidase. In particular embodiments, the mammalian hyaluronidase is a human hyaluronidase. In particular embodiments, the human hyaluronidase is a recombinant human hyaluronidase, e.g., a rHuPH20. Certain recombinant human hyaluronidases that are suitable for use in the compositions disclosed herein are commercially available, e.g., rHuPH20 from Halozyme Therapeutics (San Diego, CA).

According to certain embodiments of the disclosure, the hyaluronan degrading agent can be administered simultaneously, sequentially or intermittently with the composition. As used herein, the term “simultaneous” or “near-simultaneous” administration refers to components that are administered at the same time, e.g., the simultaneous administration of both the composition and hyaluronan degrading agent. In some embodiments, the hyaluronan degrading agent and composition are administered in a single composition, e.g., co-formulated. In some embodiments, the hyaluronan degrading agent and composition are administered as separate formulations. However, one component could be administered within a few minutes or hours, for example, at the same medical appointment or doctor's visit. Such administration is referred to as “sequential” administration. In certain embodiments, sequential administration refers to the sequential administration of the hyaluronan degrading agent and the composition as described herein. In particular embodiments, the hyaluronan degrading agent is administered first, followed by administration of one or more doses of the composition. In certain embodiments, the components may be administered intermittently as co-formulations or separate formulations. In particular embodiments, the hyaluronan degrading agent is administered first.

### Methods of the Disclosure

Where a clinical application of a therapeutic composition comprising a therapeutic biologic, e.g., mAb, is undertaken, it will generally be beneficial to prepare a pharmaceutical or therapeutic composition that is appropriate for the intended application. In certain embodiments, pharmaceutical compositions may comprise, for example, at least about 0.1% of an active therapeutic biologic. In some embodiments, an active therapeutic biologic may comprise about 2% to about 99% of the weight of the unit, or about 50% to about 99%, for example, and any range derivable therein.

As described herein, CD20 (also known as Bp35) is a B-lymphocyte-restricted differentiation antigen that is expressed during early pre-B-cell development and remains until plasma cell differentiation. CD20 can be a useful target for B-cell lymphomas as this antigen is expressed at very high densities on the surface of malignant B-cells, i.e., B-cells wherein unabated proliferation can lead to B-cell lymphomas. The Food and Drug Administration (FDA) has approved the therapeutic use of an anti-CD20 antibody, rituximab (RITUXAN®), for use in relapsed and previously treated low-grade non-Hodgkin's lymphoma (NHL). Rituximab acts by binding to the CD20 antigen on B-cells which results in the lysis of the B-cell by a mechanism thought to involve complement-dependent cytotoxicity (CDC) and antibody-dependent cell mediated cytotoxicity (ADCC). In certain embodiments, the therapeutic biologic is an antibody. In particular embodiments, the antibody is an anti-CD20 antibody.

Provided herein are compositions and methods for treating a disease or condition in a subject in need thereof; comprising administering to the subject a pharmaceutically effective amount of a composition comprising: a plurality of particles suspended in a pharmaceutically acceptable liquid carrier, wherein the particles (e.g., substantially all of the particles) comprise at least one therapeutic biologic or a salt thereof; and a flocculation agent, wherein the concentration of the flocculation agent in the composition is less than about 50 mg/mL. In some embodiments, the concentration of the therapeutic biologic or salt thereof in the composition is greater than about 250 mg/mL. In certain embodiments, provided herein, are methods for treating cancer, inflammatory disease or an immune disease in a subject in need thereof; comprising administering to the subject a pharmaceutically effective amount of a composition comprising: a plurality of particles suspended in a pharmaceutically acceptable liquid carrier, wherein the particles (e.g., substantially all of the particles) comprise at least one therapeutic biologic or a salt thereof; and a flocculation agent, wherein the concentration of the flocculation agent in the composition is less than about 50 mg/mL; and wherein the concentration of the therapeutic biologic or salt thereof in the composition is greater than about 250 mg/mL. Also provided herein, are methods for administering a pharmaceutically effective composition comprising: a plurality of particles suspended in a pharmaceutically acceptable liquid carrier, wherein the particles (e.g., substantially all of the particles) comprise at least one therapeutic biologic or a salt thereof; and a flocculation agent, wherein the concentration of the flocculation agent in the composition is less than about 50 mg/mL; and wherein the concentration of the therapeutic biologic or salt thereof in the composition is greater than about 250 mg/mL.

The term “treat” or “treating” or “treatment” generally refers to therapeutic treatment measures, e.g., treating, reversing and/or down-regulating a disease or condition. As used herein, “treat” or “treating” means to administer a therapeutic biologic, such as a composition containing any of the antibodies or antigen binding fragments thereof of the present disclosure, internally or externally to a subject having one or more disease symptoms, or being suspected of having a disease, for which the biologic has therapeutic activity or prophylactic activity. Typically, the therapeutic biologic is administered in an amount effective to alleviate one or more disease symptoms in the treated subject or population, whether by inducing the regression of or inhibiting the progression of such symptom(s) by any clinically measurable degree. The amount of a therapeutic biologic that is effective to alleviate any particular disease symptom may vary according to factors such as the disease state, age, and weight of the subject or patient, and the ability of the therapeutic biologic to elicit a desired response in the subject or patient. Whether a disease symptom has been alleviated can be assessed by any clinical measurement typically used by physicians or other skilled healthcare providers to assess the severity or progression status of the symptom. The term further includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a human or animal subject with a disorder, disease or symptom, or with the potential to develop such a disorder, disease or symptom. As used herein, “treatment,” as it applies to a human or veterinary subject, refers to therapeutic treatment, as well as diagnostic applications. “Treatment” as it applies to a human or veterinary subject, encompasses contact of the therapeutic biologic, e.g., antibodies or antigen binding fragments of the present disclosure to a human or animal subject. Subjects requiring treatment for cancer include those already having a benign, pre-cancerous, or non-metastatic tumor as well as those in which the occurrence or recurrence of cancer is to be prevented. In some embodiments, the objective or outcome of treating or treatment may be to reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. In some embodiments, the efficacy of treatment can be measured by assessing the duration of survival, time to disease progression, the response rates (RR), duration of response, and/or quality of life. The term “prevent” or “preventing” or “prevention” as used herein refers to any action that inhibits or delays the onset of a disease or condition in a subject in need thereof; comprising administering to the subject a pharmaceutically effective amount of a composition according to the present disclosure. In certain embodiments of the disclosure, “treating” a subject afflicted with a disease or condition shall include, without limitation, (i) slowing, stopping or reversing the progression of the disease or condition, (ii) slowing, stopping or reversing the progression of the symptoms of the disease or condition, (iii) reducing the likelihood of the recurrence of the disease or condition, and/or (iv) reducing the likelihood that the symptoms of the disease or condition will recur.

Non-limiting examples of cancer which can be treated by the compositions and methods described herein, include but are not limited to any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B-cell, Burkitt, cutaneous T cell, histiocytic, lymphoblastic, T cell, thymic, B-cell non-Hodgkin lymphoma), gliomas, adenocarcinoma, adrenal tumor, hereditary adrenocortical carcinoma, brain malignancy (tumor), various other carcinomas (e.g., bronchogenic large cell, ductal, malignant ascites, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B-cell), hypernephroma, insulinoma, islet tumor, keratoma, leiomyoblastoma, leiomyosarcoma, leukemia (e.g., acute lymphatic, acute lymphoblastic, acute lymphoblastic pre-B-cell, acute lymphoblastic T cell leukemia, acute-megakaryoblastic, monocytic, acute myelogenous, acute myeloid, acute myeloid with eosinophilia, B-cell, basophilic, chronic myeloid, chronic, B-cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B-cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), osteoporosis, bone metastasis, papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, or Turcot syndrome with glioblastoma.

In some embodiments, the therapeutic biologic is an immunotherapy. In some embodiments, the immunotherapy is an anti-CD20 antibody. In certain embodiments, the anti-CD20 antibody is rituximab. In particular embodiments of the compositions and methods described herein, may be useful for the treatment of non-Hodgkin's lymphoma (NHL) in a subject in need thereof; comprising administering to the subject a pharmaceutically effective amount of a composition comprising: a plurality of particles suspended in a pharmaceutically acceptable liquid carrier, wherein the particles (e.g., substantially all of the particles) comprise at least one therapeutic biologic or a salt thereof; and a flocculation agent, wherein the concentration of the flocculation agent in the composition is less than about 50 mg/mL; and wherein the concentration of the therapeutic biologic or salt thereof in the composition is greater than about 250 mg/mL. As described herein, any antibody capable of binding the CD20 antigen may be used in the methods of the instant disclosure. Antibodies which bind the CD20 antigen include, for example: C2B8 (rituximab; RITUXAN®) (U.S. Pat. No. 5,736,137, expressly incorporated herein by reference); the yttrium-[90]-labeled 2138 murine antibody designated Y2B8 (U.S. Pat. No. 5,736,137, expressly incorporated herein by reference); murine IgG2a 131 optionally labeled with 131 1 to generate the 131 1-B1 antibody (BEXXAR®) (U.S. Pat. No. 5,595,721, expressly incorporated herein by reference); murine monoclonal antibody 1F5 (Press et al. Blood 69(2): 584-591 (1987)); chimeric 2H7 antibody (U.S. Pat. No. 5,677,180 expressly incorporated herein by reference); and monoclonal antibodies L27, G28-2, 93-1 133, B—Cl or NU—B2 available from the International Leukocyte Typing Workshop (Valentine et al., In: Leukocyte TypingIII (McMichael, Ed., p. 440, Oxford University Press (1987)).

In certain embodiments of the disclosure, the anti-CD20 antibody is rituximab. Rituximab is a genetically engineered chimeric murine/human monoclonal antibody. Rituximab is an IgG, kappa immunoglobulin containing murine light and heavy chain variable region sequences and human constant region sequences. Rituximab has a binding affinity for the CD20 antigen of approximately 8.0 nM and is commercially available, e.g., from Genentech (South San Francisco, CA).

In other embodiments, the antibody is trastuzumab (Herceptin; Genentech, San Francisco, CA). Trastuzumab is a humanized monoclonal antibody that binds to the extracellular portion of the HER2 ectodomain, preventing dimerization and the cascade that leads to the expression of growth factors. In certain embodiments, trastuzumab is used to treat breast cancer esophageal cancer, stomach cancer, or other HER2-overexpressing new or metastatic cancers.

Also provided herein are methods for treating an inflammatory disease in a subject in need thereof; comprising administering to the subject a pharmaceutically effective amount of a composition comprising: a plurality of particles suspended in a pharmaceutically acceptable liquid carrier, wherein the particles (e.g., substantially all of the particles) comprise at least one therapeutic biologic or a salt thereof; and a flocculation agent, wherein the concentration of the flocculation agent in the composition is less than about 50 mg/mL; and wherein the concentration of the therapeutic biologic or salt thereof in the composition is greater than about 250 mg/mL.

In some embodiments of the methods described herein, the inflammatory disease includes but are not limited to a joint disease, an ophthalmic disease, retinal disease, Crohn's disease, irritable bowel syndrome, Sjogren's disease, tissue graft rejection, asthma, multiple sclerosis, scleroderma, Goodpasture's syndrome, atherosclerosis, chronic idiopathic thrombocytopenic purpura, Addison's disease, Parkinson's disease, Alzheimer's disease, diabetes, septic shock, myasthenia gravis, inflammatory pelvic disease, inflammatory bowel disease, urethritis, uveitis, sinusitis, pneumonitis, encephalitis, meningitis, myocarditis, osteomyelitis, myositis, hepatitis, gastritis, enteritis, appendicitis, pancreatitis, or cholocystitis.

In some embodiments, the compositions and methods described herein, are useful for treating immune disease, e.g., acquired hypogammaglobulinemia secondary to hematological malignancies, chronic inflammatory demyelinating polyneuropathy (CIDP), Guillain-Barre Syndrome, Idiopathic thrombocytopenic purpura, inflammatory myopathies, Lambert-Eaton myasthenic syndrome, multifocal motor neuropathy, Myasthenia Gravis, Moersch-Woltmann syndrome, secondary hypogammaglobulinaemia specific antibody deficiency, Acute disseminated encephalomyelitis, Autoimmune haemolytic anemia; Cicatricial pemphigoid, Evans syndrome, Foeto-maternal/neonatal alloimmune thrombocytopenia (FMAIT/NAIT), Haemophagocytic syndrome, high-risk allogeneic haemopoietic stem cell transplantation, IgM paraproteinaemic neuropathy, kidney transplantation, multiple sclerosis, Opsoclonus myoclonus ataxia, Post-transfusion purpura, Toxic epidermal necrolysis/Steven Johnson syndrome (TEN/SJS), Toxic shock syndrome, Alzheimer's Disease, multiple myeloma, sepsis; B-cell tumors, trauma, or a bacterial, viral, fungal infection. In some embodiments, the immune disease is an autoimmune disease. In certain embodiments, the autoimmune disease include but are not limited to multiple sclerosis, scleroderma, type-I diabetes, rheumatoid arthritis, thyroiditis, Reynaud's syndrome, Sjorgen's syndrome, autoimmune uveitis, autoimmune myocarditis, inflammatory bowel disease, amyotrophic lateral sclerosis (ALS), systemic lupus, neuromyelitis optica, idiopathic thrombocytopenic purpura, myasthenia gravis, ulcerative colitis, Crohn's disease, polyarthritis, graft-versus-host reactions, juvenile-onset diabetes, Hashimoto's thyroiditis, Grave's disease, pernicious anemia, chronic active (lupoid) hepatitis, psoriatic arthritis, or neurodermatitis. By “decrease” or “reduce” is meant becoming less or smaller, as in number, amount, size, or intensity. In certain embodiments, decreasing the risk of a disease (e.g., such as focal segmental glomerulosclerosis (FSGS)) includes a decrease in the likelihood of developing the disease by at least about 20%, for example by at least about 30%, 40%, 50%, 60%, 70%, 80%, or 90%. In some embodiments, decreasing the risk of a disease includes a delay in the development of the disease, for example a delay of at least about six months, such as about one year, such as about two years, about five years, or about ten years.

As described herein, particle-based compositions with flocculation agents can directly address the current challenges of subcutaneous administration of therapeutic biologics, e.g., mAb, by enabling injectability with low injection forces without the need for resuspension prior to injection. In some embodiments, the particles and flocculation agent are suspended in a non-aqueous liquid carrier and remain substantially suspended in the liquid carrier for at least one month, eliminating the need for complex, time-consuming resuspension procedures. The compositions described herein, can be used in a prefilled syringe, prefilled portable drug delivery injection device, or prefilled orally dosed liquid injector capsules. The highly dispersible nature of the particles in the composition that are described herein, allows for a patient-friendly subcutaneous injection. Within the subcutaneous space, the therapeutic biologics comprised in the particles, as described herein, readily return to their original monomeric state upon injection, enabling full bioavailability. In particular embodiments, the compositions do not compromise the therapeutic biologic quality and achieve higher loading, thus, allowing the therapeutic biologic, e.g., mAbs, to be delivered easily by subcutaneous injection to treat a disease or condition in a subject in need thereof; wherein the disease or condition is cancer, inflammatory disease or an immune disease. In some embodiments, the disease or condition is cancer. In some embodiments, the disease or condition is inflammatory disease or condition. In certain embodiments, the disease or condition is an immune disease.

The pharmaceutical composition and methods as disclosed herein, can be administered to a subject by any suitable route of administration including, for example, parenterally or intraperitoneally. In some embodiments, the composition is administered by parenteral, subcutaneous, oral, epidermal, intradermal, intramuscular, interarterial, intraperitoneal, or intravenous injection. In particular embodiments, the composition is administered by subcutaneous injection. Details of appropriate routes of administration and compositions suitable for same can be found in, for example, U.S. Pat. Nos. 6,110,973; 5,763,493; 5,731,000; 5,541,231; 5,427,798; 5,358,970 and 4,172,896, as well as in patents cited therein. The term “pharmaceutical composition” as disclosed herein, refers to a preparation which is in such form as to permit a therapeutic biologic in the composition to be effective, e.g., when administered to a subject, and which contains no additional components which are unacceptably toxic to a subject to which the composition would be administered. Such compositions are sterile. A “sterile” composition or formulation is aseptic or free from all living microorganisms and their spores.

The term “injectability” or “syringeability”, refers to the relative ease with which a liquid composition, as described herein, can be administered to a subject through the use of an injection device, e.g., syringe, portable drug delivery injection device, or orally dosed liquid injector capsules. In particular, injectability is influenced in part by the viscosity of the composition and more importantly by the sedimentation of the particles, the injection or transfer flow rate, and the needle characteristics, e.g., length and gauge. In some embodiments, the injectability is determined by measuring the viscosity of the composition at various shear rates. In some embodiments, the injectability is determined by measuring the breakaway and/or glide forces required to actuate an injection device consisting of a barrel, a plunger and a needle. In certain embodiments, the barrel of the syringe has an inner diameter of at least 6 mm. As described herein, the injectability of the composition comprising a flocculation agent and a plurality of particles comprising at least one therapeutic biologic, is superior to that of an aqueous formulation with about the same concentration of aqueous monomeric therapeutic biologics. The term “injectability” or “syringeability”, can also refer to the injection performance of a pharmaceutical composition through a syringe equipped with a 16-33-gauge needle, optionally, thin walled or ultra-thin walled (UTW) needle. In certain embodiments, the syringe is equipped with a needle that is at least 8 mm in length. Injectability depends upon factors such as pressure or force required for injection, evenness of flow, aspiration qualities, and freedom from clogging. As described herein, injectability may be assessed by comparing the injection force of the composition after a period of time, to a standard particle composition without added flocculation agents. The compositions as described herein, can improve injectability after a period of time, with the injection force reduced by at least 10%, preferably by at least 30%, more preferably by at least 50%, and most preferably by at least 75% when compared to a standard particle composition having the same concentration of therapeutic biologic under otherwise the same conditions. Alternatively, injectability of the compositions can be assessed by comparing the time or injection force required to inject the flocculation volume, such as about 2.0 mL, preferably about 1.5 mL, more preferably about 1.0 mL, and most preferably about 0.5 mL, of compositions when the syringe is depressed with the same force, over a period of time, e.g., sedimentation of the particles over a period of time. The phrase “flow rate” refers to the volume of liquid composition that may pass through a given cross sectional area per unit time. In general, the flow rate formula is Q=A×v, where Q is the flow rate, A is the cross-sectional area at a point in the path of the flow, and v is the average velocity of the liquid at that point. In certain embodiments, the flow rate is constant. In particular embodiments, the flow rate is at least about 0.1 mL/sec.

The term “injection breakaway force” refers to the force required to overcome friction between the syringe barrel and plunger of a standard injection device before ejection of the contents of the syringe can take place at a steady rate, e.g., the maximum force required to break the static friction of the plunger. The force is applied at the outward-facing end of the syringe plunger shaft and directed along the axis of the syringe barrel. The contents of the syringe are ejected through a syringe needle of prescribed gauge and length. In certain embodiments, the injection breakaway force is measured through a load cell placed at the outward-facing end of the syringe plunger during actuation.

The terms “syringe force”, “injection force”, “injection glide force” or “glide force” are used interchangeably herein and refer to the force required to maintain a steady ejection of the contents of a standard injection device, e.g., the force required to maintain plunger movement once static friction has been overcome. The force is applied at the outward-facing end of the syringe plunger shaft and directed along the axis of the syringe barrel. The contents of the syringe are ejected through a syringe needle of prescribed gauge and length. The term “Newtonian regime” or “N” means a range of shear stress which are linearly proportional or nearly linearly proportional to the local strain rate at every point. In some embodiments, the addition of a flocculation agent to a suspension of particles reduces the syringe force (see, e.g., Examples 16 and 17).

The administering of the compositions described herein may comprise administering using a 18-33-gauge needle. The 18-33-gauge needle may have a length of about 19 mm (¾-inch) or less; or preferably about 13 mm (½-inch) or less. In some embodiments, the administering of the composition uses a 27-33-gauge needle. In some embodiments, the 27-33-gauge needle has a length of about 13 mm (½-inch) or less. In certain embodiments, the composition is dispensed from a needle having a gauge in the range of 18-gauge to 33-gauge, e.g., a 19, 20, 21, 22, 23, 24, 25, 26, 27, 28, 29, 30, 31, 32-gauge to 33-gauge. In some embodiments, the composition is dispensed from a needle having a gauge in the range of 27-gauge to 30-gauge. In certain embodiments, the composition is dispensed from a needle having a gauge in the range of 25-gauge to 27-gauge. In particular embodiments, the composition is dispensed from a needle having a gauge of 27-gauge.

In some embodiments, the composition is dispensed using an injection force of less than about 70 N, e.g., about 60, 50, 40, 30, 25, 20, 15, 10, or 5 N. In some embodiments, the composition is dispensed using an injection force of less than about 25 N. In particular embodiments, the composition is dispensed using an injection force of less than about 20 N. In certain embodiments, the composition is dispensed using an injection force of less than about 15 N. In some embodiments, the composition is dispensed using an injection force of less than about 10 N. In certain embodiments, the composition is dispensed using an injection force of less than about 5 N. In some embodiments, the injection force increases at a lower rate than the viscosity of the composition as the concentration of the therapeutic biologic in the composition is increased. In certain embodiments, the injection force remains substantially the same for at least one month under container closure storage conditions at less than about 40° C.

In other cases, the composition comprising a plurality of particles and a flocculation agent as described herein, optionally, further comprising administering at least one hyaluronan degrading agent, e.g., a hyaluronidase, that is administered simultaneously, sequentially or intermittently with the composition. In some embodiments, the composition to be administered is less than about 20.0 mL, e.g., 15.0, 10.0, 5.0, 2.0, 1.5, 1.0, 0.5 mL. In some embodiments, the composition to be administered is less than about 2.0 mL. In certain embodiments, the composition to be administered is less than about 1.5 mL. In some embodiments, the composition to be administered is less than about 1.0 mL. In particular embodiments, the composition to be administered is less than about 0.5 mL.

In some embodiments, the composition dissolves after administration in less than about 10, 9, 8, 7, 6, 5, 4, 3, 2, 1 min. In certain embodiments, the composition dissolves after administration in less than about 60 s, e.g., 59, 58, 57, 56, 55, 54, 53, 52, 51, 50, 49, . . . 10, 9, 8, 7, 6, 5, 4, 3, 2, 1 s. In particular embodiments, the composition immediately dissolves after administration.

The compositions described herein can be used for parenteral administration, e.g., formulated for injection via the intravenous, intramuscular, subcutaneous or even intraperitoneal routes, without the need for manual agitation or premixing of the injection device prior to use. The pharmaceutical compositions suitable for injectable use include sterile non-aqueous liquid carriers comprising sterile particles. In all cases the composition must be sterile and must be fluid to the extent that it can be easily injected. The pharmaceutical compositions also should be stable under the conditions of current good manufacture procedures (cGMP) and storage.

The present disclosure generally relates to compositions for use in a syringe or portable drug delivery injection devices. In particular embodiments, the composition is in the syringe or portable drug delivery injection device. For example, the syringes disclosed herein can be prefilled syringes. In certain embodiments, the syringe or portable drug delivery injection device is prefilled with the composition. Examples of portable drug delivery injection devices include, pen injectors, automatic injectors (e.g., autoinjectors, patches), or orally dosed liquid injector capsules (e.g., robotic pills that deliver drug in the gastrointestinal tract or the like). The compositions described herein, can be enclosed in disposable syringes, cartridges or any other container closures made of glass or plastic or the like. In some embodiments, the composition is administered by syringe injection. In some embodiments, the composition is dispensed from a prefilled syringe. In certain embodiments, the portable drug delivery injection device is configured to automatically, or semi-automatically, deliver a drug to a patient using a wireless communication system.

An automatic injector, e.g., autoinjector, pen injector or medication pen, or orally dosed liquid injector capsules, is a prefilled and pre-assembled injection device that enables parenteral administration of a therapeutic agent. An advantage of automatic injectors is that they contain a measured dose of a therapeutic agent in a sealed sterile cartridge or injection device. Automatic injectors are useful for people with low dexterity, poor vision, or who need portability to administer a therapeutic agent on time and can also decrease the fear or adversity towards self-injection of therapeutic agents, which increases the likelihood that a person takes the medication. Automatic injectors are most useful in emergency situations that allow quick and simple self-administration of the therapeutic agent without having to measure dosages.

Ensuring that the particle suspensions of therapeutic biologics in the compositions and methods disclosed herein be completely delivered is often important for particle suspensions for which injection forces may already be relatively high. At high shear, as is often the case for portable drug delivery injection devices, the problem of ensuring complete injection of a unit dose at high injection forces can be compounded by particle sedimentation in the composition making user operation challenging.

In automatic injectors, the particles of therapeutic biologics in the composition would likely be stored as a particle suspension which would be subsequently injected. However, long-term storage (e.g., 3 months to 12 months) of the particle suspension has drawbacks, such as particle sedimentation which can lead to high injection forces. These automatic injectors require that the user manually shake the injector body to expedite resuspension of the particles immediately prior to injection. Unfortunately, steps such as manually shaking the automatic injector, increase the time needed to administer a dose of the therapeutic agent, which is undesirable in many emergency medical situations where rapid delivery of the therapeutic agent is required.

In some embodiments, the composition is administered by a syringe or a portable drug delivery injection device. In particular embodiments, the composition is in the syringe or portable drug delivery injection device. In particular embodiments, the composition is administered by a syringe. In certain embodiments, the composition is administered by a portable drug delivery injection device. In certain embodiments, the portable drug delivery injection device is a pen injector. In some embodiments, the portable drug delivery injection device is an automatic injector. In certain embodiments, the portable drug delivery injection device is an orally dosed liquid injector capsule. In particular embodiments, the syringe or portable drug delivery injection device is prefilled with the composition. In some embodiments, the composition is administered in one or more doses. In some embodiments, the composition is administered in a single dose. In certain embodiments, the composition is administered in multiple doses.

In some embodiments, the composition is administered by a syringe. In some embodiments, the composition is administered by a pen injector. In certain embodiments, the composition is administered by an automatic injector. In some embodiments, the composition is administered by an orally dosed liquid injector capsule. In particular embodiments, the syringe, pen injector, automatic injector, orally dosed liquid injector capsule is prefilled.

The compositions of the present disclosure may be utilized to treat a disease or condition in a subject in need thereof. In some embodiments, the disease or condition is cancer. In some embodiments, the disease or condition is inflammatory disease or condition. In certain embodiments, the disease or condition is an immune disease. In certain embodiments, the subject is a mammal, e.g., human or animal. The compositions as described herein, can be administered to a mammal, e.g., human or animal subject in vivo using a variety of known routes and techniques. For example, the composition may be provided as an injectable suspension, and administered via parenteral, subcutaneous, oral, epidermal, intradermal, intramuscular, interarterial, intraperitoneal, or intravenous injection, using a conventional needle and syringe or portable drug delivery injection device. In certain embodiments, the composition is administered to a mammal. In particular embodiments, the composition is administered to a human.

In particular embodiments according to the disclosure, when the composition is administered, complete dissolution can occur immediately or within seconds, mitigating any immunological risks posed by the particles that are persisting in the subcutaneous space. For example, compared to an equal dose of aqueous monomeric mAbs, dosing of particles comprising mAb has shown to produce a similar or improved pharmacokinetic (PK) profile (AUC, Cmax and Tmax) as described herein. In certain embodiments, the composition immediately dissolves after administration.

The compositions described herein, demonstrate the subcutaneous delivery of high concentration therapeutic biologics (300-750 mg/mL) compositions without the loss of bioactivity. This has been achieved for a variety of therapeutic biologics by using an organic solvent, e.g., sesame oil, medium-chain triglycerides (MCTs), propylene glycol diesters of saturated plant fatty acids C8 and C10 (PGD), fatty acid esters (ethyl oleate (EO)) as the liquid carrier. For instance, ethyl oleate is a fatty acid ester with a viscosity of about 6 mPa·s at 25° C., and does not chemically interact with the particles during storage or in vivo dissolution.

The production of dense (1.32 g/cm³), round, particles with controllable size distribution (polydispersity index <0.2) can be accomplished using a variety of therapeutic biologics described herein. Focused Ion Beam Scanning Electron Microscopy (FIB-SEM) can be used to determine if the particles contain no void spaces which is vital for reaching high protein loadings in the composition and X-ray Photoelectron Spectroscopy (XPS) can be used to control the radial distribution of the particles. In certain embodiments, the particles size can be about 10 to about 80 m, which is preferable for reaching low viscosity compositions but small enough to prevent syringe clogging in a 27-gague needle. For example, by loading in a prefilled syringe, a flocculation agent and particles at about 400 mg/mL protein concentration in an ethyl oleate (EO) liquid carrier, a therapeutic composition can be formed with a viscosity of about 20 mPa·s (correlating to an injection force of about 4 N) which can be stored for at least one month without substantial change in flocculation volume, viscosity, or injection force (e.g., the particles remain substantially suspended in the liquid carrier for at least one month). See Examples 5 and 6. In some embodiments, the composition has substantially the same flocculation volume for at least one month. See Example 6. In some embodiments, the plurality of particles and flocculation agent remain substantially suspended in the liquid carrier for at least three months. See Examples 10 and 11. In certain embodiments, the injection force remains substantially the same for at least three months under container closure storage conditions at about 25° C. See Examples 10 and 11. In some embodiments, the yield stress remains substantially the same for at least one month under container closure storage conditions at about 25° C. See Example 13. In particular embodiments, the addition of a flocculation agent to the composition prevents the particles from sedimentation, preventing clogging of the needle.

As described herein, characterization of the structural stability of the particles in the composition was accomplished using size-exclusion chromatography (SEC), differential scanning fluorimetry (DSF), circular dichroism (CD), cation exchange chromatography (CIEX) and subvisible particle (SvP) analysis. In addition, preservation of bioactivity was analyzed using pharmacokinetics (PK) and immunogenicity studies. See Examples 7 and 8.

Also described herein, SEC data confirmed that minimal aggregate formation was observed upon processing as compared with the label formulation (aqueous mAb as an FDA approved formulation) containing aggregates compared to the reformulated particle composition which contained 96.6% monomer (protein content 507 mg/mL). See Example 11. After 30 days of storage at 40° C., DSF showed less than 1° C. thermal shift across the samples, and CD could not detect any differences in secondary structure (beta-sheet percentage). CIEX was used to analyze charged variants of proteins as mandated by regulation (ICH Q6B), to ensure that no chemical modifications occurred during the preparation described herein, and upon storage. In certain embodiments, the composition may be less prone to chemical modification upon storage than the FDA labeled formulation. This is due to the protein being more stable in the solid state as particles.

Bioactivity preservation has been demonstrated through flow cytometry assays after storage for 30 days at 40° C. No discernable difference was evident between FDA label formulations and those compositions as described herein. As shown in Example 4 and Example 5 herein, in the case of FDA label formulation, bioactivity decreased significantly after storage, whereas for the particles in the composition, no decrease in activity was observed.

Prior to in vivo studies commencing, the particles used for the composition were obtained under substantially aseptic conditions. Bacterial endotoxin levels were about 3 orders of magnitude below accepted injection standards (0.05 EU/mg to 0.25 EU/mg). In addition, a microbial growth assay indicated no observable growth.

The compositions described herein, are comparable to aqueous FDA label formulations in terms of rat pharmacokinetics (PK), SC clearance (mouse) and efficacy (mouse xenograft). The PK profiles are shown in Example 7, the mAb microparticle suspension (SC injection) shows higher bioavailability than the aqueous mAb SC injection. See FIG. 11. It has been demonstrated that the in vivo dissolution behavior of the particles ensured that the compositions can clear the injection site at an increased rate as compared to the standard aqueous formulations, as undissolved particles may potentially trigger an immunogenic reaction. In some embodiments, the composition has improved pharmacokinetics (PK) compared to an aqueous composition comprising at least one therapeutic biologic in soluble form. See FIG. 11. The area under the curve (AUC), from time zero to infinity, represents the total drug exposure across time. Peak concentration is a pharmacokinetic measure used to determine drug dosing. The maximum concentration (Cmax) is the highest concentration of a drug in the blood, cerebrospinal fluid, or target organ after a dose is given. Tmax is the time it takes for a drug to reach the maximum concentration (Cmax) after administration of a drug that needs to be absorbed (e.g. an oral drug). Tmax is governed by the rate of drug absorption and the rate of drug elimination. At Tmax, these are equal. In certain embodiments, the composition has improved AUC, Cmax and/or Tmax compared to an aqueous composition comprising at least one therapeutic biologic in soluble form.

In some embodiments, the administration of hyaluronidase allows increased volume and fluid dispersion of the composition with flocculation agent administered by subcutaneous syringe injection. As disclosed herein, the injection volume of the compositions that were subcutaneously administered without resuspension can be greatly increased by pre-injection with hyaluronidase. As shown in Example 9, these hyaluronidase pre-injection sites showed improved rates of injection of the composition as determined by the appearance of a skin bleb formed at the site of administration at the time of the injection.

Particular embodiments of this disclosure are described herein. Of course, variations, changes, modifications and substitution of equivalents of those particular embodiments will become apparent to those of ordinary skill in the art upon reading the foregoing description. The inventors expect skilled artisans to employ such variations, changes, modifications and substitution of equivalents as appropriate, and the inventors intend for the disclosure to be practiced otherwise than specifically described herein. Those skilled in the art will readily recognize a variety of non-critical parameters that could be changed, altered or modified to yield essentially similar results. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

The disclosure generically described, will be more readily understood by reference to the following examples, which are included merely for purposes of illustration of certain aspects and embodiments of the present disclosure, and are not intended to be limiting.

### Kits

In various embodiments, a kit is envisioned comprising a pharmaceutically effective composition comprising a plurality of therapeutic particles with a flocculation agent suspended in a pharmaceutically acceptable liquid carrier. For example, a kit may include one or more additional containers, each with one or more of various materials (such as reagents, optionally in concentrated form, and/or devices) desirable from a commercial and user standpoint for use of a composition described herein. Non-limiting examples of such materials include, but not limited to, diluents, filters, needles, syringes, cartridges, devices, carrier, package, container, vial and/or tube labels listing contents and/or instructions for use, and package inserts with instructions for use. In some embodiments, the present embodiments contemplate a kit for administering a therapy of the embodiments. The kit may comprise one or more sealed prefilled syringes, cartridges or a portable drug delivery injection device, containing any of the pharmaceutical compositions of the present disclosure. The kit may include, for example, at least a plurality of particles comprising at least one therapeutic biologic as well as reagents to prepare, formulate, and/or administer the components of the embodiments or perform one or more steps of the disclosed treatment methods. In some embodiments, the kit may also comprise a suitable container, which is a container that will not react with components of the kit, such as an Eppendorf tube, a syringe, a bottle, a tube, or a portable drug delivery injection device. The container may be made from sterilizable materials such as plastic or glass. In particular embodiments, the composition is dispensed from a prefilled syringe or a portable drug delivery injection device. In certain embodiments, the kit comprises a syringe or portable drug delivery injection device; and a composition comprising: a plurality of particles suspended in a pharmaceutically acceptable liquid carrier, wherein the particles (e.g., substantially all of the particles) comprise at least one therapeutic biologic or a salt thereof; and a flocculation agent, wherein the concentration of the flocculation agent in the composition is less than about 50 mg/mL; and wherein the concentration of the therapeutic biologic or salt thereof in the composition is greater than about 250 mg/mL.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure for delivering a pharmaceutically effective amount of a therapeutic biologic.

A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label may be used to indicate that the contents are to be used for a specific therapeutic application. In addition, the label may indicate directions for use of the contents, such as in the methods described herein. In certain embodiments, the pharmaceutical compositions are presented in a pack or dispenser device which contains one or more-unit dosage forms containing a therapeutic biologic provided herein. The pack for example contains metal or plastic foil, such as a blister pack. Or, the pack or dispenser device may be accompanied by instructions for administration. Or, the pack or dispenser may be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing therapeutic particles and a flocculation agent provided herein formulated in a compatible pharmaceutical liquid carrier are prepared, placed in an appropriate container, and labeled for treatment of an indicated condition.

In some cases, a liquid composition as provided herein is formulated in a prefilled injection device, e.g., syringe or portable drug delivery injection device. In some embodiments, the particle suspension, e.g., composition, is formulated in a volume to be administered of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or about 2.5 mL. In some embodiments, the volume of the composition to be administered is less than about 2.5 mL. In certain embodiments, the volume of the composition to be administered is less than about 2.0 mL, preferably less than about 1.5 mL, more preferably less than about 1.0 mL, and most preferably less than about 0.5 mL.

In other cases, the method further comprises administering at least one hyaluronan degrading agent, e.g., a pharmaceutically effective amount of at least one hyaluronan degrading agent that is administered simultaneously, sequentially or intermittently with the composition. In some embodiments, the hyaluronan degrading agent, to be administered is a volume of less than about 2.0 L, e.g., less than about 1.8, 1.5, 1.2, 1.0, 0.8, 0.5, 0.3, 0.1 L, or about 800, 700, 600, 500, 400, 300, 200, 100, 90, 80, 70, 60, 50, 40, 30, 20, 10, 9.0, 8.0, 7.0, 6.0, 5.0, 4.0, 3.0, or about 2.5 mL. In some embodiments, the composition to be administered is less than about 20.0 mL. In certain embodiments, the composition to be administered is less than about 10.0 mL. In some embodiments, the composition to be administered is less than about 5.0 mL.

In some embodiments, such a kit comprises a prefilled injection device, e.g., syringe or portable drug delivery injection device, of the disclosure in a blister pack. The blister pack may itself be sterile on the inside. In some embodiments, prefilled injection device, e.g., syringe or portable drug delivery injection device, according to the disclosure may be placed inside such blister packs prior to undergoing sterilization, for example terminal sterilization.

The disclosure generically described, will be more readily understood by reference to the following examples, which are included merely for purposes of illustration of certain aspects and embodiments of the present disclosure, and are not intended to be limiting.

### EXEMPLIFICATION

### Abbreviations

Å angstrom
aa amino acids
BSA bovine serum albumin
° C. degrees Celsius
cm centimeter
d day
DI deionized
DP dispersed phase
EA ethylacetate
ELISA enzyme-linked immunosorbent assay
EO ethyloleate
eq. equivalent
Et ethyl
eV electron-volts
FDS filtered drug substance
FA flocculation agent
FV flocculation volume
g gram
GC gas chromatography
h hour
HDPE high-density polyethylene
hIgG human IgG
HPLC high performance liquid chromatography
HSA human serum albumin
Hz hertz
ID internal diameter
IV intravenous
K thousand
KF Karl Fischer
kJ kilojoules
kPa kiloPascal
kV kilovolts
L liter
LC-MS liquid chromatograph mass spectrometry
LD laser diffraction
LDS liquid drug substance
Lyo lyophilization
m meta
mAb monoclonal antibody
MALDI-MS matrix-assisted laser desorption ionization mass spectrometry
Me methyl
MHz megahertz
min minute
μg microgram
μL microliter
μm micrometer
μM micromolar
mg milligram
mL milliliter
mm millimeter
mM millimolar
mol mole
mPa·s milliPascal second
mTorr milliTorr
N newton
nBA n-butylacetate
nm nanometer
p para
PBS phosphate-buffered saline
PEG polyethylene glycol
PEGA polyethylene glycol polyacrylamide
PI pressure indicator
ppm parts per million
ps picosecond
PS 80 polysorbate 80
PTFE polytetrafluoroethylene
ref relative centrifugal force
RH relative humidity
RP-HPLC reversed phase-high performance liquid chromatography
rpm revolutions per minute
RT room temperature
s second
SC subcutaneous
see second
SEM scanning electron microscopy
SLPM standard liter per minute
t tertiary
tert tertiary
UHMW ultrahigh molecular weight polyethylene
um micrometer
UOM unit of measure
UTW ultra thin wall
UV ultraviolet
V volts
VIS visible
vol % volume percent
wt % weight percent

### Materials

Human IgG (IRHUGGF-LY, >97%) and bovine IgG (IRBVGGF) were obtained from Innovative Research as a powder or as an aqueous solution. Bovine serum albumin (BSA) and human serum albumin (HSA) were purchased from Sigma-Aldrich. The monoclonal antibodies (mAb) were provided and received as an aqueous solution. A biosimilar of Roche's Rituximab was purchased from a vendor that provided the antibody in an aqueous composition as 10 mg/mL rituximab, 9 mg/mL sodium chloride, 7.35 mg/mL sodium citrate dihydrate, and 0.7 mg/mL polysorbate 80. A biosimilar of Roche's trastuzumab was purchased from Genscript Biotech Corporation that provided the antibody as an aqueous composition. Composition of custom “feed solutions” used for processing particles were produced through modifying the received formulation by desalting followed by concentrating and adding desired excipients or by direct buffer exchange. All excipients used in particle composition have been used in existing approved biologics injections. Concentration columns were procured from Millipore Sigma (Amicon® Ultra 15 mL Filters for Protein Purification and Concentration with a 10-50 kDa cut off) and used where necessary to: (i) reach the desired monoclonal antibody concentration, and (ii) exchange buffer/excipients before particle formation. Zeba desalting columns (THERMO FISHER SCIENTIFIC™ 87773) were also used to remove salt from solutions in certain instances. Typically, the ratio of residual salt to protein in the desalted solutions (wt/wt) was <1% as determined form conductivity measurements and/or elemental analysis. All excipients were purchased from Sigma-Aldrich and used as received.

### Methods

FLOWCAM™: Particle sizing was measured using FLOWCAM™; a dynamic image analysis instrument. Samples were diluted to about lmg/mL in isopropanol and passed through a thin channel. Images of particles were recorded and analyzed according to size and shape (count-weighted).

Image Analysis: Particle diameters and circularity were measured using ImageJ analysis on SEM images. The analysis was performed on, for example, at 500× or 1000× images. The ImageJ Particle Analysis tool was run on the image, identifying objects with a circularity of >0.8 and size >0.5 μm with each object outlines. These outlines were visually inspected for good fit. Any mis-identified particles were manually rejected, and any missed particles were manually included and measured using the ImageJ diameter tool. Select microscopy images were chosen for further analysis on the basis of (i) minimal particle overlapping, (ii) good contrast between the particles and the background, and (iii) a resolution providing for particle occupancies of at least 10 pixels. This allowed for particles to be easily identified and reduced resolution-based error. A binary threshold was applied to separate the particles from background, and a watershed segmentation algorithm was applied to ensure that individual particles were measured separately. The ImageJ tool “Analyze Particles” was then applied on the binary picture with the following parameters: circularity between 0.5 and 1.0; size between 5 and infinity square microns; exclude on edges; fill holes. The outlines of the identified particles were overlaid onto the original image. Particles which were misidentified, such as clusters that were identified as a single particle or particles whose outlines do not match the particle, were then discarded. Missing particles were measured by manually tracing the particle's outline and using ImageJ's Measure tool.

Accelerated Storage Protocol: All samples were transferred to glass-bottom plates and 2R Schott vials for aging (typically 2 mL or 4 mL volume, depending on sample). The glass-bottom plates and 2R Schott vials were sealed with parafilm, placed in an oven at 4° C., 25° C., 40° C., 50°, or 60° C., and visually inspected over the aging period to ensure integrity and stability.

Viscosity Measurements: Unless otherwise noted, suspension viscosity was measured using an AR-G2 rheometer (TA Instruments) and a 25 mm plate at 25° C. Measurements were taken at 1000 s−1, which is below the shear rates experienced in 27-gauge needles. Each measurement was repeated three times (about 60 s intervals between repeats) to assess short-term physical stability of the suspensions. Prior to each measurement calibration standards were recorded to validate instrument settings.

Injectability Measurements: Unless otherwise noted, syringe force (injection force) was measured during 0.1 mL/s ejection of a 1 mL suspension (400 mg/mL particle) using a custom force sensor apparatus and a 1-mL Norm-ject model syringe with a 27-gauge ultra-thin-wall needle (TSK).

Karl Fischer: Testing for moisture content was undertaken using Karl Fischer analysis. Approximately 100 mg of particles was heated to 150° C. in an oven and released water was determined coulometrically.

Skeletal Density: Skeletal density was measured by gas pycnometry. The gas was nitrogen or other compatible gases, and the particle mass was 0.0413 g.

Particle Dissolution: Phosphate-buffered saline (PBS) was added to dry particle samples to produce a final concentration of 50 mg/mL (particle mass/mL of solution, 96-well plate). After a period of time, 10 μL aliquot was removed from the sample vial and the absorbance at 280 nm was measured and recorded. The Ab, e.g., mAb, concentration was plotted against time for all samples.

Salt Content: Salt content was recorded by measuring sodium content using Inductively Coupled Plasma Optical Emission Spectroscopy (ICP-OES). A calibration curve was prepared using a sodium standard (ICPTRACECERT®, 1000 mg/L). Quality control was completed using a diluted standard solution at 100 ppm sodium. A sample of particles (˜15 mg) dissolved in 2 vol % nitric acid (10 mL) was then analyzed, resulting in an intensity lower than the instrument detection limit of ˜0.5 ppm for sodium. This indicated a sodium content of less than 0.034 wt % and a total salt content (assuming sodium citrate and sodium chloride to have been removed equally) of less than 0.1 wt %.

Size Exclusion Chromatography (SEC) Measurements: 20 μL Injections of samples (1 mg/mL) were run at a flow rate of 1 mL/min in SEC buffer (25 mM phosphate, 250 mM NaCl pH 6.8) for 15 minutes on an AGILENT ADVANCEBIO™ SEC (300 mm×2.7 μm, 300 A column). Peak analysis was performed by auto-integrating using the following parameters: slope sensitivity=0.5, peak width=0, height reject=0, area reject=0, shoulders off, area percent reject 0, standard tangent skim mode, advanced baseline correction, 0 for front peak skim height ratio, 0 for tail peak skim height ratio, 0 for peak to valley ratio, and 0 for skim valley ratio. Alternatively, 20 μL injections of samples (1 mg/mL) were run at a flow rate of 1 mL/min in SEC buffer (25 mM phosphate, 250 mM NaCl pH 6.8) for 15 minutes on an AGILENT ADVANCEBIO™ SEC (300 mm×2.7 μm, 300 A column). Peak analysis was performed by auto-integrating using the following parameters: slope sensitivity=0.5, peak width=0, height reject=0, area reject=0, shoulders off, area percent reject 0, standard tangent skim mode, advanced baseline correction, 0 for front peak skim height ratio, 0 for tail peak skim height ratio, 0 for peak to valley ratio, and 0 for skim valley ratio.

Cation Exchange Chromatography (CIEX) Measurements: Charge variant analysis was performed for each sample on days 0, 7 and 30 under accelerated storage conditions, using an AGILENT BIOMAB™ NP5, 4.6×250 mm, PEEK ion exchange column. Samples were prepared at 1 mg/mL concentration after overnight dialysis in water. Buffer A was prepared with: 30 mM phosphate, pH: 6.3, and NaCl: 0 mM. Buffer B was prepared with: Buffer A: 30 mM phosphate, pH 6.3 plus NaCl: 175 mM. The samples were run in a gradient starting with 100% Buffer A, ramping up to a 100% Buffer B over a course of 20 min, after which the gradient was set to return to 100% Buffer A and 0% Buffer B in the next 1 min. The system re-equilibrated in 100% Buffer A for 10 min before the injection of the next sample. Integration was performed as a manual skim peak mode to reflect the AGILENT™ data.

Flow Cytometry: 1 Million Raji cells (100 μL per well) were plated per well in a 96 well ‘V-bottom’ plate and 10 μL of mAb, Label, particle, or suspension at a starting concentration of 200 μL was added to the wells. The dilution factor for the mAb label, particle and suspension was 3×. The plate was incubated at 4° C. for 30 min. The plate was centrifuged at 2000 rpm for 5 min and was washed 3 times with PBS. 100 μL of PE-conjugated goat anti-human IgG was added as the secondary antibody at a 1:200 dilution. The plate was centrifuged at 2000 rpm for 5 min and was washed 3 times with PBS. The cells were then resuspended in 200 μL of cold PBS for analysis on a Life Technologies ATTUNE™ NXT flow cytometer.

Scanning Electron Microscopy (SEM): Electron micrographs were collected for select samples with either a HITACHI™ TM3030Plus or a TM1000 tabletop microscope. The samples were immobilized on conductive tape and examined in a low-vacuum anti-charging environment, obviating the need for sample preparation.

Image Analysis: Select microscopy images were chosen for further analysis on the basis of (i) minimal particle overlapping, (ii) good contrast between the particles and the background, and (iii) a resolution providing for particle occupancies of at least 10 pixels. This allowed for particles to be easily identified and reduced resolution-based error. A binary threshold was applied to separate the particles from background, and a watershed segmentation algorithm was applied to ensure that individual particles were measured separately. The ImageJ tool “Analyze Particles” was then applied on the binary picture with the following parameters: circularity between 0.5 and 1.0; size between 5 and infinity square microns; exclude on edges; fill holes. The outlines of the identified particles were overlaid onto the original image. Particles which were misidentified, such as clusters that were identified as a single particle or particles whose outlines do not match the particle, were then discarded. Missing particles were measured by manually tracing the particle's outline and using ImageJ's Measure tool.

Density Analysis: The skeletal density of particles from select samples was determined by examining approximately 0.1 g of powder with an ACCUPYC™ 111340 gas displacement pycnometry system.

Water Content Analysis: The moisture in particles from select samples was determined by placing approximately 0.1 g of powder in an oven with a Karl Fischer titrator and heating the sample.

ELISA Assay: ELISA assay was used on select samples to detect human antibody in a denaturation sensitive manner. Human IgG was first plated in PBS for 1 hour, followed by washing with wash buffer (PBS+0.05% Tween20) three times for 4 minutes, followed by blocking with 2% BSA (Sigma) in wash buffer for 45 minutes, followed by incubation with dilute (20 μg/mL) protein A-HRP (ABCAM™) for 45 minutes, followed by wash buffer three times for 3 minutes, followed by incubation with TMB (ABCAM™) for 10 minutes, finally followed by quenching of the reaction with STOP solution (ABCAM™). The colorimetric readout was conducted on a THERMO MULTISKAN SPECTRUM™.

Subvisible Particle (SvP) Analysis: Subvisible particles (SvPs) were analyzed with a Fluid Imaging Technologies FLOWCAM™ PV-100 system. Samples for analysis were reconstituted in sterile centrifuge tubes with filtered water (MILLI-Q™) to the concentration of interest. Three sets of samples were investigated thereafter. These included (i) a sample of the diluent used for reconstitution, (ii) an aliquot of the feed solution used for the particle formation process, i.e., a sample of the first aqueous liquid, and (iii) the reconstituted material.

Accelerated Storage: Unless otherwise noted, storage was carried out under accelerated conditions for select samples by maintaining them at an elevated temperature (40° C.) for defined periods of time in an incubator or oven. Samples were kept in 2 mL or 4 mL WHEATON™ glass vials and sealed with paraffin film.

Inverse Gas Chromatography (IGC): Powdered samples were analyzed using inverse gas chromatography. Cylindrical columns were packed with 200 to 300 mg of powdered samples to make up a stationary phase. Following an inert gas purge, a series of gas probes was injected on the column. Determination of the retention volume for each probe enabled evaluation of the dispersive and polar components of the surface energy for each sample.

X-Ray Diffraction (XRD): Samples were packed into 0.7 mm diameter glass capillaries. The powder patterns were measured on a PANALYTICAL EMPYREAN™ diffractometer equipped with an incident-beam focusing mirror and an X'CELERATOR™ detector. The patterns (1-50° 20, 0.0167113° steps, 4 see/step, ¼° divergence slit, 0.02 radian Soller slits) were measured using Mo Ká radiation. If static electricity effects (for the case of evaluating a lyophilization control this occurred after grinding in a mortar and pestle) prevented packing the sample into a capillary, its powder pattern was measured from a flat plate specimen on a BRUKER™ D2 Phase diffractometer equipped with a LYNXEYE™ position-sensitive detector. The pattern was measured using Cu Kα radiation from 5-100° 20 in 0.0202144° steps, counting for 1.0 see/step. The standard instrument settings (30 kV, 10 mA, 0.6 mm divergence slit, 2.5° Soller slits, and 3 mm scatter screen height) were employed.

Microflow Particle Sizing (MPS): Flow imaging microscopy for particle size analysis was carried out using a FLOWCAM™ PV-100. To investigate size and dispersity of particles, 5 mg of powder were dispersed in 10 mL of dry isopropanol via sonication. The isopropanol continuous phase prevented the particles from dissolving, i.e., prevented reconstitution. 0.3 mL was injected into the cell and images of the particles were taken using a flow rate of 0.15 mL/minute. Particles with a circularity greater than 0.9 were reported in the analysis and any double images were removed from the analysis, to give a size distribution and dispersity of particles in the range from 1 to 100 μm.

Dynamic Vapor Sorption (DVS): Powders were analyzed using dynamic water vapor sorption. Approximately 50 mg of powdered sample was loaded into the pan of the instrument's microbalance. The sample was held isothermally at 22° C. and the sample mass was monitored throughout the measurement. Following a 0% RH purge to remove surface water, the relative humidity (RH) in the sample chamber was ramped at a constant rate of 4% RH per hour up to 90% RH. The sample was held at 90% RH for one hour, then the RH was reduced to 0% as a step change. The sample was held at 0% RH for one hour, after which the measurement was terminated.

Dynamic Scanning Calorimetry (DSC): Powdered samples were analyzed using dynamic scanning calorimetry. Masses of 5 to 10 mg of powdered samples were loaded into aluminum crucibles and sealed hermetically. Crucibles were loaded into the instrument, and the heat flow into the samples was monitored while the temperature was ramped from −80-200° C., optionally, from 20-180° C., at a constant rate of 5° C./minute.

USP <790>: According to the USP <790> standard, samples of dissolved particles were visually observed against a white and black background under lighting conditions greater than 2000 lux. Matte-finished high-density polyethylene sheets were selected for the background to reduce glare. The illuminance at the viewing point was confirmed with a lux meter (Dr. Meter, LX1330B). The samples were swirled before being held up to the backgrounds and viewed for 5 sec.

Flocculation volume: Flocculation volume can be measured by suspending the particles in the desired liquid carrier at a suitable concentration. The suspension is then agitated, typically by shaking or mechanical means, to ensure a uniform dispersion of particles in the fluid at the start of measurement. Immediately after agitation ceases, timing begins. The mixture is left to stand and the degree of settling or sedimentation of the solid is observed and recorded over time.

### Example 1

General protocol for the preparation of protein particles: 1. An aqueous protein solution was adjusted to reach a final concentration of 50-160 mg/mL. 2. Excipients were then added to the resulting solution and then filtered through a small 0.22 μm filter to remove any extrinsic solids from the resulting aqueous “feed” solution. 3. A dehydration solvent (e.g., n-butyl acetate) was added to a vessel (2 mL to 200 L vessel). 4. The protein feed solution was then added to the vessel at various flow rates (e.g., up to 100 mL/min). 5. The mixture was then stirred (e.g., subject to shear field or up to 25,000 rpm) for a period of time (e.g., up to 5 minutes) to produce aqueous droplets, which subsequently were dehydrated (time dependent on the dehydration solvent volume, rpm of the mixture and flow rate) to form the protein particles suspended in the mixture. 6. The protein particles were then separated from the liquid mixture (e.g., filtration, centrifugation and decanting, etc.). 7. The liquid mixture (aqueous liquid and dehydration solvent) was removed. 8. The residual solvent (e.g., aqueous liquid and dehydration solvent) in the protein particles was removed from the protein particles (e.g., vacuum drying, gas drying, gas sparging, extraction solvent, etc.) and the moisture content in the particles was then adjusted (e.g., humid gas, extraction solvent, etc.). 9. A portion of the protein particles were then dissolved in DI water and incubated for complete redissolution for characterization. 10. The solution was then analyzed to measure protein quality (SvP analysis using FLOWCAM™ and soluble aggregate (e.g., SEC), fragmentation (e.g., SEC), change in charge variant (e.g., ion exchange chromatography (SCEX)) analysis using HPLC-SEC) and protein concentration. Protein concentrations in the range of 200 mg/mL to 800 mg/mL have been processed according to the general protocol (particle protein loading of at least 60 to 93% (w/w)). Particles with up to 85% protein loading showed improved stability compared to the starting aqueous feed solution.

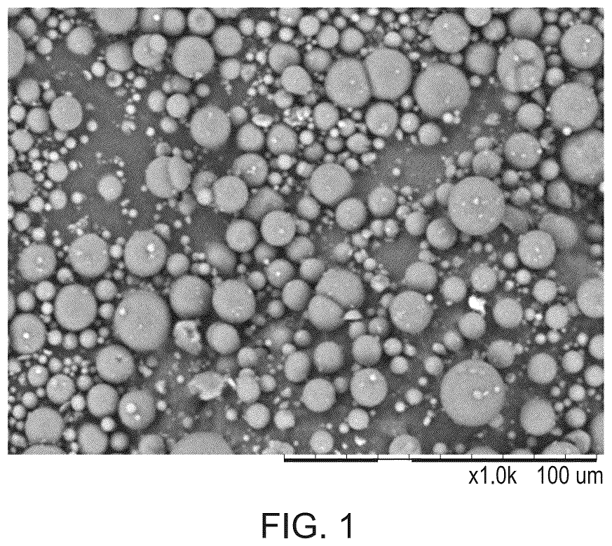

Preparation of Ab particles in the target size and dispersity ranges: Rituximab, trastuzumab, human or bovine IgG particles were formed under general protocol and 10 mg of particles were provided for analysis. The average particle size, D10, D50 and D90 were reported based on particle size analysis (FLOWCAM™), count-weighted. The average particle size of the particles, D10, D50 and D90 was determined to have an average particle size between 5-50 μm and D90<60 μm. The particles had the following particle size statistic: mean diameter=5-50 μm. SEM of the particles for the batches were obtained. Images were taken at 1000× magnification as shown in FIG. 1. The image processing and statistics were performed using ImageJ analysis, count-weighted and provided protein particles with desirable physical characteristics (e.g., circularity, internal void spaces, protein by weight, stability and quality, etc.) that were used for the suspension studies.

Determination of aggregation, fragmentation and change in charge variants in particles by SEC: Dry powder samples were dissolved to 5% (w/v) in ultrapure water and shaken at 60 RPM. After determining triplicate protein concentrations, the samples were diluted to 1 mg/mL with PBS and syringe filtered into HPLC vials. Samples in carrier liquid were resuspended and each sample was immediately centrifuged at 500 RPM, with the resulting carrier liquid supernatant being aspirated (and discarded) without disturbing the sedimented particles. After adding water to the particles and mixing, the samples were shaken at 60 RPM. Following an initial protein concentration determination (from the bottom or aqueous layer), each sample was diluted with PBS and the protein concentration was measured in triplicate (from the bottom or aqueous layer). Each sample was then diluted with PBS and syringe filtered into HPLC vials, with the needle being discarded before filtration (to prevent transfer of carrier liquid). All samples were analyzed using an AGILENT™ 1260 Infinity II Bio-inert LC System and TSKgel SuperSW HTP column (4 μm, 4.6 mmID×150 mmL) column equilibrated with 200 mM Arginine-HCl, 100 mM Sodium Phosphate pH 6.5. The autosampler and column compartment were maintained at 4° C. and 20° C., respectively, and UV absorbance was monitored at 280 nm. The run time was 10 minutes. The % areas of the integrated high molecular weight (aggregates), monomer and low molecular weight peaks were reported.

Determination of aggregation, fragmentation and change in charge variants in particles by SCEX: Dry powder samples were dissolved to 5% (w/v) in ultrapure water and shaken at 60 RPM. After determining triplicate protein concentrations, the samples were diluted with ultrapure water and syringe filtered into HPLC vials. Samples in carrier liquid were resuspended and each sample was immediately centrifuged at 500 RPM, with the resulting carrier liquid supernatant being aspirated (and discarded) without disturbing the sedimented particles. After adding water to the particles and mixing, the samples were shaken at 60 RPM. Following an initial protein concentration determination (from the bottom or aqueous layer), each sample was diluted with ultrapure water and the protein concentration was measured in triplicate (from the bottom or aqueous layer). Each sample was then diluted with ultrapure water and syringe filtered into HPLC vials, with the needle being discarded before filtration (to prevent transfer of carrier liquid). All samples were analyzed using an AGILENT™ 1260 Infinity II Bio-inert LC System and THERMO SCIENTIFIC MABPAC™ SCX-10 RS column (5 μm, 2.1 mmID×150 mmL) equilibrated with 100% mobile phase A (THERMO SCIENTIFIC™ 1× CX-1 pH Gradient Buffer A). The gradient is 0-20% mobile phase B (THERMO SCIENTIFIC™ 1× CX-1 pH Gradient Buffer B). The autosampler and column compartment were maintained at 4° C. and 30° C., respectively, and UV absorbance was monitored at 280 nm. The % areas of the integrated acidic, neutral (main) and basic peaks were reported.

### Example 2

General protocol for protein particle compositions: Rituximab, trastuzumab or IgG (human or bovine) particles containing excipients (e.g., histidine, arginine·HCl, PS80, PS20, trehalose, NaCl, sucrose, methionine, proline, sodium phosphate), were made into a suspension at 200-700 mg/mL concentration in an organic carrier liquid or mixture of at least two organic carrier liquids (e.g., ethyl oleate, sesame oil, medium-chain triglycerides (MCTs), propylene glycol diesters of saturated plant fatty acids C8 and C10 (PGD), triacetin, caprylic triglyceride). This was achieved by adding protein particles to the organic carrier liquid and using a mixer (e.g., rotor stator, etc.) to generate a dilute suspension. The dilute suspension was sieved through a 60 μm filter and the suspension was adjusted to the desired protein concentration. To this suspension, a flocculation agent was added to reach a total concentration of flocculation agent up to 10 mg/mL with a protein concentration of 200-800 mg/mL. This suspension was initially mixed using vortexed and/or mechanically mixed for 1 minute to create the desired flocculation volume which remained stable for at least 1 month.

HIgG particle compositions: HIgG particles (74% protein: 2% histidine: 24% arginine: 1% PS80 (or PS20) or 78% protein: 2% histidine: 19% arginine: 1% PS80 or 89% protein: 3% histidine: 7% arginine: 1% PS80 or 89% protein: 3% histidine: 7% arginine: 1% trehalose (or methionine) or 88% protein: 3% histidine: 7% arginine: 1% PS80:1% methionine (or NaCl, sucrose, proline, sodium phosphate)) were made into a suspension at 500 mg/mL concentration in ethyl oleate. This was achieved by adding 19-23 g of particles to about 450 mL of ethyl oleate, and using a rotor stator mixer to generate a dilute suspension. The dilute suspension was sieved through a 60 μm filter and adjusted to a concentration of 500 mg/mL of HIgG by centrifugation. To an aliquot of this suspension, a flocculation agent (polysorbate 80) was added with a pipette to reach a final total concentration of up to 10 mg/mL with a HIgG concentration of 500 mg/mL. This suspension was vortexed and then mechanically mixed for 1 minute creating a stable suspension (flocculation volume) that remained consistent for at least one month compared to the particle suspension without the flocculation agent. HIgG and BIgG were used interchangeably. Characterization of the protein was accomplished according to Example 1 and showed improved stability compared to the starting aqueous feed solution.

Rituximab particle compositions: Rituximab particles (98% protein: 2% histidine or 80% protein: 2% histidine: 18% arginine) were made into a suspension at 500 and 700 mg/mL concentration in ethyl oleate. This was achieved by adding particles to about 450 mL of ethyl oleate, and using a rotor stator mixer to generate a dilute suspension. The dilute suspension was sieved through a 60 μm filter and adjusted to a concentration of 500 or 700 mg/mL of rituximab by centrifugation. To an aliquot of this suspension, a flocculation agent (polysorbate 80) was added with a pipette to reach a final total concentration of up to 10 mg/mL with a rituximab concentration of 500 or 700 mg/mL. This suspension was vortexed and then mechanically mixed for 1 minute creating a stable suspension (flocculation volume) that remained consistent for at least one month compared to the particle suspension without the flocculation agent. Characterization of the protein was accomplished according to Example 1 and showed improved stability compared to the starting aqueous feed solution.

Trastuzumab particle compositions: Trastuzumab particles (71% protein: 2% histidine: 26% arginine: 1% PS80) were made into a suspension at 500 or 700 mg/mL concentration in ethyl oleate according to the rituximab protocol. A stable suspension (flocculation volume) that remained consistent for at least one month was produced according to the rituximab protocol. Characterization was accomplished according to Example 1 and showed improved stability compared to the starting aqueous feed solution.

### Example 3

Protein particle compositions with high concentration, stable flocculation volume, and low injection force:

The units “mPa·s” and “cP” are used herein interchangeably in the broadest sense.

Rheology: A suspension of rituximab, trastuzumab, bovine IgG, or human IgG particles was created with the following characteristics: protein loading >250 mg/mL and apparent viscosity <20 cP (mPa·s). The viscosity was measured using a parallel plate rheometer or other methods that are known in the art. Suspensions were prepared in carrier liquids at various concentrations. At concentrations >300 mg/mL viscosities around 20 cP (mPa·s) were measured. When lower viscosity carriers are considered, even higher concentrations can be achieved.

Rheology (days 0, 7, 30 at 40° C.): The viscosity of the suspension of particles (rituximab, trastuzumab, bovine IgG, or human IgG) over time was tracked to ensure that it does not vary by more than 5% over 7- and 30-day accelerated storage. At each time point, the suspension had a viscosity <20 cP (mPa·s) with a protein loading >250 mg/mL. The concentration condition was met based on the preparation of a 280 mg/mL protein suspension. The Day 0 sample was measured immediately. Day 7 and Day 30 samples were aged according to the aging procedure outlined above. Measurements were taken at shear rates of 1000 s⁴. At Day 0, the viscosity of the suspension was measured at 16.87 cP. On Day 7, the viscosity reduced to 16.33 cP. At Day 30, the viscosity increased 29% as compared to Day 0 to 21.83 cP, and is slightly above the 20 cP limit. However, using such a small plate (25 mm), small instrument gap (150 microns), and small sample volume, it has been estimated that error in the measurement could be as high as 30%.

Karl Fischer Titration: The water content of the solid particles (rituximab, trastuzumab, bovine IgG, or human IgG) was determined to define the component make-up of the particles using Karl Fischer titration. The water content of the particles was measured, recorded and determined to be 1-6 wt % for each of rituximab, trastuzumab, bovine IgG, or human IgG.

Determination of viscosity: A 25 mm 3-degree cone (CP25-3, Anton Paar) is loaded onto the rheometer (Anton Paar Modular Compact Rheometer (MCR) Series 92). The plate was warmed up to 25° C. The suspension with a flocculation agent was vortexed until visually homogenous. 250 uL is pipetted onto the base plate and the samples were measured at a shear rate of 950 1/s.

Determination of protein content: Particle suspensions with a flocculation agent were dispersed by vortexing, diluted to 2.5% (v/v) in ultrapure water and shaken at 60 RPM for 30 minutes; corresponding dry powder samples were dissolved to 5% (w/v) in ultrapure water and shaken at 60 RPM for 30 minutes. After determining triplicate protein concentrations (from the bottom or aqueous layer for suspensions), the percent mass of protein in particles was calculated by dividing the product of the averaged redissolved protein particle concentration and redissolution volume by the mass of microparticles. The averaged concentration of suspensions (corrected by aqueous/formulation carrier liquid dilution factor) was then divided by this percent mass of protein in particles to calculate the theoretical mass of particles in suspension. The results are comparable to the protein content shown in Examples 1 and 2.

Dissolution: The dissolution rates were recorded for each suspension on days 0, 7 and 30. Dissolution was recorded at various time points which confirmed dissolution of the particles within ˜20 minutes and ˜60-80 minutes for the suspension. The particles and suspension were dissolved in PBS to a final concentration of 10 mg/mL and rocked on a rocker. Dissolution of particles on days 0, 7 and 30 were also stored at 40° C.

USP<790>: The presence of visible particles was determined in dissolved samples of particles. USP <790> was used to determine if there were particles present in the “visible” range (>100 μm). Observations of dissolved particles were used to assess the presence of visible particles. The observations were made briefly (5 seconds) with a white and black background under the appropriate lighting. Some potential small particles were observed, but were difficult to see by eye likely classifying them as subvisible particles. Given that these particles may be considered subvisible, other methods have been used to further investigate as described below.

Subvisible Particle Analysis: The subvisible particle matter present in dissolved particles was investigated. Sub-visible particle analysis was performed after dissolution of the particles. This data was compared to other competitive particle formation technologies. The SvP analysis was performed by a particle analyzer where the particle count adjusted for background signal in the control sample. The subvisible particle counts measured in the samples were lower than any other completing technology including standard lyophilization.

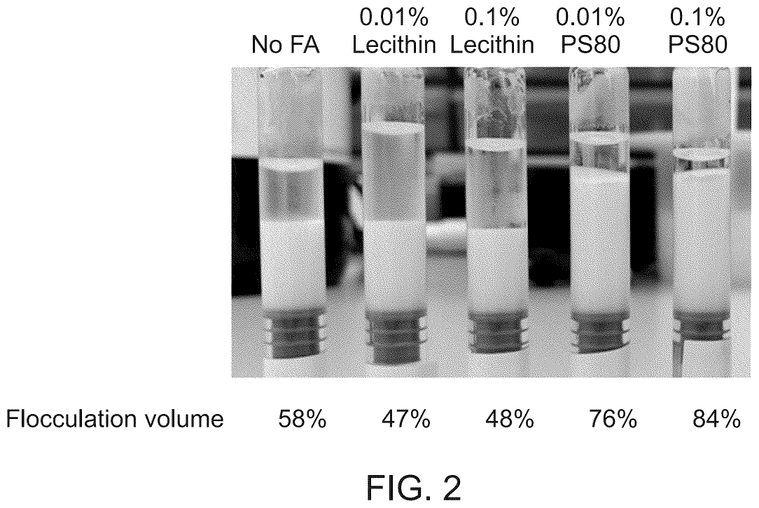

Flocculation Volume: BIgG particles were added to 3 mL of ethyl oleate followed by the addition of a flocculation agent (lecithin or PS 80) at 0.1% to 0.01% by weight to form a protein concentration of 500 mg/mL. The suspension was initially mixed and the flocculation volume was calculated and compared to a suspension without a flocculation agent. FIG. 2 shows the effect of a flocculation agent on particle agglomeration and flocculation volume. Lecithin at 0.01% and 0.1% by weight reduces particle agglomeration and flocculation volume. PS 80 0.01% and 0.1% by weight increases particle agglomeration and flocculation volume compared to a suspension without a flocculation agent. The reduction of the flocculation volume was not observed and remained stable for at least three months at 25C in both cases.

Injectability: The force of injecting the suspension with a flocculation agent through a needle was measured and verified that the injection force remained the same after 24 hours without resuspension prior to injection. As obtained above, a suspension of BIgG particles at 500 mg/mL concentration of protein and PS80 at 0.1% by weight in EO was loaded into a 5 mL syringe with an 18-G needle (3 inches). The injection force was less than 2.0 N and remained the same after 24 hours of storage. For reference, the force of actuating an empty syringe equipped with a syringe needle using a 15 mPa·s standard was compared and the results showed that the injection forces were all below 2.0 N. These studies show that the force of injecting the composition remained stable for at least one month.

### Example 4

Particle and suspension quality studies:

Design: A procedure similar to Examples 1-3 was employed to obtain a suspension of particles with flocculation agent. The stability of the protein particle suspensions with flocculation agent was assessed over the course of three months at 5, 25, and 40° C. to determine the stability of the particle suspension composition in comparison to the protein liquid drug substance (LDS), e.g., starting aqueous feed solution.

Composition: A procedure similar to Examples 1-3 was employed to obtain a suspension of particles with flocculation agents (600 mg/mL protein and 0.1 mg/mL PS80; 74% protein: 2% histidine: 24% arginine: 1% PS80) formulated into particles which were then suspended in ethyl oleate.

Buffer Exchange by Tangential Flow Filtration (TFF): Protein feed solutions were prepared by diafiltration into the required buffer cocktails. A KrosFlow KR2i TFF system (Repligen) equipped with a hollow fiber filter module (MiniKros Sampler) was used to perform the feed preparation. A number of diafiltration volume exchanges were performed with the appropriate buffer for each formulation.

FIB-SEM Imaging: The particles were imaged using a scanning electron microscope (HITACHI™, TM-1000). A sample of the particles was mounted onto an adhesive stage for analysis. Images were captured at varying magnifications using an accelerating voltage of 15 kV.

Buffer Exchange by Tangential Flow Filtration (TFFR: Protein feed solutions were prepared by diafiltration into the required buffer cocktails. A KrosFlow KR2i TFF system (Repligen) equipped with a hollow fiber filter module (MiniKros Sampler) was used to perform the feed preparation. A number of diafiltration volume exchanges were performed with the appropriate buffer for each formulation.

SEM Imaging: The particles were imaged using a scanning electron microscope (HITACHI™, TM-1000). A sample of the particles was mounted onto an adhesive stage for analysis. Images were captured at varying magnifications using an accelerating voltage of 15 kV.

Karl Fischer Coulometry: Testing for moisture content was performed by Karl Fischer analysis using a MetroOhm (899 coulometer) equipped with an 860 KF THERMOPREP™ oven. Particles were heated to 165° C. in an oven and the released water was determined coulometrically.

Particle Dissolution: Water was added to dry particle samples to produce a final protein concentration. Samples are placed on a nutating mixer at 60 RPM for a period of time. The terminal dissolution concentration was recorded by removing an aliquot from the sample and measuring the absorbance at 280 nm (using the extinction coefficient E1%=1.69 L·g−1·cm−1).

Turbidity: An aliquot of reconstituted particle solution was transferred to a 1-cm path length cuvette. The absorbance at 405 nm was recorded using a NANODROP™ One UV-VIS spectrophotometer (THERMO SCIENTIFIC™). The results showed improved turbidity compared to the starting aqueous feed solution and particles.

Size-Exclusion Chromatography (SEC) Measurements: Injections of samples were run at a flow rate of about 0.35 mL/min using a mobile phase comprised of 100 mM sodium phosphate monobasic and 200 mM L-arginine monohydrochloride, pH 6.5 for 10 minutes on a Tosoh TSKgel SuperSW mAb HTP (4.6 mm ID×15 cm L) column. Peaks were manually inspected to ensure accurate identification and analysis was performed by autointegration using parameters known in the art.

Strong Cation-Exchange Chromatography (SCEX): Injection of samples dissolved in MILLI-Q™ water were run at a flow rate of 0.4 mL/min using a gradient method that starts at 100% mobile phase pH gradient buffer A to 100% mobile phase pH gradient buffer B followed by washing and re-equilibration for a total run time of 40 minutes on a MAbPac™ SCX-10 RS Analysis Column, 2.1 mm ID×15 cm L, 5 μm column. Peaks were manually inspected to ensure accurate identification and analysis was performed by autointegration using parameters known in the art.

Hydrophobic Interaction Chromatography (HIC): Injection of samples dissolved in diluent comprised of 750 mM Ammonium Sulfate, 50 mM Sodium Phosphate Monobasic Dihydrate, pH 6.0 (1 mg/mL) were run at a flow rate of 1 mL/min using a gradient method that starts at 50% mobile phase A comprised of 2M ammonium sulfate, 50 mM sodium phosphate monobasic dihydrate, pH 6.0 to 100% mobile phase B comprised of 50 mM sodium phosphate monobasic dihydrate, pH 6.0 followed by washing and re-equilibration using 50% mobile phase A for a total run time of 60 minutes on a MAbPac™ HIC-20 HPLC Column, 5 μm, 4.6 mm ID×25 cm L column. Peaks were manually inspected to ensure accurate identification and analysis was performed by autointegration using parameters known in the art.

Particle Sizing (Laser Diffraction): Particle size analysis was conducted via laser diffraction using a Horiba LA-960S. Dry particles were suspended in isopropyl alcohol at a concentration of approximately 0.1 mg/mL. The Particle suspension was sonicated within the particle measurement instrument to ensure homogeneity and then circulated and agitated by the Horiba particle size analyzer. Particle size analysis was conducted using a mobile phase of isopropyl alcohol and the volume average particle size distribution was calculated.

Microflow Imaging: Flow imaging microscopy (FLOWCAM™, Fluid Imaging Technologies) was performed to quantify subvisible particulates in protein LDS and the particle formulation. Particles were first redissolved using the particle dissolution method described above and diluted to 1 mg/mL with ultrapure water. For analysis, the aqueous sample was introduced at a flow rate of 0.15 mL/min. The resulting particle counts are recorded and reported per mg of protein.

Results: The stability of dry particles and particle suspensions with flocculation agent were evaluated alongside the protein liquid drug substance (LDS). Stability was tracked at 5, 25, and 40° C. with data collected at 7, 14 days, 1, 3, 6, 9 and 12 months. Particle stability, protein stability of the LDS, and suspension were measured at each time and temperature. Analysis of the particles and particle suspensions confirmed that protein quality remained constant as measured by the monomer profile (SEC), maintenance of charge variant profile (CEX), isoforms/presumed oxidation (HIC), and colloidal stability (turbidity and subvisible particles). In each case, the protein feed solution was measured against the particles and particle suspensions with a flocculation agent. Particles stored as dry powder and particle suspension at 5, 25, and 40° C. showed a smooth and spherical morphology. Over the course of three months at these temperatures, no change in the particle morphology or the particle size distribution was observed. The moisture content of the particles remained constant for all storage temperatures and length of time. Analysis of protein aggregates were measured by SEC up to 12 months at 5, 25, and 40° C. For the standard, the effect of excipient selection (histidine, arginine·HCl, and PS80) on the stability of hIgG particles (without flocculation agent') versus Δ (Mon+Dim) % and Δ (Dim+Agg) % was conducted and stored at 40° C. for 12 months to assess aggregation over time when compared to an LDS sample. As shown in FIG. 3, LDS sample stored at 40° C. for 12 months demonstrated high protein aggregation in comparison to the hIgG particle and suspension with a flocculation agent samples stored at the same temperature (40° C. for 12 months).

The low rate of aggregation for the particle suspensions with flocculation agent demonstrated improved stability as compared to protein LDS. The charge variant profile of protein was measured by SCEX up to 90 days at 5, 25, and 40° C. No appreciable change was observed for particles and particle suspensions with flocculation agent which demonstrated improved stability at 40° C. as compared to protein LDS. HIC was used to measure potential oxidation of protein. The degree of presumed global oxidation of protein was measured by HIC up to 90 days at 5, 25, and 40° C. No appreciable change was observed indicating that the particles and particle suspensions with flocculation agent demonstrated improved stability with respect to oxidative stress at 25 and 40° C. as compared to protein LDS at the same temperatures and times. To further probe the protein quality of the redissolved particles in aqueous media, visible and subvisible particulates were analyzed after storage at various temperatures. Upon dissolution, the protein solutions were essentially free of visible particulates and subvisible particulates after storage at 40° C. up to 90 days. Protein concentration, viscosity and injection force did not significantly change at all storage times and temperatures for the particle suspensions with flocculation agent.

### Example 5

Stability of protein with a flocculation agent (days 0, 7, 30 at 40° C.) by structural assays:

Size Exclusion Chromatography (SEC): The objective was to collect SEC data to assess the degradation of the protein monomers in the presence of flocculation agent through storage at 40° C. for 30 days. SEC was performed based on the protein liquid drug substance (LDS), particles and suspensions with flocculation agent. The monomer, aggregate and fragment data for each sample was obtained. The SEC data was recorded and demonstrated good stability of protein monomers through processing and storage with flocculation agent on day 0, 7 and 30 after storage at 40° C. The 30-day sample for the LDS had poorer preservation of the monomers as compared to the particle suspensions with flocculation agent, which each had >98% preservation of monomers.

Cation Exchange Chromatography (CIEX): CIEX data was collected to assess the distribution of charge variant species and their changes through storage in the presence of flocculation agent at 40° C. for 30 days. CIEX was performed on the LDS, particles and suspensions with flocculating agent. The acidic, main, and basic species data for each sample was obtained and analyzed. Select CIEX data points highlighted the acidic shift that was experienced by the label formulation compared to the absence of such a shift in the suspensions after 30 days of aging. The protein from the suspension resembled the unprocessed material whereas the LDS experienced high degradation. The CIEX data was recorded as proposed and demonstrated good stability of charge variants through storage with flocculation agent on day 0, 7 and 30 after storage flocculation agent at 40° C. The 30-day sample for the LDS had a drastically low preservation of the charge variant distribution as compared to the particle suspension with flocculation agent due to an acidic shift in variants.

Aggregation, Fragmentation and Change in Charge Variants of the Protein: Aggregation and fragmentation data were collected using Size Exclusion Chromatography (HPLC-SEC) according to Examples 1-3, unless otherwise noted. Strong Cation Exchange Chromatography (CIEX) was used to collect the distribution of charge variant species and their changes according to Examples 1-3, unless otherwise noted. The results showed improved stability for the suspension with flocculating agent compared to the starting LDS.

### Example 6

Flocculation agent and protein concentration studies:

The average glide force was plotted against particle suspension concentrations of up to 700 mg/mL (74% protein: 2% histidine: 24% arginine: 1% PS80). 20 N (Newtons) was accepted as the reference to typical glide force limits.

Flocculation volume by increasing flocculation agent and protein concentration: Protein particle suspensions using BIgG were created at a range of concentrations from 300 to 700 mg/mL using ethyl oleate as the liquid carrier with flocculation agent (PS80) concentrations from 0 to 10 mg/mL and stored for 24 hours. FIG. 4 shows a list of the flocculation agent and protein concentration parameters for the flocculation volume study. Table 1 shows that as the concentration of BIgG and PS80 increases, the flocculation volume increases.

TABLE 1

| BlgG | PS80 | FV |
|---|---|---|
| (mg/mL) | (mg/mL) | % |
| 400 | 0 | 53 |
| | 0.01 | 54 |
| | 0.1 | 61 |
| | 1 | 72 |
| | 10 | 67 |
| 500 | 0 | 70 |
| | 0.01 | 70 |
| | 0.1 | 76 |
| | 1 | 86 |
| | 10 | 81 |
| 600 | 0 | 79 |
| | 0.01 | 79 |
| | 0.1 | 84 |
| | 1 | 94 |
| | 10 | 90 |
| 700 | 0 | 84 |
| | 0.01 | 82 |
| | 0.1 | 88 |
| | 1 | 93 |
| | 10 | 94 |

Injectability by increasing flocculation agent and protein concentration: Protein particle suspensions using BIgG were created at a range of concentrations from 500 to 700 mg/mL using ethyl oleate as the liquid carrier with flocculation agent (PS80) concentrations from 0 to 10 mg/mL using a 20-gauge and 22-gauge 13 mm (half-inch) needle (Japan Bio Products) attached to a 1 mL syringe. Table 2 shows that as the concentration of BIgG and PS80 increases, the flocculation volume increases with no substantial change in injection force.

TABLE 2

| BlgG | PS80 | FV | Syringe |
|---|---|---|---|
| (mg/mL) | (mg/mL) | % | Force/N |
| 500 | 0.10 | 92 | 2.0 |
| | 1.00 | 86 | 2.0 |
| | 10.0 | 93 | 2.6 |
| 600 | 0.00 | 90 | 2.9 |
| | 0.01 | 89 | 2.7 |
| | 0.10 | 86 | 2.8 |
| | 1.0 | 96 | 2.0 |
| | 10.0 | 96 | 3.0 |
| 700 | 1.00 | 87 | 2.5 |
| | 10.0 | 94 | 2.2 |

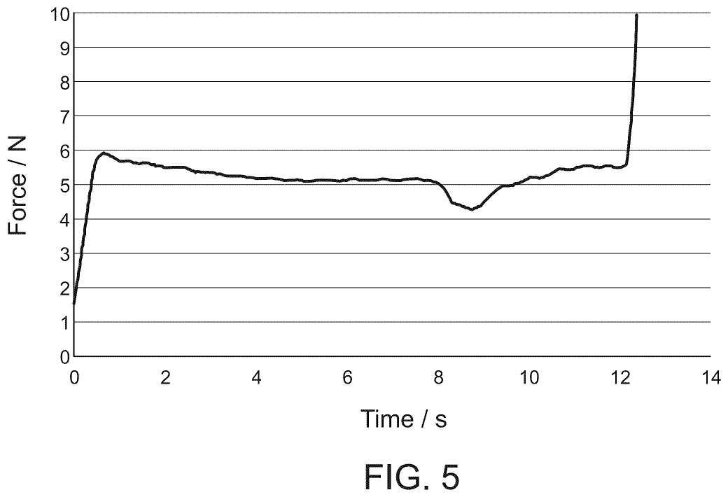

Injectability using caprylic triglyceride: Protein particle suspensions using BIgG were created at a concentration of 620 mg/mL (74% protein: 2% histidine: 24% arginine: 1% PS80) using caprylic triglyceride as the carrier liquid with flocculation agent (PS80) concentration at 0.1 mg/mL. No change in flocculation volume was observed after 24 hours. Using a 22-gauge 13 mm (half-inch) needle (Japan Bio Products) attached to a 1 mL syringe, the injection force was 5.2 N at 0.1 mL/s as shown in FIG. 5.

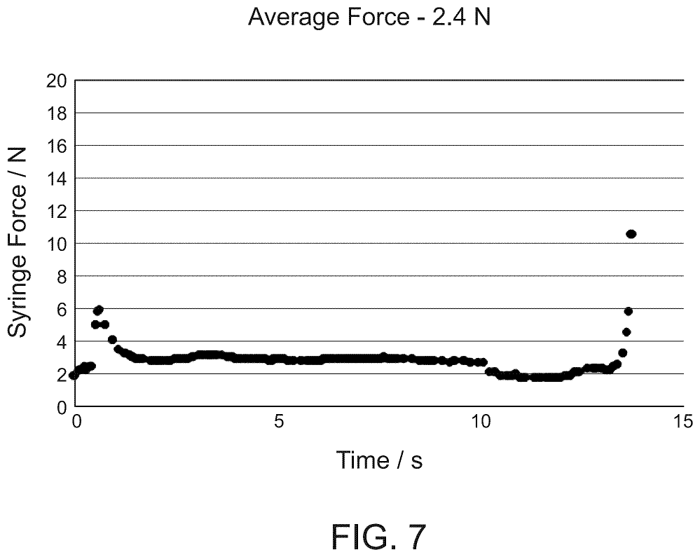

Flocculation volume and storage: Protein particle suspension using BIgG was created at a concentration of 556 mg/mL (74% protein: 2% histidine: 24% arginine: 1% PS80) using ethyl oleate as the carrier liquid with a flocculation agent (PS80) concentration at 0 mg/mL and 10 mg/mL, and stored for 1 month at 4° C. to 25° C. As shown in FIG. 6, no change in flocculation volume was observed at 4° C. or 25° C. after 1 month when PS80 was added to the suspension. Moreover, the injection force remained substantially the same at 2.4 N using a 20-gauge 13 mm (half-inch) needle (Japan Bio Products) attached to a 1 mL syringe (up to 14 seconds). See FIG. 7.

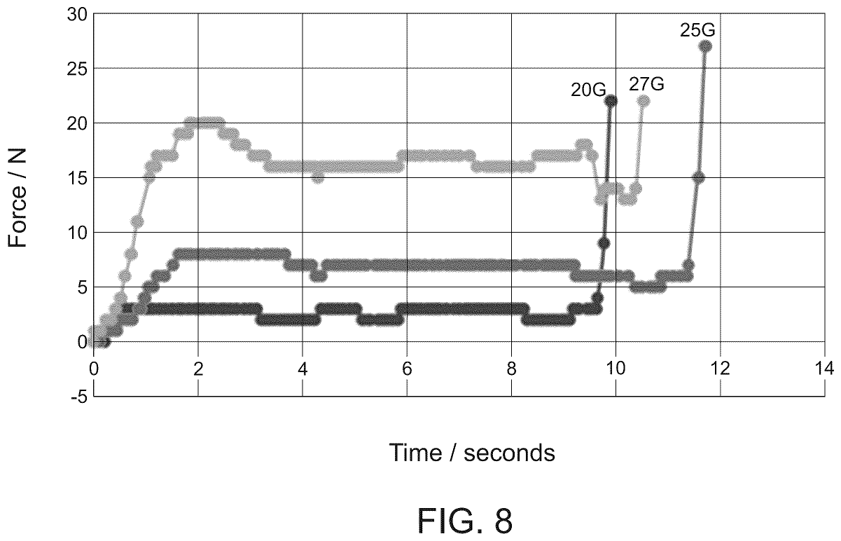

Injection force by needle gauge: Protein particle suspensions using BIgG were created at a concentration of 640 mg/mL (74% protein: 2% histidine: 24% arginine: 1% PS80) using ethyl oleate as the liquid carrier with flocculation agent (PS80) concentration of 10 mg/mL, using a 20-gauge, 25-gauge, and 27-gauge 13 mm (half-inch) needle (Japan Bio Products) attached to a 1 mL syringe. After 24 hours, the flocculation volume remained at 100% with no substantial change in injection force for all three needle sizes (3 N for 20-gauge, 6 N for 25-gauge, and 16 N for 27-gauge). See FIG. 8.

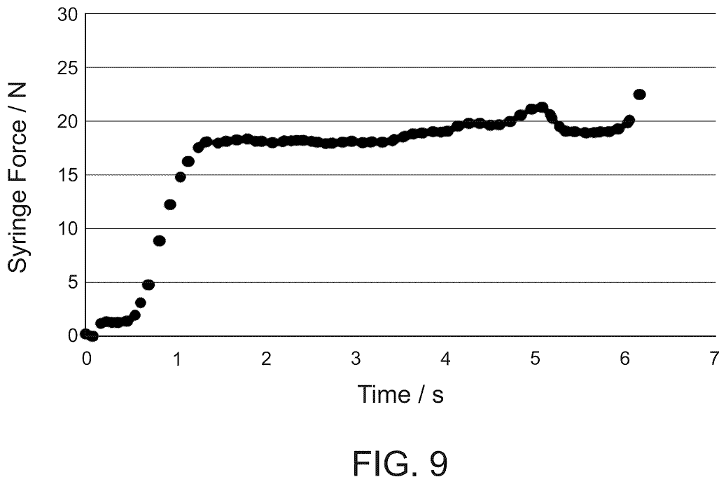

Injection force using 27-gauge UTW needle: Protein particle suspension using BIgG was created at a concentration of 625 mg/mL (74% protein: 2% histidine: 24% arginine: 1% PS80) using ethyl oleate as the liquid carrier with flocculation agent (PS80) concentration of 10 mg/mL, using a 27-gauge UTW, 13 mm (half-inch) needle (Japan Bio Products) attached to a 1 mL syringe. After 24 hours, the flocculation volume remained at 100% with no substantial change in injection force (17.4 N). FIG. 9 shows the injection force in relation to injection time using a 27-gauge UTW needle at a protein concentration of 625 mg/mL.

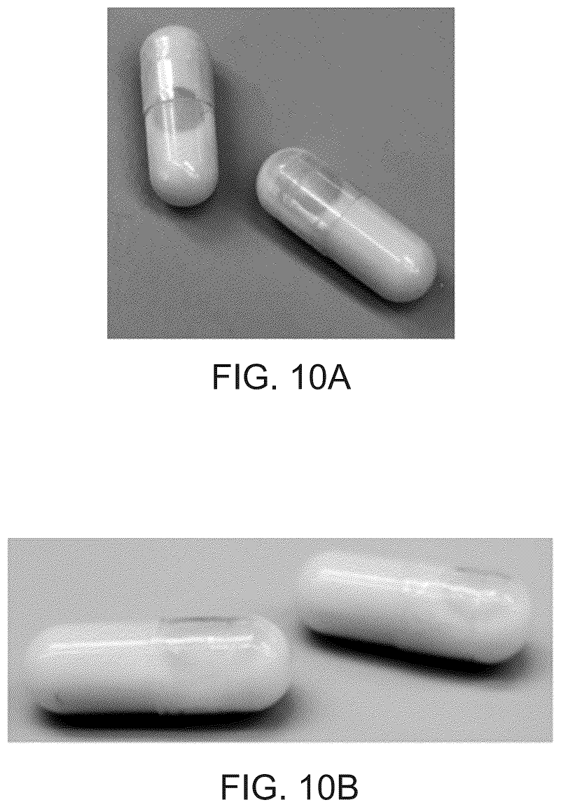

Capsule storage: Protein particle suspension using BIgG was created at a concentration of 556 mg/mL using ethyl oleate as the liquid carrier with flocculation agent (PS80) concentration of 10 mg/mL and added to two gelatin capsules (18 mm long, Medlab) with no air bubbles. After 1 month, the flocculation volume remained at 100% with no observable sedimentation of the protein particles. See FIGS. 10A and 10B.

### Example 7

Pharmacokinetics (PK): The subcutaneous administration of Ab microparticle suspensions show higher bioavailability than the aqueous Ab injection:

Composition: The scope of the study involves four cohorts having the following compositions, 1) IV Ab Aqueous Rituximab: 30 mg/mL 2) SC Ab Aqueous: 30 mg/mL Rituximab (anti CD-20 antibody), 4 mg/mL Histidine, 1 mg/mL Trehalose, 0.7 mg/mL Sodium Chloride and 0.6 mg/mL Polysorbate 80, 3) SC Ab protein microparticle suspension: 30 mg/mL Rituximab (anti CD-20 antibody), 4 mg/mL Histidine, 1 mg/mL Trehalose, 0.7 mg/mL Sodium Chloride and 0.6 mg/mL Polysorbate 80 formulated into the particle composition which were then suspended in Ethyl Oleate (non-Aqueous carrier vehicle) with flocculation agent (PS80) at a concentration of 10 mg/mL. The concentration of the suspension was 30 mg/mL; and, 4) SC Suspension vehicle: Ethyl Oleate (non-Aqueous carrier vehicle).

Animals: Female wild-type Albino Sprague Dawley Rats (Charles River Lab) were housed in the animal facility at the Tufts University Comparative Medicine Services (Tufts CMS). All use of animals described were conducted in compliance with the National Research Council's “Guide for the care and use of laboratory animals” and performed following detailed written protocols that were approved by the Institutional Animal Care and Use Committee (IACUC).

Dosing, study design and analysis: All particle composition injections were accomplished using a 100 μL Hamilton glass syringe with a 27-gauge needle. No protocol was implemented to resuspend the particles in the syringe. All aqueous and non-aqueous vehicle injections were accomplished using a 300 μL insulin syringe with staked-in 30-gauge needle. No protocol was implemented to resuspend the particles in the syringe. Sprague Dawley Rats were used for this study. The following study design was used: 1) IV Ab Aqueous: Dose=10 mg/kg; Volume=100 μL; n=5, 2) SC Ab Aqueous: Dose=10 mg/kg; Volume=100 μL; n=5, 3) SC Ab Microparticle suspension: Dose=10 mg/kg; Volume=100 μL; n=5, 4) SC Suspension vehicle: Volume=100 μL; n=3. Plasma samples were collected at 0 h, 3 h, 6 h, 9 h, 1 d, 2 d, 3 d, 5 d, 6 d, 9 d, 12 d, (14 d, 16 d, 19 d, 21 d and 23 d: These time points were below the limit of detection of the assay utilized). The blood from each rat was collected in EDTA capillary tubes (purple) from tail vein bleeds at all data points. The plasma obtained from the blood was diluted to 10,000× for analysis using ELISA. Plasma samples were assessed using anti-human IgG ELISA assay by third party contract research organization (Ray Biotech: RayBio® Human IgG ELISA Kit). Test article aqueous Rituximab S.C. injection: AUC 5.8 (h*mg/mL), Cmax 34.0 (μg/mL), Tmax 57.6 (h). Test article Rituximab microparticle suspension in ethyl oleate (EO) S.C. injection: AUC 6.3 (h*mg/mL), Cmax 36.7 (μg/mL), Tmax 67.2 (h). As shown in FIG. 11, the Rituximab microparticle suspension shows higher bioavailability than the aqueous Rituximab injection.

Toxicity and local tolerability study and analysis: Injection sites were evaluated pre-injection and post-injection for up to 3 Days for edema (swelling) and erythema (redness). The Draize scoring was utilized to score (0-4 maximum) the sites based on the severity of the response. No unusual finding was observed on any of the 5 animals in the composition group (100 mg/kg, 500 mg/mL) on days 0 (pre-injection), 1 (post-injection), and 7 (post-injection).

Erythema and Eschar Formation: No Erythema—0; Very Slight Erythema (Barely Perceptible)—1; Well Defined Erythema—2; Moderate to Severe Erythema—3; Severe Erythema (Beef Redness) to Eschar Formation Preventing Grading of Erythema—4.

Edema Formation: No Edema—0; Very Slight Edema (Barely Perceptible)—1; Slight Edema (Edges of Area Well Defined by Definite Raising)—2; Moderate Edema (Raised Approximately 1 mm)—3; Severe Edema (Raised More Than 1 Mm and Extending Beyond Area of Exposure)—4.

Draize scoring: The absence or presence of findings was recorded for individual animals on days 0 (pre-injection), 1 (post-injection), and 7 (post-injection). All 5 animals in the group have edema and erythema scores of zero over 3 days post injection, indicating that the composition samples were well tolerated with no toxicity.

Detailed clinical observations: Animals were removed from the cages and observations were made. These include, but were not limited to, evaluation of the skin, fur, eyes, ears, nose, oral cavity, thorax, abdomen, external genitalia, limbs and feet, respiratory and circulatory effects, autonomic effects such as salivation, nervous system effects including tremors, convulsions, reactivity to handling, unusual behavior. No unusual finding was observed on any of the 5 animals in the composition group (100 mg/kg, 500 mg/mL) on days 0 (pre-injection), 1 (post-injection), and 7 (post-injection) indicating that the composition samples were well tolerated with no observable toxicity.

### Example 8

Immunogenicity: The subcutaneous administration of Ab microparticle suspensions show no immune response presence:

Composition: The scope of the study involves four cohorts having the following compositions: 1) SC carrier liquid control: Ethyl Oleate; 10 μL, 2) SC mAb aqueous: 20 mg/mL Rituximab biosimilar (anti CD-20 antibody), 4 mg/mL Histidine, 1 mg/mL Trehalose, 0.7 mg/mL Sodium Chloride and 0.6 mg/mL Polysorbate 80; 10 μL, 3) SC mAb microparticle suspension: 20 mg/mL Rituximab biosimilar (anti CD-20 antibody), 4 mg/mL Histidine, 1 mg/mL Trehalose, 0.7 mg/mL Sodium Chloride and 0.6 mg/mL Polysorbate 80 formulated into particles which were then suspended in Ethyl Oleate (non-Aqueous carrier vehicle) with flocculation agent (PS80) at a concentration of 10 mg/mL; 10 μL, and 4) SC degraded aqueous mAb: Rituximab biosimilar (anti CD-20 antibody), subjected to heat and agitation to generate a positive control sample; 10 μL.

Animals: Female wild-type Balb/c mice (Charles River Labs) were housed in the animal facility at Tufts University Comparative Medicine Services (Tufts CMS). All animal procedures were formally reviewed and approved by the Institutional Animal Care and Use Committee (IACUC) at the Tufts CMS.

Dosing Study Design and Analysis: All particle composition injections were made using a 100 μL Hamilton glass syringe with a 27-gauge needle. All aqueous and non-aqueous vehicle injections were made using a 300 μL insulin syringe with staked-in 30-gauge needle. No protocol was implemented to resuspend the particles in the syringe. Balb/c female mouse at 10 weeks (Charles River Lab) was used for this study. The following study design was used: 1) SC carrier liquid control: Volume=10 μL; n=10, 2) SC Rituximab aqueous: Dose=10 mg/kg; Volume=10 μL; n=10, 3) SC Rituximab microparticle suspension: Dose=10 mg/kg; Volume=10 μL; n=10, 4) SC degraded Rituximab aqueous: Dose=10 mg/kg, Volume=10 μL; n=10. Plasma samples were collected at 0 d, and 35 d. The blood from each mouse was collected in EDTA capillary tubes from facial vein bleeds at all data points. The plasma obtained from the blood was diluted to 2,500× for analysis using Meso Scale Discovery® Electrochemiluminescence (MSD-ECL) Platform. The master mix was made with equal concentration (0.5 μg/ml) of SULFO-TAG and biotinylated Rituximab diluted in assay diluent (1% MSD Blocker A in PBS with 0.02% Tween20). An ADA standard curve was made by serial diluting mouse Anti-Rituximab Antibody (GenScript A01970-40) at 1000 ng/mL, 333 ng/mL, 111 ng/mL, 37 ng/mL, 12 ng/mL 4 ng/mL, 1 ng/mL and 0 ng/mL. First, 50 μL of the master mix was added to each well of a round-bottom 96-well polypropylene plate. Then, 25 μL of the ADA standard or 4% plasma sample was added to each well. The plate was sealed and incubated with moderate shaking overnight at 4° C. 150 μL of Blocking Solution (3% w/v MSD Blocker A in PBST) was added to each well of a 96-well Streptavidin plate (MSD L55SA). The plate was sealed and incubated with moderate shaking for at least 30 minutes at room temperature before washing with 200 μL PBST. A 50 μL solution was transferred from each well of the polypropylene plate to the Streptavidin plate. The plate was then sealed and incubated for 1 hour at room temperature with 400-700 rpm shaking, followed by 3 washes of 200 μL PBST. 150 μL 2× Read Buffer was added to each well. The plate was analyzed using an MSD QUICKPLEX instrument. The results of this study show that the subcutaneous administration of Ab microparticle suspensions show no immune response presence based on repeat-dose anti-drug antibody (immunogenicity) for rituximab.

### Example 9

Administration of hyaluronidase allows increased volume and fluid dispersion of the composition with flocculation agent administered by subcutaneous syringe injection:

Procedures similar to those described in Examples 1-4 were employed for particle and suspension production. Particle formulation: HIgG 74% protein: 2% histidine: 24% arginine: 1% PS80 provided smooth and spherical particles with a particle water content of 3%. FIB-SEM show particles have no internal void space. Suspension formulation: carrier liquid ethyl oleate, protein content 100 mg/mL, 1% PS80. The flocculation volume remained stable for at least 1 month at 25° C. SVPs >2 μm, measured by Aura Normalized per mg-protein (1.8E5), SEC 98.9% monomer data show the particles remained stable for at least 1 month at about 25° C.

Composition: The scope of the study involves one pork belly sample 8″×18″ double bagged (Savenor's, Boston) and equilibrated to physiological temperature having two sets of injection sites with the following compositions: Group 1 injection site for PBS solution; and Group 2 injection site for SC Ab suspension administered after a hyaluronidase pre-injection: i) 2000 U of hyaluronidase (Creative Biomart) in 8.5 mg/ml NaCl, 10 mM citric acid-sodium citrate, 0.9 mg/ml EDTA-2Na, 0.3 mg/ml CaCl₂) pH 5.2; followed by ii) SC Ab suspension. The concentration of the suspension was 100 mg/mL of protein. No protocol was implemented to resuspend the particles in the syringe.

Dosing. Study Design and Analysis: A five-pound portion of pork belly with skin was allowed to reach physiological temperature. The skin side was thoroughly cleansed using alcohol and then dried using a sterile gauze. No protocol was implemented to resuspend the particles in the syringe. While the skin was stretched taut, the PBS solution (Group 1) was subcutaneously injected under the skin at a 45-degree angle using a 24 mL Henke-Ject syringe and a 27G UTW needle. The suspension was dispensed until sufficient back pressure arose, preventing further dispensation of suspension. The volume dispensed was 1.4 mL and the average injection rate was 0.025 mL/sec. The experiment was repeated with SC Ab suspension subcutaneously administered after a hyaluronidase pre-injection (Group 2), 1 mL of the hyaluronidase mixture was subcutaneously pre-injected under the skin at a 45-degree angle using a 1 mL Tuberculin syringe and a 25G UTW needle. After a period of about 5-10 min, a 24 mL Henke-Ject syringe and 25G UTW needle was used to subcutaneously inject the SC Ab suspension into the same injection site at a 45-degree angle. The suspension was dispensed until sufficient back pressure prevented any further dispensing of the suspension. The average volume was 5.3 mL and the average injection rate was 0.045 mL/sec. The endpoints of the study included injection volume measurements and qualitative assessment of the injection site. All injections were conducted using the same injection force.

Results: The objective of this study is to determine the maximum feasible volume that can be dosed when given subcutaneously as a single dose. The experiments indicate that the injection volume of the SC Ab suspension that were subcutaneously administered (1.4 mL total volume) can be greatly increased by pre-injection with hyaluronidase (5.3 mL total volume) and these hyaluronidase injected sites showed improved rates of injection of the Ab particle composition as determined by the appearance of the skin bleb formed at the site of treatment and an decrease in the injection force (increase in injection rate) and a greater volume of the injection.

### Example 10

Procedures similar to Examples 1-4 were employed for particle and suspension production. Particle formulation: rituximab 160 mg/mL (98%), histidine 2.63 mg/mL (3%), pH: 6.0. Suspension formulation: carrier liquid ethyl oleate, protein content 620 mg/mL, solids load 843 mg/mL.

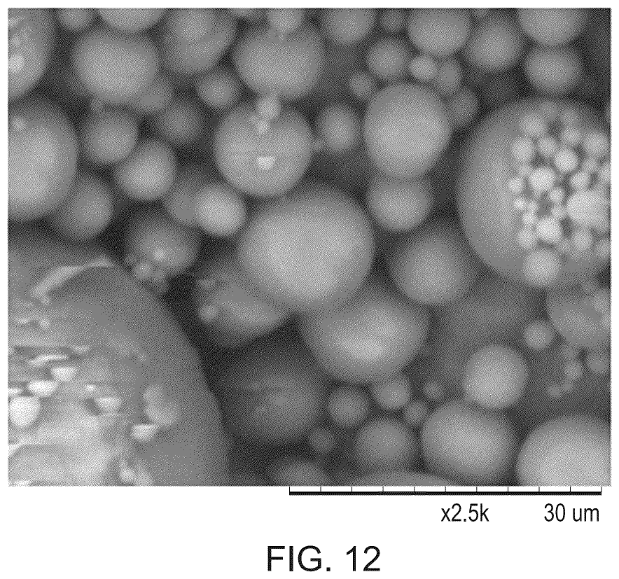

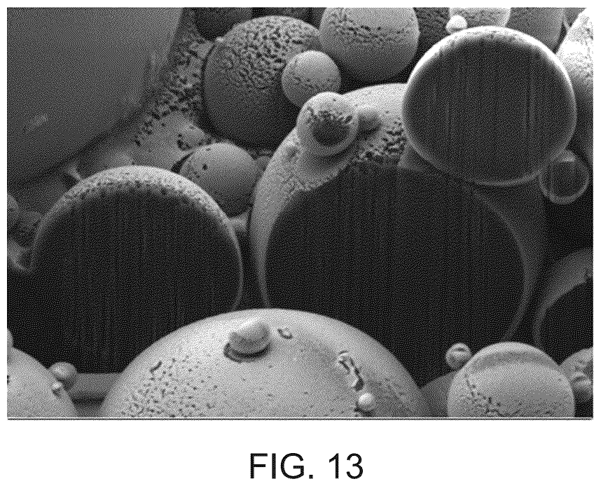

Particle production and droplet generation (Kinematica/20 mm) studies: diffuse phase: continuous phase (nBA) ratio 1/200, at least 16 mL:3.2 L, with a mixer speed of (10000 rpm)/60 s of mixing. Solid-liquid separation process by centrifugation and subsequent drying (air 10-75 RH) provided smooth and spherical particles with a particle size distribution (D10/D50/D90) of 9.34/20.32/40.58 μm with a particle water content of 3.4% (FIG. 12). FIB-SEM show particles have no internal void space (FIG. 13).

Fill volume of 2.56 mL using ethyl oleate as the carrier liquid. Suspension dispersion process and parameters using RSM 5000 rpm for 120 s and filter (60 um). Add 10 uL of a stock 100 mg/mL PS80 solution in ethyl oleate (1% PS80) to the suspension and vortex to mix. The flocculation volume remained stable for at least 3 months at 25° C. Transfer 1 mL of suspension into a 1 mL Gerresheimer 13 mm syringe and add a 25 gauge UTW needle to syringe. Measure syringe force at 0.05 mL/sec (51.1 mm/min) or at 0.1 mL/sec (189.4 mm/min) and 0.05 mL/sec (94.6 mm/min).

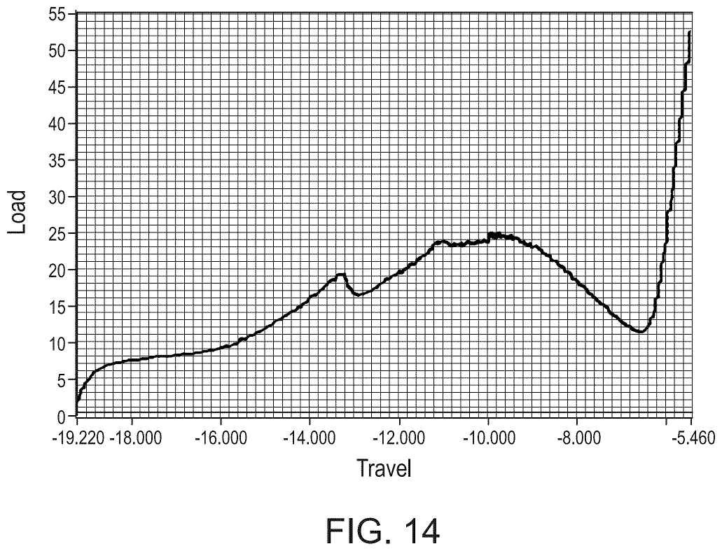

Syringe force results. FIG. 14 shows a glide force of a 620 mg/mL rituximab suspension when injected at 0.05 mL/sec to be 16 N.

Autoinjector force results. A syringe was loaded with the 620 mg/mL suspension and placed in a testing apparatus to simulate spring-driven autoinjectors. The spring was able to eject suspension from the syringe at 16 N.

### Example 11

Procedures similar to Examples 1-4 were employed for particle and suspension production. Particle formulation: rituximab 70 mg/mL (80%), histidine 2.2 mg/mL (2%), arginine·HCl 15.3 mg/mL (18%), pH: 6.0. Suspension formulation: carrier liquid ethyl oleate, protein content 507 mg/mL, solids load 735 mg/mL.

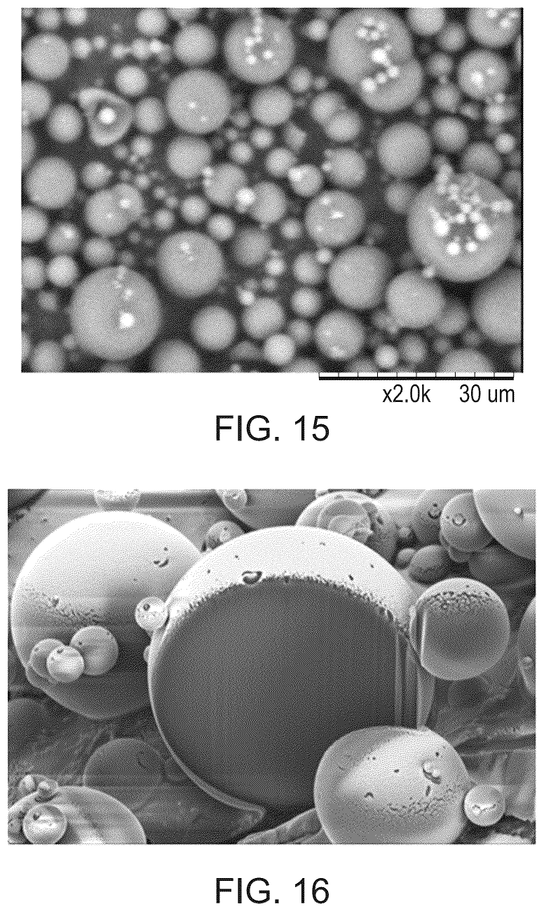

Particle production and droplet generation (Kinematica/20 mm) studies: diffuse phase: continuous phase (nBA) ratio 1/200, at least 20 mL:4 L, with a mixer speed of (8000 rpm)/60 s of mixing. Solid-liquid separation process by centrifugation and subsequent drying (air 10-75 RH) provided smooth and spherical particles with a particle size distribution (D10/D50/D90) of 6.6/11.4/19.3 μm with a particle water content of 3% (FIG. 15). FIB-SEM show particles have no internal void space (FIG. 16). SVPs >2 μm, measured by Aura Normalized per mg-protein (3.5E5), Pro-A, 16.00% Oxidized, SCEX (% Acidic/Neutral/Basic) 7.7/82.7/9.6, SEC 96.6% monomer data show the particles remained stable for at least 3 months.

Fill volume of 3.2 mL using ethyl oleate as the carrier liquid. Suspension dispersion process and parameters using RSM 6000 rpm for 15 min and filter (60 um). Add 10 uL of a stock 100 mg/mL PS80 solution in ethyl oleate (1% PS80) to the suspension and vortex to mix. The flocculation volume remained stable for at least 3 months at 25° C. Transfer 1 mL of suspension into a 2.25 mL Neopak syringe and add a 27 gauge STW×½″ needle to syringe. Measure syringe force at 0.1 mL/sec (189.4 mm/min).

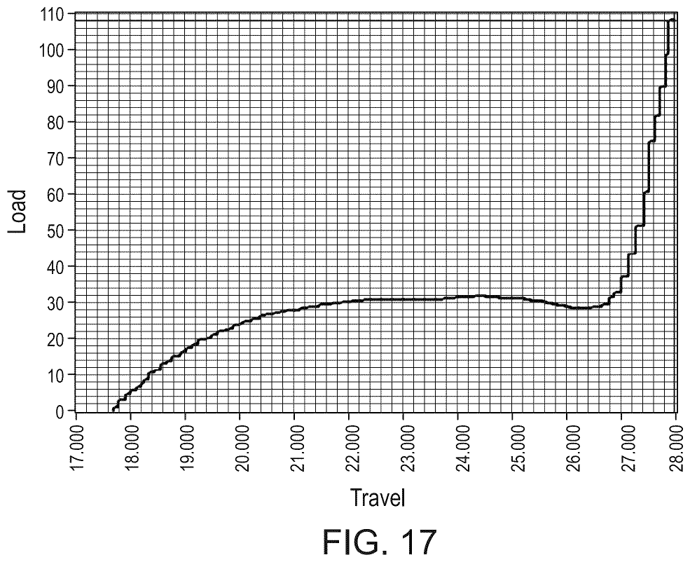

Syringe force results. FIG. 17 shows a glide force of a 507 mg/mL rituximab suspension when injected at 0.1 mL/sec to be 30 N.

### Example 12

Procedures similar to Examples 10 and 11 were employed for trastuzumab particle and suspension production. Particle formulation: trastuzumab 71.4 mg/mL (71%), histidine 2.2 mg/mL (2%), arginine·HCl 15.3 mg/mL (26%), PS80 0.9 mg/mL (1%), pH: 6.0. Suspension formulation: carrier liquid ethyl oleate, protein content 507 mg/mL, solids load 735 mg/mL.

Particle production and droplet generation (Kinematica/20 mm) studies: diffuse phase: continuous phase (nBA) ratio 1/200, at least 18 mL:3.6 L, with a mixer speed of (4000 rpm)/60 s of mixing. Solid-liquid separation process by centrifugation and subsequent drying (air 10-75 RH) provided smooth and spherical particles SEC monomer data show the particles remained stable for at least 3 months.

Flocculation volume and storage results. After 1 month, the flocculation volume remained stable at 100% with no observable sedimentation of the protein particles.

Syringe force results. After 1 month, the results showed no change in glide force of the 507 mg/mL trastuzumab suspension.

### Example 13

Procedures similar to Examples 1-4 were employed for particle and suspension production. Particle formulation: HIgG 74% protein: 2% histidine: 24% arginine: 1% PS80 provided smooth and spherical particles with a particle water content of 3%. FIB-SEM show particles have no internal void space. Suspension formulation: carrier liquid ethyl oleate, protein content 500 mg/mL, 1% PS80. The flocculation volume remained stable for at least 1 month at 25° C. SVPs >2 μm, measured by Aura Normalized per mg-protein (1.8E5), SEC 98.9% monomer data show the particles remained stable for at least 1 month at about 25° C.

Flocculation volume and storage stability results. After 1 month at 4° C., 25° C. and 40° C., the flocculation volume remained stable at 100% with no observable sedimentation of the protein particles. Storage stability of a 500 mg/mL HIgG suspension was tracked at 4° C., 25° C. and 40° C. with data collected at 1, 2 and 3 months with concentrations of PS80 in ethyl oleate at 0 mg/mL, 0.1 mg/mL and 1.0 mg/mL. The data showed that SVP>2 μm counts decreased with increasing concentration of PS80 formulated in ethyl oleate after storage at 4° C., 25° C. and 40° C. for up to 3 months. SVP counts decreased by at least 4× with 1.0 mg/mL of PS80 in EO for 500 mg/mL HIgG suspensions stored at 25° C. and 40° C. for at least 3 months.

Syringe force results. Transfer 1 mL of suspension into a 1 mL Schott SyriQ BioPure syringe and add a 27 gauge ½″ needle to syringe. Measure syringe force at 0.1 mL/sec (189.4 mm/min). Syringe force results show a glide force of a 500 mg/mL HIgG suspension when injected at 0.1 mL/sec to be 16 N.

Measurements for yield stress was acquired by a parallel plate rheometer (ANTON PAAR™ MCR 92). The results show that a yield stress was observed which indicates the formation of a stable flocculation volume allowing the particle suspension to flow upon the addition of a flocculation agent.

### Example 14

Particle compositions with more than one protein:

Procedures similar to Examples 10-12 were employed for the rituximab and trastuzumab suspension production. Particle formulation (Examples 11 and 12): rituximab 70 mg/mL (80%), histidine 2.2 mg/mL (2%), arginine·HCl 15.3 mg/mL (18%), pH: 6.0; and trastuzumab 71.4 mg/mL (71%), histidine 2.2 mg/mL (2%), arginine·HCl 15.3 mg/mL (26%), PS80 0.9 mg/mL (1%), pH: 6.0. Suspension formulation: carrier liquid ethyl oleate (2% PS80) to the suspension and vortex to mix, 221.2 mg of rituximab and 310.5 mg of trastuzumab for a total of 531.7 mg (protein content 400 mg/mL), particle protein percentage 70%.

Flocculation volume and suspension stability results. After 1 month, the flocculation volume remained stable at 100% with no observable sedimentation of the rituximab and trastuzumab particles. After 1 month, analysis of the particles and particle suspensions confirmed that protein quality slightly decreased as measured by the monomer profile by SEC when PS80 was formulated in ethyl oleate after storage at 25° C. for at least 1 month.

### Example 15

Protein particle compositions with at least two carrier liquids:

Procedures similar to Examples 1-4 were employed for particle and suspension production. Particle formulation: HIgG 130 mg/mL (88%), histidine 5.0 mg/mL (3%), arginine·HCl 9.8 mg/mL (7%), methionine 2.0 (1%), PS80 0.1 (1%), pH: 5.5. Suspension formulation: carrier liquid 40/60 ethyl oleate:sesame oil, protein content 400 mg/mL, solids load 452 mg/mL, flocculation agent: 0 or 2 mg/mL.

Particle production and droplet generation (Kinematica/20 mm) studies: diffuse phase: continuous phase (nBA) ratio 1/200, at least 20 mL:4 L, with a mixer speed of (8000 rpm)/60 s of mixing. Solid-liquid separation process by centrifugation and subsequent drying (air 10-75 RH) provided smooth and spherical particles with a particle size distribution (D10/D50/D90) of 6.1/10.1/15.7 μm with a particle water content of 3%. FIB-SEM show particles have no internal void space. SVPs >2 μm, measured by Aura Normalized per mg-protein (3.5E5), Pro-A, 16.00% Oxidized, SCEX (% Acidic/Neutral/Basic) 7.7/82.7/9.6, SEC 96.6% monomer data show the particles remained stable for at least 3 months.

Fill volume of 3.2 mL using 40/60 ethyl oleate:sesame oil as the carrier liquid. Suspension dispersion process and parameters using RSM 6000 rpm for 15 min and filter (60 um). Add 10 uL of a stock 100 mg/mL PS80 solution in ethyl oleate (1% PS80) to the suspension and vortex to mix. The flocculation volume remained stable for at least 1 months at 25° C. (74.8%) then compared to suspension without flocculation agent (62.3% after 1 month). Transfer 1 mL of suspension into a 2.25 mL Gerresheimer syringe and add a 27 gauge TW×½″ needle to syringe. Measure syringe force at 0.1 mL/sec (189.4 mm/min). Storage at room temperature, tip-up until no change in flocculation volume was observed (at least 1 month storage).

Flocculation volume and storage stability results. After 1 month at 25° C., the flocculation volume remained stable at 74.8% with no observable sedimentation of the protein particles, as compared to a suspension without flocculation agent (62.3% after 1 month). Storage stability of a 400 mg/mL HIgG suspension was tracked at 25° C. with data collected at 1 month with concentrations of PS80 in 40/60 ethyl oleate:sesame oil at 0 mg/mL and 0.1 mg/mL. The data showed that SVP>2 μm counts decreased with increasing concentration of PS80 formulated in 40/60 ethyl oleate:sesame oil after storage at 25° C. for up to at least 1 month.

Syringe force results. Transfer 1 mL of suspension into a 1 mL Gerresheimer syringe and add a 27 gauge TW× ½″ needle to syringe. Measure syringe force at 0.1 mL/sec (189.4 mm/min). Syringe force results show a glide force of a 400 mg/mL HIgG suspension with flocculation agent using 40/60 ethyl oleate:sesame oil as the carrier liquid when injected at 0.1 mL/sec to be 20.1 N, when compared to a suspension without flocculation agent was found to be 27.4 N. Viscosity was found to be 23.3 cP, compared to a suspension without flocculation agent (31.8 cP).

Measurements for yield stress was acquired by a parallel plate rheometer (ANTON PAAR™ MCR 92). The results show that a yield stress was observed which indicates the formation of a stable flocculation volume allowing the particle suspension to flow upon the addition of a flocculation agent.

### Example 16

Protein particle compositions with MCT as the carrier liquid:

Procedures similar to Examples 1-4 were employed for particle and suspension production. Particle formulation: HIgG 130 mg/mL (88%), histidine 5.0 mg/mL (3%), arginine·HCl 9.8 mg/mL (7%), methionine 2.0 (1%), PS80 0.1 (1%), pH: 5.5. Suspension formulation: carrier liquid MCT, protein content 400 mg/mL, solids load 452 mg/mL, flocculation agent: 0 or 2 mg/mL.

Particle production and droplet generation (Kinematica/20 mm) studies: diffuse phase: continuous phase (nBA) ratio 1/200, at least 20 mL:4 L, with a mixer speed of (8000 rpm)/60 s of mixing. Solid-liquid separation process by centrifugation and subsequent drying (air 10-75 RH) provided smooth and spherical particles with a particle size distribution (D10/D50/D90) of 6.1/10.1/15.7 μm with a particle water content of 3%. FIB-SEM show particles have no internal void space. SVPs >2 μm, measured by Aura Normalized per mg-protein (3.5E5), Pro-A, 16.00% Oxidized, SCEX (% Acidic/Neutral/Basic) 7.7/82.7/9.6, SEC 96.6% monomer data show the particles remained stable for at least 3 months.

Fill volume of 3.2 mL using MCT as the carrier liquid. Suspension dispersion process and parameters using RSM 6000 rpm for 15 min and filter (60 um). Add 10 uL of a stock 100 mg/mL PS80 solution in MCT (1% PS80) to the suspension and vortex to mix. The flocculation volume remained stable for at least 1 month at 25° C. (87.6%) then compared to suspension without flocculation agent (65.1% after 1 month). Transfer 1 mL of suspension into a 2.25 mL Gerresheimer syringe and add a 27 gauge TW×½″ needle to syringe. Measure syringe force at 0.1 mL/sec (189.4 mm/min). Storage at room temperature, tip-up until no change in flocculation volume was observed (at least 1 month storage).

Flocculation volume and storage stability results. After 1 month at 25° C., the flocculation volume remained stable at 87.6% with no observable sedimentation of the protein particles, as compared to a suspension without flocculation agent (65.1% after 1 month). Storage stability of a 400 mg/mL HIgG suspension was tracked at 25° C. with data collected at 1 month with concentrations of PS80 in MCT at 0 mg/mL and 0.1 mg/mL. The data showed that SVP>2 μm counts decreased with increasing concentration of PS80 formulated in MCT after storage at 25° C. for up to at least 1 month.

Syringe force results. Transfer 1 mL of suspension into a 1 mL Gerresheimer syringe and add a 27 gauge TW×1/2″ needle to syringe. Measure syringe force at 0.1 mL/sec (189.4 mm/min). Syringe force results show a glide force of a 400 mg/mL HIgG suspension with flocculation agent using 40 MCT as the carrier liquid when injected at 0.1 mL/sec to be 31.8 N, when compared to a suspension without flocculation agent was found to be 38.9 N. Viscosity was found to be 36.9 cP, compared to a suspension without flocculation agent (45.2 cP).

Measurements for yield stress was acquired by a parallel plate rheometer (ANTON PAAR™ MCR 92). The results show that a yield stress was observed which indicates the formation of a stable flocculation volume allowing the particle suspension to flow upon the addition of a flocculation agent.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific aspects and embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

What is claimed is:

1. A pharmaceutically effective composition, comprising a suspension of a plurality of particles comprising a therapeutic biologic or salt thereof in a suspension medium, wherein:
the suspension medium comprises a pharmaceutically acceptable liquid carrier and a flocculation agent;
the concentration of the therapeutic biologic or salt thereof in the composition is greater than about 250 mg/mL.

2. The composition of claim 1, wherein the concentration of the flocculation agent in the composition is from 0.1 mg/mL to 10 mg/mL.

3. The composition of claim 1, wherein the flocculation agent is ionic.

4. The composition of claim 3, wherein the ionic flocculation agent is magnesium stearate, sodium dodecyl sulfate, sodium stearate, cetyltrimethylammonium bromide, lecithin, or a combination thereof.

5. The composition of claim 1, wherein the flocculation agent is non-ionic.

6. The composition of claim 5, wherein the non-ionic flocculation agent is a polysorbate, an alkylphenol ethoxylate, glycerol, polyoxyethylated castor oil, docusate, decyl glucoside, nonoxynol-9, a sorbitan ester, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof.

7. The composition of claim 1, wherein the composition after 30 days of storage at about 40° C. has less than about 1° C. thermal shift in its differential scanning fluorimetry (DSF) profile compared to its DSF profile prior to storage.

8. The composition of claim 1, wherein the composition increases in flocculation volume after initial mixing.

9. The composition of claim 8, wherein the composition has a flocculation volume greater than about 60% after the initial mixing.

10. The composition of claim 8, wherein the flocculation volume is reduced by less than about 10% after at least one month under container closure storage conditions at less than about 40° C.

11. The composition of claim 1, wherein the composition has substantially the same flocculation volume for at least one month under container closure storage conditions at less than about 40° C.

12. The composition of claim 1, wherein the plurality of particles and flocculation agent remain substantially suspended in the liquid carrier for at least one month under container closure storage conditions at less than about 40° C.

13. The composition of claim 1, wherein substantially all of the particles have less than about 10% aggregation of the therapeutic biologic.

14. The composition of claim 1, wherein substantially all of the particles have less than about 10% fragmentation of the therapeutic biologic.

15. The composition of claim 1, wherein substantially all of the particles have less than about 5% change in charge variants of the therapeutic biologic.

16. The composition of claim 1, wherein substantially all of the particles have a circularity of about 0.80 to about 1.00.

17. The composition of claim 1, wherein substantially all of the particles have less than about 7% moisture by weight.

18. The composition of claim 1, wherein substantially all of the particles have less than about 10% internal void spaces.

19. The composition of claim 1, wherein substantially all of the particles have greater than about 60% therapeutic biologic by weight.

20. The composition of claim 1, wherein substantially all of the particles further comprise a carbohydrate, a pH adjusting agent, a salt, a chelator, a surfactant, a protein stabilizer, an emulsifier, an antiseptic, an amino acid, an antioxidant, a paraben, a bactericide, a fungicide, a preservative, or a combination thereof.

21. The composition of claim 20, wherein the pH adjusting agent is acetate, citrate, glutamate, glycinate, histidine, lactate, maleate, phosphate, succinate, tartrate, bicarbonate, aluminum hydroxide, phosphoric acid, hydrochloric acid, DL-lactic/glycolic acids, phosphorylethanolamine, tromethamine, imidazole, glyclyglycine, monosodium glutamate, sodium hydroxide, potassium hydroxide, or a combination thereof.

22. The composition of claim 20, wherein the chelator is disodium edetate, ethylenediaminetetraacetic acid pentetic acid, or a combination thereof.

23. The composition of claim 1, wherein the liquid carrier comprises ethyl oleate, ethyl caprylate, propylene glycol dicaprylate, diglycerides, trigylcerides, sesame oil, propylene glycol diesters of saturated plant fatty acids C8 and C10 (PGD), triacetin, or a combination thereof.

24. The composition of claim 1, wherein the therapeutic biologic is an antibody or fragment thereof.

25. The composition of claim 1, wherein the concentration of the therapeutic biologic or salt thereof in the composition is greater than about 400 mg/mL.

26. The composition of claim 1, wherein the concentration of the flocculation agent in the composition is from 0.01% to 0.1% by weight.
